(19) United States
(12) Patent Application Publication
(10) Pub. No.: US 2025/0382317 A1
(43) Pub. Date: Dec. 18, 2025

(54) N-SUBSTITUTED INDOLE DERIVATIVES AND CONJUGATES FOR THE TREATMENT OF CANCER

(71) Applicant: EVEXTA BIO, PARIS (FR)

(72) Inventors: CÉCILE BOUGERET, PARIS (FR); DOMINIQUE BRIDON, PARIS (FR); DENIS CARNIATO, PARIS (FR)

(21) Appl. No.: 18/878,688

(22) PCT Filed: Jun. 27, 2023
(86) PCT No.: PCT/EP2023/067379

§ 371 Date: Dec. 24, 2024

(30) Foreign Application Priority Data
Jun. 27, 2022 (EP) 22305927.0

(51) Int. Cl. C07F 9/572; A61K 47/64; A61K 47/68; C07H 15/26

(52) U.S. Cl. CPC C07F 9/5728; A61K 47/643; A61K 47/6803; C07H 15/26

(57) ABSTRACT

The present invention relates to N-substituted derivatives of indoles of formula (I):

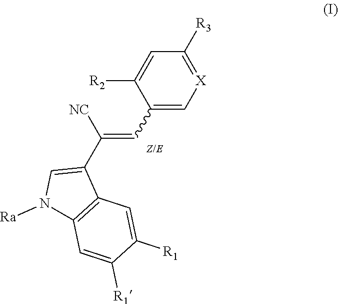

and their use in the treatment of cancer. The invention further provides protein-drug conjugates, more particularly antibody-drug conjugates, from compounds of formula (I).

### FIELD OF THE INVENTION

The present invention relates to the medicinal field and, more specifically, to N-substituted derivatives of indoles and their uses as therapeutics compounds. Particularly, such derivatives are used in the treatment of cancer. Some of them are also used in the manufacture of conjugates with a protein or an antibody.

### BACKGROUND OF THE INVENTION

Kinesins are a superfamily of motor proteins that have ATP enzyme activity. They are involved in the normal biological activities of various cells, including mitosis, meiosis, and intracellular vesicle transport. Kinesin family member 20A (KIF20A, also known as MKlp2) is located on chromosome 5q31.2 and plays an important role in the occurrence and development of tumors. Recently, several studies have demonstrated that KIF20A may play an important role in the development and progression of many different types of cancer, such as melanoma, breast cancer, nasopharyngeal cancer, pancreatic cancer, hepatocellular carcinoma, lung cancer, and colorectal cancer.

In past years, a class of indole derivatives as compounds targeting MKlp2 (KIF20A) has been developed such as compounds described in the international patent application WO 2014/086964. These compounds correspond to selective inhibitors for MKlp2 (KIF20A) at a nanomolar efficiency despite they have a poor solubility in water at both neutral and acid pH.

It is well known that solubility is one of the most important parameters to achieve desired concentration of drugs in systemic circulation for a pharmacological response whatever the administration used. Indeed, low aqueous solubility is a key problem encountered with the development of pharmacological chemical entities and formulation comprising them.

Accordingly, the water solubility of this class of indole derivatives needs to be enhanced to guarantee the solubility of such derivatives at the site of absorption.

### SUMMARY OF THE INVENTION

In this context, the inventors have provided highly soluble prodrugs in water derived from indole derivatives, particularly indoles derivatives reported in WO 2014/086964. More particularly, the nitrogen atom of the indole has been linked either to phosphonic acid containing promoieties or to a cleavable group, for instance a metabolic liable group. The cleavable group can also comprise a binding protein connector to provide protein conjugates, for instance with albumin, for increasing circulating half-life of the drug and promote intracellular and/or extracellularly release of these drugs to tumors, for instance, by increasing internalization of these drugs within cancer cells by antibody conjugates.

The present invention thus relates to a compound of formula (I):

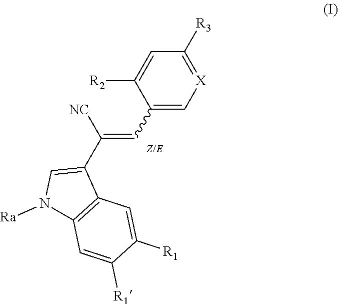

wherein:
X represents a nitrogen atom, a C—CN unit or a N+—O— unit;
R₁ and R₁′ represent independently a hydrogen, a halogen, a (C₁-C₆)alkoxy, or a —SO₂—CH₃ group, with the proviso that if R₁ or R₁′ is a (C₁-C₆)alkoxy, then R² is not a halogen;
—R² represents:
   a radical (C₁-C₆)alkoxy, (C₃-C₆)cycloalkoxy, (C₃-C₆) heterocycloalkoxy, aryloxy, heteroaryloxy, (C₁-C₆)alkyl-aryloxy, (C₁-C₆)alkyl-heteroaryloxy, said radicals being optionally substituted by at least one halogen, or a radical thio-(C₁-C₆)alkyl, thio-aryl, thio-heteroaryl, thio-(C₁-C₆)alkyl-aryl or thio-(C1-C₆)-alkyl-heteroaryl, said radicals being optionally substituted by at least one halogen or by a (C₁-C₆)alkoxy group,
   a —NR₄R₅ unit, a O—(C₁-C₆)alkyl-NR₄R₅ unit or a S—(C₁-C₆)alkyl-NR₄R₅ unit wherein R⁴ and R₅ represent H, a (C₁-C₆)alkyl group, or R₄ and R⁵ taken together form a 3- to 7-membered ring, optionally interrupted by one or several heteroatoms, with the proviso that at least one among R₄ and R⁵ is not H,
   “a NHCOR₆ unit wherein R⁶ represents (C₁-C₆)alkyl group,”
   an aryl or a heteroaryl group optionally substituted by at least one halogen, a trifluoromethyl group, or a (C₁-C₃)alkoxy group, or
   a halogen,
R₃ represents a hydrogen, a (C₁-C₃)alkyl group, a (C₁-C₃)alkoxy group or a halogen; and
Ra is a radical selected in a group consisting of:
   a group of formula (A):

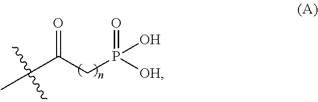

or formula (A′):

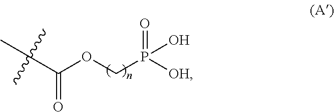

in which n is an integer comprised between 1 and 12; and
a group of formula (B):

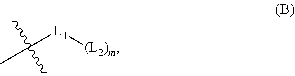

in which:
   L₁ is a cleavable group chosen among a pH-sensitive group, a photo-induced cleavable group, a bioreductible cleavable group, and an enzymatic cleavable group;
   L₂ is a tert-butoxycarbonyl group or a binding protein connector, and
   m is an integer equal to 0 or 1;
or a pharmaceutically acceptable salt thereof.

In a particular embodiment, R₂ represents:
a radical (C₁-C₆)alkoxy, preferably a methoxy group,
a radical (C₃-C₆) heterocycloalkoxy, preferably an oxetanoxy group, or
a heteroaryl group, preferably a furanyl or a triazolyl group.

In a preferred embodiment, the compound of formula (I) is such that:
X represents a C—CN unit,
R₁ represents a halogen, preferably a bromine,
R₁′ represents a hydrogen,
R₂ represents a radical (C₁-C₆)alkoxy, preferably a methoxy group, and
R₃ represents a hydrogen.

In a further preferred embodiment, the compound of formula (I) is such that:
X represents a C—CN unit,
R₁ represents a hydrogen,
R₁′ represents a radical (C₁-C₆)alkoxy, preferably a methoxy group,
R₂ represents a radical (C₁-C₆)alkoxy, preferably a methoxy group, and
R₃ represents hydrogen.

In an embodiment of the invention, the compound of formula (I) or its pharmaceutically acceptable salt is such that Ra is a group of formula (A):

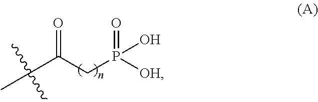

or formula (A′):

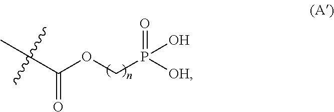

in which n is an integer comprised between 1 and 12.

Preferably, n is an integer comprised between 2 and 10. More preferably, n is an integer comprised between 2 and 8, advantageously between 2 and 6. Even more preferably, n is 2, 3, 4, 5, or 6.

In a particular embodiment, the pharmaceutically acceptable salt of the compound of formula (I) is chosen among a sodium salt, a disodium salt, a lysine salt, a dilysine salt, an arginine salt or a diarginine salt, preferably a sodium salt or a disodium salt, more preferably a sodium salt. In a preferred embodiment, the compound of formula (I) is selected from the group consisting of:
Example 1: (Z)-5-(5-bromo-3-(1-cyano-2-(5-cyano-2-methoxyphenyl)vinyl)-1H-indol-1-yl)-5-oxopentylphosphonic acid;
Example 2: (Z)-3-(5-bromo-3-(1-cyano-2-(5-cyano-2-methoxyphenyl)vinyl)-1H-indol-1-yl)-3-oxopropylphosphonic acid;
Example 3: (Z)-7-(5-bromo-3-(1-cyano-2-(5-cyano-2-methoxyphenyl)vinyl)-1H-indol-1-yl)-7-oxoheptylphosphonic acid;
Example 4: (Z)-5-(3-(1-cyano-2-(5-cyano-2-methoxyphenyl)vinyl)-6-methoxy-1H-indol-1-yl)-5-oxopentylphosphonic acid;
Example 5: (Z)-7-(3-(1-cyano-2-(5-cyano-2-methoxyphenyl)vinyl)-6-methoxy-1H-indol-1-yl)-7-oxoheptylphosphonic acid;
Example 6: (Z)-hydrogen5-(5-bromo-3-(1-cyano-2-(5-cyano-2-Sodium methoxyphenyl)vinyl)-1H-indol-1-yl)-5-oxopentylphosphonate;
Example 7: sodium (Z)-hydrogen3-(5-bromo-3-(1-cyano-2-(5-cyano-2-methoxyphenyl)vinyl)-1H-indol-1-yl)-3-oxopropylphosphonate;
Example 8: sodium (Z)-hydrogen4-(5-bromo-3-(1-cyano-2-(5-cyano-2-methoxyphenyl)vinyl)-1H-indol-1-yl)-4-oxobutylphosphonate;
Example 9: sodium (Z)-hydrogen6-(5-bromo-3-(1-cyano-2-(5-cyano-2-methoxyphenyl)vinyl)-1H-indol-1-yl)-4-oxohexylphosphonate;
Example 10: sodium (Z)-hydrogen7-(5-bromo-3-(1-cyano-2-(5-cyano-2-methoxyphenyl)vinyl)-1H-indol-1-yl)-7-oxoheptylphosphonate;
Example 11: sodium (Z)-hydrogen5-(3-(1-cyano-2-(5-cyano-2-methoxyphenyl)vinyl)-6-methoxy-1H-indol-1-yl)-5-oxopentylphosphonate;
Example 12: sodium (Z)-hydrogen7-(3-(1-cyano-2-(5-cyano-2-methoxyphenyl)vinyl)-6-methoxy-1H-indol-1-yl)-7-oxoheptylphosphonate;
Example 13: disodium (Z)-4-(5-bromo-3-(1-cyano-2-(5-cyano-2-methoxyphenyl)vinyl)-1H-indol-1-yl)-4-oxobutylphosphonate;
Example 14: disodium (Z)-5-(5-bromo-3-(1-cyano-2-(5-cyano-2-methoxyphenyl)vinyl)-1H-indol-1-yl)-5-oxopentylphosphonate;
Example 15: (S)-2,6-diaminohexanoic acid compound with (Z)-3-(5-bromo-3-(1-cyano-2-(5-cyano-2-methoxyphenyl)vinyl)-1H-indol-1-yl)-3-oxopropylphosphonic acid (2:1);
Example 16: (S)-2,6-diaminohexanoic acid compound with (Z)-4-(5-bromo-3-(1-cyano-2-(5-cyano-2-methoxyphenyl)vinyl)-1H-indol-1-yl)-4-oxobutylphosphonic acid (2:1);
Example 17: 3-[hydroxy(dioxo)-lambda6-phosphanyl]propyl 5-bromo-3-[(Z)-1-cyano-2-(2-cyano-5-methoxy-phenyl)vinyl]indole-1-carboxylate; and
Example 18: 4-[hydroxy(dioxo)-lambda6-phosphanyl]butyl 5-bromo-3-[(Z)-1-cyano-2-(2-cyano-5-methoxy-phenyl)vinyl]indole-1-carboxylate.

In another embodiment of the invention, the compound of formula (I) or its pharmaceutically acceptable salt is such that Ra is a group of formula (B):

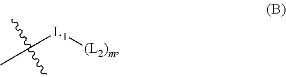

in which:
L₁ is a cleavable group chosen among a pH-sensitive group, a photo-induced cleavable group, a bioreductible cleavable group, and an enzymatic cleavable group;
L₂ is a tert-butoxycarbonyl group or a binding protein connector, and
m is an integer equal to 0 or 1.

In a particular embodiment, L₁ is an enzymatic cleavable group cleaved by a protease, a peptidase, an esterase, a beta-glucuronidase, a glycosidase, a phosphodiesterase, a phosphatase, a pyrophosphatase, a tubulin-tyrosine ligase or a lipase.

In a preferred embodiment, L₁ is a p-aminobenzyloxycarbonyl-AA1_{w}-AA2ₓ-AA3_{y}-AA4_{z} group with AA1, AA2, AA3, and AA4 represent independently an amino acid selected in a group consisting of alanine, valine, citrulline, phenylalanine, lysine, glycine, aspartic acid, asparagine, glutamic acid, and derivatives thereof, preferably citrulline, valine, cysteic acid, glycine, and glutamic acid, more preferably citrulline, valine, and cysteic acid, and w, x, y, and z are independently an integer equal to 0 or 1.

According to a particular embodiment, m is 1, and L₂ is a binding protein connector having the following formulae:

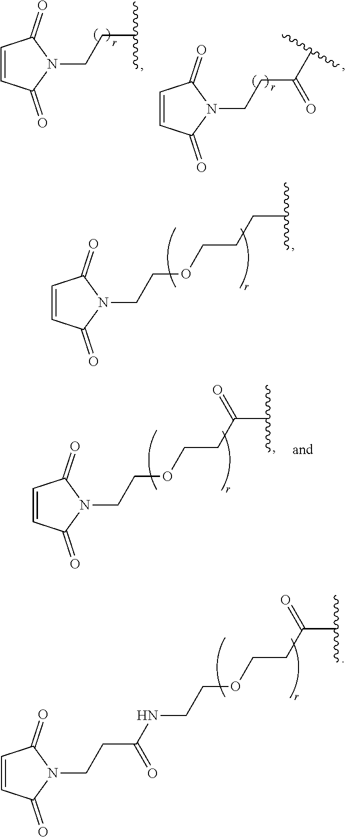

with r is an integer comprised between 1 and 36, preferably between 1 and 24, more preferably equal to 2, 4, 8, 12, 16, 20, or 24.

In a preferred embodiment, the compound of formula (I) is selected from the group consisting of:
Example 19: (2S,3S,4S,5R,6S)-6-[4-[[5-bromo-3-[(Z)-1-cyano-2-(5-cyano-2-methoxy-phenyl)vinyl]indole-1-carbonyl]oxymethyl]phenoxy]-3,4,5-trihydroxy-tetrahydropyran-2-carboxylic acid;
Example 20: [4-[[(2S)-2-[[(2S)-2-(tert-butoxycarbonylamino)-3-methyl-butanoyl]amino]-5-ureido-pentanoyl]amino]phenyl]methyl-5-bromo-3-[(Z)-1-cyano-2-(5-cyano-2-methoxy-phenyl)vinyl]indole-1-carboxylate;
Example 21: [4-[[(2S)-2-[[(2S)-2-aminopropanoyl]amino]-5-ureido-pentanoyl]amino]phenyl]methyl-5-bromo-3-[(Z)-1-cyano-2-(5-cyano-2-methoxy-phenyl)vinyl]indole-1-carboxylate hydrochloride;
Example 22: [4-[[(2S)-2-[[(2S)-2-[3-[2-[3-(2,5-dioxopyrrol-1-yl)propanoylamino]ethoxy](peg4) propanoylamino]-3-methyl-butanoyl]amino]-5-ureido-pentanoyl]amino]phenyl]methyl-5-bromo-3-[(Z)-1-cyano-2-(5-cyano-2-methoxy-phenyl)vinyl]indole-1-carboxylate;
Example 23: [4-[[(2S)-2-[[(2S)-2-[3-[2-[3-(2,5-dioxopyrrol-1-yl)propanoylamido](peg24)propanoylamino]-3-methyl-butanoyl]amino]-5-ureido-pentanoyl]amino]phenyl]methyl-5-bromo-3-[(Z)-1-cyano-2-(5-cyano-2-methoxy-phenyl)vinyl]indole-1-carboxylate;
Example 24: 3-[[(1S)-1-[(1S)-1-[[4-[[5-bromo-3-[(Z)-1-cyano-2-(5-cyano-2-methoxy-phenyl)vinyl]indole-1-carbonyl]oxymethyl]phenyl]carbamoyl]-4-ureido-butyl]carbamoyl]-2-methyl-propyl]amino]-2-(tert-butoxycarbonylamino)-3-oxo-propane-1-sulfonic acid;
Example 25: 2-amino-3-[[(1S)-1-[[(1S)-1-[[4-[[5-bromo-3-[(Z)-1-cyano-2-(5-cyano-2-methoxy-phenyl)vinyl]indole-1-carbonyl]oxymethyl]phenyl]carbamoyl]-4-ureido-butyl]carbamoyl]-2-methyl-propyl]amino]-3-oxo-propane-1-sulfonic acid; and
Example 26: (2R)-3-[[(1S)-1-[[(1S)-1-[[4-[[5-bromo-3-[(Z)-1-cyano-2-(5-cyano-2-methoxy-phenyl)vinyl]indole-1-carbonyl]oxymethyl]phenyl]carbamoyl]-4-ureido-butyl]carbamoyl]-2-methyl-propyl]amino]-2-[3-[2-[3-(2,5-dioxopyrrol-1-yl)propanoylamino]ethoxy]propanoylamino]-3-oxo-propane-1-sulfonic acid.

A further object of the invention is a pharmaceutical composition comprising a compound of the invention, and a pharmaceutically acceptable excipient.

Particularly, such a pharmaceutical composition is for use in the treatment of cancer. Preferably, cancer is chosen among leukemia, acute myelogenous leukemia, lymphoma, breast cancer, pancreatic cancer, lung cancer or colon cancer.

A further object of the invention is a conjugate of formula (II):

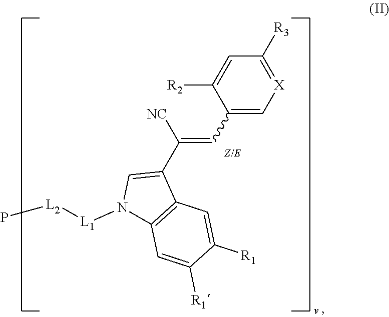

in which:
X, R₁, R₁, R₂, R₃, and L₁ are such as defined herein;
L₂ is a binding protein connector;
P is a peptide or a protein, preferably an antibody, an antibody fragment, or an antigen-binding fragment, which is able to bind a target of interest; and
v is an integer from 1 to 10.

### LEGEND OF FIGURES

FIG. 1: Cathepsin-B mediated drug release test

Drug concentration (μM) release of drug after incubation of quenched mal prodrug 20 μg/mL at 0-5-10-15-30-60 min.

### DETAILED DESCRIPTION OF THE INVENTION

### Definitions

According to the present invention, the terms below have the following meanings:

The terms mentioned herein with prefixes such as for example C₁-C₃, C₁-C₆ or C₂-C₆ can also be used with lower numbers of carbon atoms such as C₁-C₂, C₁-C₅, or C₂-C₅. If, for example, the term C₁-C₃ is used, it means that the corresponding hydrocarbon chain may comprise from 1 to 3 carbon atoms, especially 1, 2 or 3 carbon atoms. If, for example, the term C₁-C₆ is used, it means that the corresponding hydrocarbon chain may comprise from 1 to 6 carbon atoms, especially 1, 2, 3, 4, 5 or 6 carbon atoms. If, for example, the term C₂-C₆ is used, it means that the corresponding hydrocarbon chain may comprise from 2 to 6 carbon atoms, especially 2, 3, 4, 5 or 6 carbon atoms.

The term “alkyl” refers to a saturated, linear or branched aliphatic group. The term “(C₁-C₃)alkyl” more specifically means methyl, ethyl, propyl, or isopropyl. The term “(C₁-C₆)alkyl” more specifically means methyl, ethyl, propyl, isopropyl, butyl, isobutyl, tert-butyl, pentyl or hexyl. In a preferred embodiment, the “alkyl” is a methyl, an ethyl, a propyl, an isopropyl, or a tert-butyl, more preferably a methyl.

The term “alkoxy” or “alkyloxy” corresponds to the alkyl group as above defined bonded to the molecule by an —O— (ether) bond. (C₁-C₃)alkoxy includes methoxy, ethoxy, propyloxy, and isopropyloxy. (C₁-C₆)alkoxy includes methoxy, ethoxy, propyloxy, isopropyloxy, butyloxy, isobutyloxy, tert-butyloxy, pentyloxy and hexyloxy. In a preferred embodiment, the “alkoxy” or “alkyloxy” is a methoxy.

The term “thio” corresponds to the alkyl group defined hereinabove bounded to the molecule by a —S-(thioether) bound. Thio-(C₁-C₆)alkyl group includes thio-methyl, thio-ethyl, thio-propyl, thio-butyl, thio-pentyl and thio-hexyl.

The term “halogen” corresponds to a fluorine, chlorine, bromine, or iodine atom, preferably a fluorine, chlorine or bromine, and more preferably a chlorine or a bromine.

The term “aryl” corresponds to a mono- or bi-cyclic aromatic hydrocarbons having from 6 to 12 carbon atoms. For instance, the term “aryl” includes phenyl, biphenyl, or naphthyl. In a preferred embodiment, the aryl is a phenyl.

The term “heteroaryl” as used herein corresponds to an aromatic, mono- or poly-cyclic group comprising between 5 and 14 atoms and comprising at least one heteroatom such as nitrogen, oxygen or sulphur atom. Examples of such mono- and poly-cyclic heteroaryl group may be: pyridinyl, thiazolyl, thiophenyl, furanyl, pyrrolyl, pyrazolyl, imidazolyl, triazolyl, tetrazolyl, benzofuranyl, thianaphthalenyl, indolyl, indolinyl, quinolinyl, isoquinolinyl, benzimidazolyl, tetrahydroquinolinyl, tetrahydroisoquinolinyl, triazinyl, thianthrenyl, isobenzofuranyl, chromenyl, xanthenyl, phenoxanthinyl, isothiazolyl, isoxazolyl, pyrazinyl, pyridazinyl, indolizinyl, isoindolyl, indazolyl, purinyl, quinolizinyl, phtalazinyl, naphthyridinyl, quinoxalinyl, quinazolinyl, cinnolinyl, pteridinyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, indolinyl, isoindolinyl, oxazolidinyl, benzotriazolyl, benzoisoxazolyl, oxindolyl, benzoxazolinyl, benzothienyl, benzothiazolyl, isatinyl, dihydropyridyl, pyrimidinyl, s-triazinyl, oxazolyl, or thiofuranyl. In a preferred embodiment, the heteroaryl group is a pyridinyl, furanyl, pyrazolyl, pyrazinyl, pyrimidinyl, pyridazinyl, and isoxazolyl. Preferably, heteroaryl is pyridyl, thiazolyl, furanyl, pyranyl, pyrrolyl, imidazolyl, tetrazolyl, benzofuranyl, pyrrolinyl, triazinyl, pyrazinyl, pyridazinyl, triazolyl or tetrazolyl. More preferably, heteroaryl is furanyl or triazolyl.

The term “cycloalkyl” corresponds to a saturated or unsaturated mono-, bi- or tri-cyclic alkyl group comprising between 3 and 20 atoms of carbons. It also includes fused, bridged, or spiro-connected cycloalkyl groups. The term “cycloalkyl” includes for instance cyclopropyl, cyclobutyl, cyclopentyl, and cyclohexyl. In a preferred embodiment, the “cycloalkyl” is a cyclopropyl, cyclobutyl, cyclopentyl or a cyclohexyl.

The term “heterocycloalkyl” corresponds to a saturated or unsaturated cycloalkyl group as above defined further comprising at least one heteroatom such as nitrogen, oxygen, or sulphur atom. It also includes fused, bridged, or spiro-connected heterocycloalkyl groups. Representative heterocycloalkyl groups include, but are not limited to 3-dioxolane, benzo[1,3]dioxolyl, azetidinyl, oxctanyl, pyrazolinyl, pyranyl, thiomorpholinyl, pyrazolidinyl, piperidyl, piperazinyl, 1,4-dioxanyl, imidazolinyl, pyrrolinyl, pyrrolidinyl, piperidinyl, imidazolidinyl, morpholinyl, 1,4-dithianyl, pyrrolidinyl, oxozolinyl, oxazolidinyl, isoxazolinyl, isoxazolidinyl, thiazolinyl, thiazolidinyl, isothiazolinyl, isothiazolidinyl, dihydropyranyl, tetrahydropyranyl, tetrahydrofuranyl, and tetrahydrothiophenyl.

The terms “cycloalkoxy”, “heterocycloalkoxy”, “aryloxy”, and “heteroaryloxy”, correspond to the “cycloalkyl”, “heterocycloalkyl”, “aryl”, and “heteroaryl” as above defined bonded the molecule by an —O— (ether) bond. For instance, “cycloalkoxy”, “heterocycloalkoxy”, “aryloxy”, and “heteroaryloxy” correspond to —O-cycloalkyl, —O-heterocycloalkyl, —O-aryl, and —O-heteroaryl. A preferred embodiment of a “heterocycloalkoxy” is an oxetanyloxy or oxetanoxy (—O-oxctanyl).

The term “Boc” corresponds to the following group tert-butoxycarbonyl “—C(═O)—O—C(CH₃)₃”. The expression “substituted by at least” means that the radical is substituted by one or several groups of the list.

The “pharmaceutically salts” or “pharmaceutically acceptable salts” include inorganic as well as organic acids salts. Representative examples of suitable inorganic acids include hydrochloric, hydrobromic, hydroiodic, phosphoric, and the like. Representative examples of suitable organic acids include formic, acetic, trichloroacetic, trifluoroacetic, propionic, benzoic, cinnamic, citric, fumaric, maleic, methanesulfonic and the like. Further examples of pharmaceutically inorganic or organic acid addition salts include the pharmaceutically salts listed in J. Pharm. Sci. 1977, 66, 2, and in Handbook of Pharmaceutical Salts: Properties, Selection, and Use edited by P. Heinrich Stahl and Camille G. Wermuth 2002. In a preferred embodiment, the salt is selected from the group consisting of maleate, chlorhydrate, bromhydrate, and methanesulfonate. The “pharmaceutically salts” also include inorganic as well as organic base salts. Representative examples of suitable inorganic bases include sodium or potassium salt, an alkaline earth metal salt, such as a calcium or magnesium salt, or an ammonium salt. Representative examples of suitable salts with an organic base include for instance a salt with methylamine, dimethylamine, trimethylamine, piperidine, morpholine, tris-(2-hydroxyethyl)amine. The “pharmaceutically salts” or “pharmaceutically acceptable salts” further include a salt with amino acids salts such as aspartic acid, lysine, and arginine. In a preferred embodiment, the salt is selected from the group consisting of sodium, lysine, arginine salt. It also includes disodium, diarginine, and dilysine salts.

The present invention provides highly soluble water indole derivatives, which are substituted on the nitrogen of the indole core (i.e., N-substituted indole derivatives).

As used herein, derivatives or compounds which are not substituted on the nitrogen of the indole core are also called with no limitation, “indole derivative(s)”, “payload”, “active ingredient”, “active compound”, “pharmaceutical active ingredient”, “biological active ingredient”, “biological active form”, “drug”, or “molecule”. Such “indole derivative(s)” and the like have the following formula:

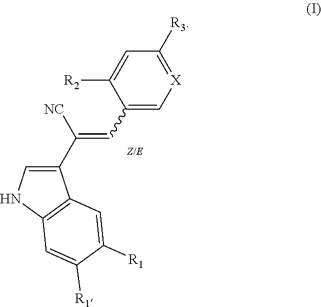

in which X, R₁, Rr, R₂, and R₃ are defined in the present disclosure.

As used herein, N-substituted indole derivatives are also called “prodrugs”.

In general, “prodrugs” as used herein are “N-substituted indole derivatives” of formula (I), which themselves may be less active or even inactive, but which, upon administration, are converted to the corresponding biological active form under physiological conditions, for example by metabolism, solvolysis, or otherwise.

Particularly, prodrugs of an indole derivative as defined herein may be compounds of formula (I) obtained by subjecting the amino group of the indole derivative to an acylation, alkylation or phosphorylation (e.g., a compound of formula (I) obtained by subjecting the amino group of the indole derivative to an eicosanoylation, an alanylation, a pentylaminocarbonylation, a (5-methyl-2-oxo-1,3-dioxolen-4-yl) methoxycarbonylation, a tetrahydrofuranylation, a pyrrolidylmethylation, a pivaloyloxymethylation and a tert-butylation).

In an embodiment, prodrugs of an indole derivative as defined herein include, without limitation, any prodrugs suitable to drugs comprising an amino group, such as indole derivatives drugs. For instance, it may include prodrugs such as amides, carbamates and thiocarbamates (O-thiocarbamates or S-thiocarbamates), N-acyloxyalkyl derivatives, N-acyloxylakoxy carbonyl derivatives, β-aminoketones, (oxodioxolenyl)methyl derivatives, N-Mannich bases, imines (Schiff bases), enamines and enaminones, azo compounds, lactonization systems, tetrahydro-(2H)-1,3,5-thiadiazine-2-thione (THTT), redox systems, and PEG as disclosed by Simplicio et al. (Molecules 2008, 13, 519-547).

In an embodiment, prodrugs of an indole derivative as defined herein are an amide prodrug type. More specifically, the nitrogen atom of the indole core is substituted by a —CO—R′ group with R′ can represent H, a (C₁-C₆)alkyl optionally substituted by a halogen, a cycloalkyl, an aryl or a heteroaryl, which may be substituted or unsubstituted. In a further embodiment, prodrugs of an indole derivative as defined herein are a carbamate prodrug type. More specifically, the nitrogen atom of the indole core is substituted by a —CO₂—R′ group with R′ can represent H, a (C₁-C₆)alkyl optionally substituted by a halogen, a cycloalkyl, an aryl or a heteroaryl, which may be substituted or unsubstituted. In a further embodiment, prodrugs of an indole derivative as defined herein are a N-acyloxyalkylamine. More specifically, the nitrogen atom of the indole core is substituted by a —CH(R″)—CO₂—R′ group with R′ can represent H, a (C₁-C₆)alkyl optionally substituted by a halogen, a cycloalkyl, an aryl or a heteroaryl, which may be substituted or unsubstituted, and R″ can represent H, a (C₁-C₆)alkyl optionally substituted by a halogen. The amide, carbamate, and N-acyloxyalkylamine prodrug types can release the drug by an amidase, a peptidase, or an esterase.

In an embodiment, prodrugs of an indole derivative as defined herein are a sulphenamide prodrug type. More specifically, the nitrogen atom of the indole core is substituted by a —S—R′ group with R′ can represent H, a (C₁-C₆)alkyl optionally substituted by a halogen, a cycloalkyl, an aryl or a heteroaryl, which may be substituted or unsubstituted.

In an embodiment, prodrugs of an indole derivative as defined herein are a phosphate prodrug type. More specifically, the nitrogen atom of the indole core is substituted by a (C₁-C₆)alkyl-O—P(O)(OH)(O⁻) group, a —NH—P(O)—(OH)₂ group, a —N—P(O)—(OH) group, a —N—P—(OH)₂ group or a —NO—P(O)—(OH)₂. The phosphate prodrug types can release the drug in the presence of a phosphatase, such as an alkaline phosphatase.

In an embodiment, prodrugs of an indole derivative as defined herein are N-Mannich base prodrug type. More specifically, the nitrogen atom of the indole core is substituted by a (C₁-C₆)alkyl-NH—CO—R′ group with R′ can represent H, a (C₁-C₆)alkyl optionally substituted by a halogen, a cycloalkyl, an aryl or a heteroaryl, which may be substituted or unsubstituted.

In a particular embodiment, prodrugs of an indole derivative as defined herein is selected from the group consisting of:

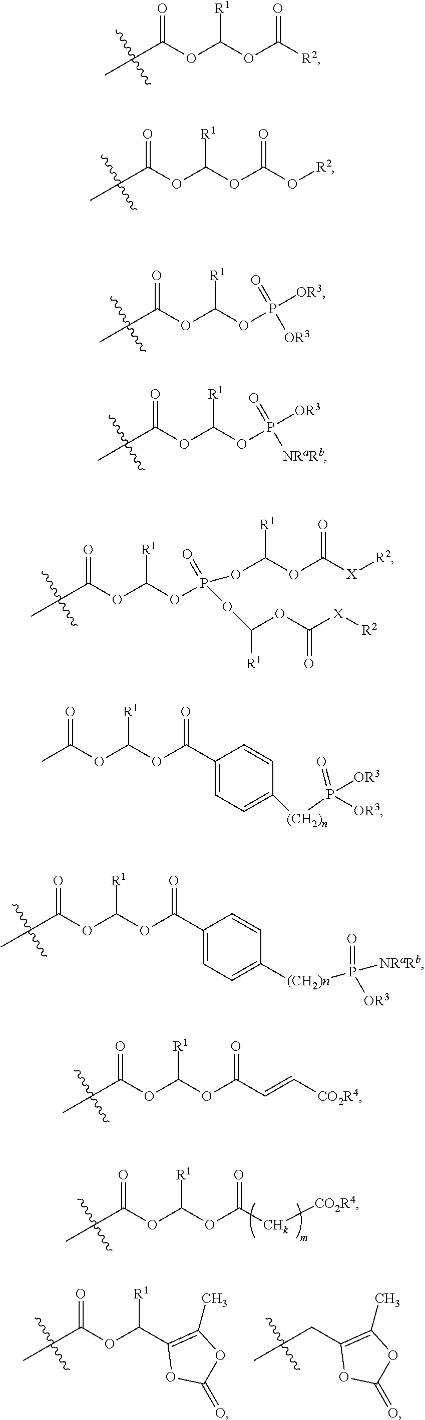

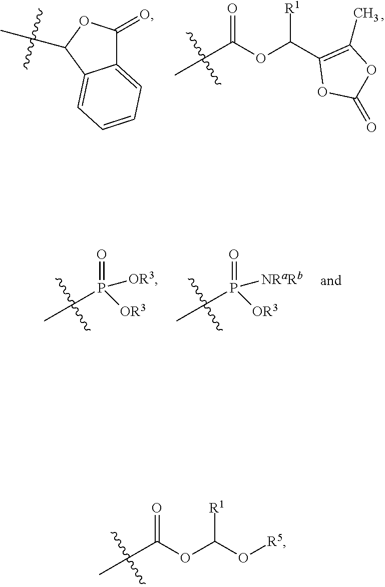

where R₁ may be H, alkyl or particularly C₁-C₈ alkyl;
R₂ may be alkyl, cycloalkyl, aryl, or heteroaryl, or halo alkyl, which may be substituted or unsubstituted;
R₃ may be H, metal, R₂ or a substituted or unsubstituted primary, secondary or tertiary amine;
R₄ may be H, metal, ammonium salt, or alkyl;
R₅ may be a substituted or unsubstituted natural amino acid;
R^{a} or R^{b} may be H, alkyl or aryl; or NR^{a} or NR^{b} may be an amino acid;
X may be C or O;
k may be 1 or 2, m may be 2-22, or (CHₖ)ₘ may be saturated, unsaturated or conjugated hydrocarbon;
n may be 0-2; and
the metal may be Na, K, Li, Ca, Mg, Ag or Zn.

In a further particular embodiment, prodrugs of an indole derivative as defined herein are a phosphothiolate prodrug type. More specifically, the nitrogen atom of the indole core is substituted by a (C₁-C₆)alkyl-NH—P(O)(OR′)—C(V)—X—S—R″ or by a —P(O)(OR′)—C(V)—X—S—R″ where:
V represents (C₁-C₈)alkyl;
X represents CR3 (R₄);
R′ independently represents an optionally substituted aliphatic or aromatic residue; optionally, R′ represents (C₁-C₈)alkyl optionally substituted with at least one of (C₁-C₈)alkoxy), wherein n is 1, 2, 3, 4, 5 or 6, F, Cl, Br, I, —NO₂, N(C₁-C₈)alkyl, —NH₂, —N((C₁-C₈)alkyl)₂, ═O, C₃-C₈-cycloalkyl, —S—S—(C₁-C₈)alkyl, hydroxy-((C₁-C₈)alkoxy), wherein n is 1, 2, 3, 4, 5 or 6, C₂-C₈-alkynyl or optionally substituted phenyl; or optionally R′ represents phenyl optionally independently substituted with at least one of (C₁-C₈)alkyl, (C₁-C₈)alkoxy)ₙ wherein
n is 1, 2, 3, 4, 5 or 6, F, Cl, I, Br, NO₂, N(C₁-C₈)alkyl, NH₂, or —N((C₁-C₈)alkyl) 2; or optionally,
R′ represents a 5- or 6-membered heteroaromatic system;
R₃ represents H or (C₁-C₈)alkyl;
R₄ represents H or (C₁-C₈)alkyl.
R″ represents an optionally substituted (C₁-C₈)alkyl, an optionally substituted phenyl, an optionally substituted aromatic 5- or 6-membered heterocyclic system, an amino acid, a peptide, a protein, an antibody, a saccharide, a polysaccharide, a nucleotide, an oligonucleotide or a polymer.

The phosphothiolate prodrug type is developed by the company Tubulis and more specifically disclosed in the patent U.S. Pat. No. 11,161,873 B2 and the patent application US 2020/0390901 A1.

In a further particular embodiment, prodrugs of an indole derivative as defined herein are carbohydrate prodrug type. The carbohydrate prodrug types can release the drug in the presence of a glycosidase enzyme. Particularly, carbohydrate prodrug type includes prodrugs disclosed by H. Martin, and al. (Chem. Soc. Rev., 2022, 51, 9694) or in the application No. US 2009/0227617. In a particular embodiment, it includes glycoconjugates having the following formula:

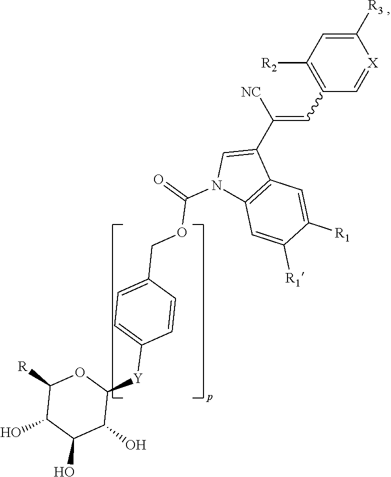

in which:
X, R₁, R_{1′}, R₂, and R₃ are defined in the present disclosure,
Y is —O— or —NH—CO—O—,
R is H, —OH, —CH₂OH, —CH₃, —COOR′ with R′ being H, —CH₃, —CH₂—CH—CH₂, —(CH₂)ₓ≡CH, —CH₂—CH₂—OH, —CH₂—CH₂—OCH₃, or a residue having formula

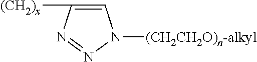

n is an integer from 1 to 40, preferably from 1 to 10, more preferably from 2 to 6;
x is an integer from 1 to 5, preferably from 1 to 3, more preferably 1 or 2;
p is an integer from 1 to 5, preferably from 1 to 3, more preferably 1 or 2;
and alkyl is a straight or branched alkyl residue having 1-10 carbon atoms, preferably 1-3 carbon atoms.

In a further particular embodiment, it includes glycoconjugates having the following formula:

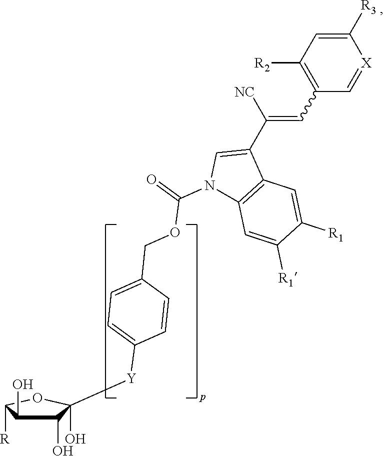

in which:
X, R₁, R_{1′}, R₂, and R₃ are defined in the present disclosure,
Y is —O— or —NH—CO—O—,
R is H, —OH, —CH₂OH, —CH₃, —COOR′ with R′ being H, —CH₃, —CH₂—CH—CH₂, —(CH₂)ₓ≡CH, —CH₂—CH₂—OH, —CH₂—CH₂—OCH₃, or a residue having formula

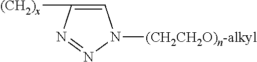

n is an integer from 1 to 40, preferably from 1 to 10, more preferably from 2 to 6;
x is an integer from 1 to 5, preferably from 1 to 3, more preferably 1 or 2;
p is an integer from 1 to 5, preferably from 1 to 3, more preferably 1 or 2;
and alkyl is a straight or branched alkyl residue having 1-10 carbon atoms, preferably 1-3 carbon atoms.

In a further embodiment according to the invention, the term “N-substituted indole derivatives” corresponds to indole derivatives in which the nitrogen atom of the indole core is substituted by a substituent Ra.

The present invention thus provides a compound or one of its pharmaceutically acceptable salts of formula (I):

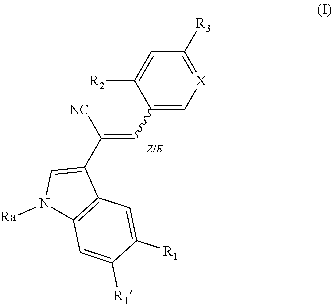

wherein:
X represents a nitrogen atom, a C—CN unit or a N+—O-unit;
R₁ and R₁′ represent independently a hydrogen, a halogen, a (C₁-C₆)alkoxy, or a —SO₂—CH₃ group, with the proviso that if R₁ or R₁′ is a (C₁-C₆)alkoxy, then R² is not a halogen;
R₂ represents:
   a radical (C₁-C₆)alkoxy, (C₃-C₆)cycloalkoxy, (C₃-C₆)heterocycloalkoxy, aryloxy, heteroaryloxy, (C₁-C₆)alkyl-aryloxy, (C₁-C₆)alkyl-heteroaryloxy, said radicals being optionally substituted by at least one halogen, or a radical thio-(C₁-C₆)alkyl, thio-aryl, thio-heteroaryl, thio-(C₁-C₆)alkyl-aryl or thio-(C₁-C₆)-alkyl-heteroaryl, said radicals being optionally substituted by at least one halogen or by a (C₁-C₆)alkoxy group,
   a —NR₄R₅ unit, a O—(C₁-C₆)alkyl-NR₄R₅ unit or a S—(C₁-C₆)alkyl-NR₄R₅ unit wherein R⁴ and R₅ represent H, a (C₁-C₆)alkyl group, or R₄ and R⁵ taken together form a 3- to 7-membered ring, optionally interrupted by one or several heteroatoms, with the proviso that at least one among R₄ and R₅ is not H,
   a NHCOR₆ unit wherein R⁶ represents (C₁-C₆)alkyl group,
   an aryl or a heteroaryl group optionally substituted by at least one halogen, a trifluoromethyl group, or a (C₁-C₃)alkoxy group, or
   a halogen,
R₃ represents a hydrogen, a (C₁-C₃)alkyl group, a (C₁-C₃)alkoxy group or a halogen; and
Ra is a radical selected in a group consisting of:
   a group of formula (A):

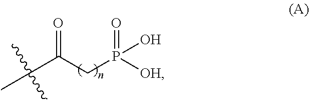

or formula (A′):

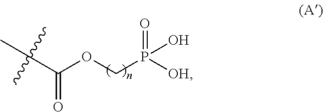

in which n is an integer comprised between 1 and 12; and
a group of formula (B):

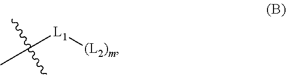

in which:
   L₁ is a cleavable group chosen among a pH-sensitive group, a photo-induced cleavable group, a bioreductible cleavable group, and an enzymatic cleavable group;
   L₂ is a tert-butoxycarbonyl group or a binding protein connector, and
   m is an integer equal to 0 or 1;
or a pharmaceutically acceptable salt thereof.

### Indole Derivatives and the Like (“Payload”)

In a particular embodiment, the indole derivatives and the compounds of formula (I) are (Z)-isomers. In a further particular embodiment, the indoles derivatives and the compounds of formula (I) are (E)-isomers.

According to the invention, R₁ and R₁′ represent independently a hydrogen, a halogen, a (C₁-C₆)alkoxy, or a —SO₂—CH₃ group, with the proviso that if R₁ or R₁′ is a (C₁-C₆)alkoxy, then R² is not a halogen. In a particular embodiment, R₁ and R₁′ are such that one is H and the other represents a halogen, a (C₁-C₆)alkoxy, or a —SO₂—CH₃ group, with the proviso that if R₁ or R₁′ is a (C₁-C₆)alkoxy, then R² is not a halogen. Preferably, R₁ and R₁′ are such that one is H and the other represents a halogen, typically, a bromine, a chlorine or a fluorine, advantageously a bromine or a chlorine, more specifically a bromine.

According to the invention, R₂ represents:
a radical (C₁-C₆)alkoxy, (C₃-C₆)cycloalkoxy, (C₃-C₆)heterocycloalkoxy, aryloxy, heteroaryloxy, (C₁-C₆)alkyl-aryloxy, (C₁-C₆)alkyl-heteroaryloxy, said radicals being optionally substituted by at least one halogen, or a radical thio-(C₁-C₆)alkyl, thio-aryl, thio-heteroaryl, thio-(C₁-C₆)alkyl-aryl or thio-(C₁-C₆)-alkyl-heteroaryl, said radicals being optionally substituted by at least one halogen or by a (C₁-C₆)alkoxy group,
a —NR₄R₅ unit, a O—(C₁-C₆)alkyl-NR₄R₅ unit or a S—(C₁-C₆)alkyl-NR₄R₅ unit wherein R⁴ and R₅ represent H, a (C₁-C₆)alkyl group, or R₄ and R⁵ taken together form a 3- to 7-membered ring, optionally interrupted by one or several heteroatoms, with the proviso that at least one among R₄ and R⁵ is not H,
a NHCOR₆ unit wherein R⁶ represents (C₁-C₆)alkyl group,
an aryl or a heteroaryl group optionally substituted by at least one halogen, a trifluoromethyl group, or a (C₁-C₃)alkoxy group, or
a halogen.

Particularly, R₂ represents:
a radical (C₁-C₆)alkoxy, phenoxy or oxetanoxy, said radicals being optionally substituted by at least one halogen, preferably a bromine, a chlorine or a fluorine, more preferably a fluorine, such as a trifluoromethyl;
a halogen, preferably a bromine, a chlorine, or a fluorine, more preferably a bromine or a chlorine;
a R₄—N—R₅ unit or a S—(C₁-C₆)alkyl-NR₄R₅ unit, wherein R⁴ and R⁵ represent H or a (C₁-C₆)alkyl group, with the proviso that at least one among R₄ and R₅ is not H,
a NHCOR₆ unit wherein R⁶ represents (C₁-C₆)alkyl group, advantageously a methyl, an ethyl or a tert-butyl; or
an aryl group optionally substituted by at least one halogen, or a trifluoromethyl group; or
a heteroaryl group, advantageously a furan, a triazol, a pyridin, a thiazol, a pyran, a pyrrol, an imidazol, a tetrazol, a benzofuran, triazinyl, pyrazinyl, a pyridazin, or a tetrazol.

Preferably, R₂ represents:
a radical (C₁-C₆)alkoxy selected from the group consisting of a methoxy group, an ethoxy group, an isopropoxy group, preferably selected from the group consisting of a methoxy group, and an isopropoxy group, or a phenoxy group, optionally substituted by a fluorine, such as a trifluoromethyl, or an oxetanoxy;
a halogen selected from the group consisting of a fluorine and a chlorine,
a R₄—N—R₅ unit or a S—(C₁-C₆)alkyl-NR₄R₅ unit wherein R⁴ and R₅ represent a methyl or an ethyl group:
a radical selected from the group consisting of a thio-methyl group, a thio-ethyl group, a thio-benzyl group, a thio-pyridinyl group and a thio-phenyl group, optionally substituted by at least one fluorine or a trifluoromethyl group;
a phenyl group optionally substituted by at least one bromine or a trifluoromethyl group; or
a heteroaryl group selected from the group consisting of a furan or a triazol.

More preferably, R₂ represents:
a radical (C₁-C₆)alkoxy, preferably a methoxy group,
a radical (C₃-C₆) heterocycloalkoxy, preferably an oxetanoxy group, or
a heteroaryl group, preferably a furanyl or a triazolyl group.

According to the invention, R₃ represents a hydrogen, a (C₁-C₃)alkyl group, preferably a methyl, an ethyl or an isopropyl, a (C₁-C₃)alkoxy group, preferably a methoxy, an ethoxy or an isopropoxy, or a halogen, advantageously a fluorine. Preferably, R₃ is a hydrogen, a methoxy or a fluorine. More preferably, R₃ is a hydrogen.

In a preferred embodiment:
X represents a C—CN unit,
R₁ represents a halogen, preferably a bromine,
R₁′ represents a hydrogen,
R₂ represents a radical (C₁-C₆)alkoxy, preferably a methoxy group, and
R₃ represents a hydrogen.

In a further preferred embodiment:
X represents a C—CN unit,
R₁ represents a hydrogen,
R₁′ represents a radical (C₁-C₆)alkoxy, preferably a methoxy group,
R₂ represents a radical (C₁-C₆)alkoxy, preferably a methoxy group, and
R₃ represents hydrogen.

In a particular aspect, indole derivatives correspond to indole derivatives disclosed in WO 2014/086964, and, more specifically, to indole derivatives in a scope of grant claims of EP 2 928 864 B1.

In a preferred embodiment, the indole derivative and the like is selected in the group consisting of:
(Z)-2-(5-chloro-1H-indol-3-yl)-3-(4-chloropyridin-3-yl)-acrylonitrile;
(Z)-2-(5-bromo-1H-indol-3-yl)-3-(4-chloropyridin-3-yl)-acrylonitrile;
(Z)-2-(5-bromo-1H-indol-3-yl)-3-(4-methoxypyridin-3-yl)-acrylonitrile;
(E)-2-(5-bromo-1H-indol-3-yl)-3-(4-methoxypyridin-3-yl)-acrylonitrile;
(Z)-2-(5-chloro-1H-indol-3-yl)-3-(4-(dimethylamino)pyridin-3-yl)-acrylonitrile;
(Z)-2-(5-chloro-1H-indol-3-yl)-3-(4-dimethylamino)pyridine-3-yl)-acrylonitrile, hydrochloride;
(Z)-2-(5-bromo-1H-indol-3-yl)-3-(4-(dimethylamino)pyridin-3-yl)-acrylonitrile;
(Z)-2-(5-chloro-1H-indol-3-yl)-3-(4-methoxypyridin-3-yl)-acrylonitrile;
(E)-2-(5-chloro-1H-indol-3-yl)-3-(4-methoxypyridin-3-yl)-acrylonitrile;
(Z)-2-(5-chloro-1H-indol-3-yl)-3-(4-phenoxypyridin-3-yl)-acrylonitrile;
(Z)-2-(5-bromo-1H-indol-3-yl)-3-(4-phenoxypyridin-3-yl)-acrylonitrile;
(Z)-2-(5-methoxy-1H-indol-3-yl)-3-(4-methoxypyridin-3-yl)-acrylonitrile;
(Z)-2-(5-bromo-1H-indol-3-yl)-3-(4-ethoxypyridin-3-yl)-acrylonitrile;
(Z)-2-(5-bromo-1H-indol-3-yl)-3-(4-isopropoxypyridin-3-yl)-acrylonitrile;
(Z)-2-(5-bromo-1H-indol-3-yl)-3-(4-(methylthio)pyridin-3-yl)-acrylonitrile;
(Z)-2-(5-bromo-1H-indol-3-yl)-3-(4-(ethylthio)pyridin-3-yl)-acrylonitrile;
(Z)-2-(5-bromo-1H-indol-3-yl)-3-(4-(3-bromophenyl)pyridin-3-yl)-acrylonitrile;
(Z)-3-(4-(3-bromophenyl)pyridin-3-yl)-2-(5-chloro-1H-indol-3-yl)-acrylonitrile;
(Z)-2-(5-bromo-1H-indol-3-yl)-3-(4-(phenylthio)pyridin-3-yl)-acrylonitrile;
(Z)-3-(4-(benzylthio)pyridin-3-yl)-2-(5-bromo-1H-indol-3-yl)-acrylonitrile;
(Z)-2-(5-bromo-1H-indol-3-yl)-3-(4-(3,4-dimethoxy)thio)pyridin-3-yl)-acrylonitrile;
(Z)-2-(5-bromo-1H-indol-3-yl)-3-(4-(4-fluorophenoxy)pyridin-3-yl)-acrylonitrile;
(Z)-2-(5-chloro-1H-indol-3-yl)-3-(4-(4-fluorophenoxy)pyridin-3-yl)-acrylonitrile;
(Z)-2-(5-bromo-1H-indol-3-yl)-3-(4-(diethylamino)pyridin-3-yl)-acrylonitrile;
(Z)-2-(5-bromo-1H-indol-3-yl)-3-(4-(4-(trifluoromethyl)phenyl)pyridin-3-yl)-acrylonitrile;
(Z)-2-(5-chloro-1H-indol-3-yl)-3-(4-(4-(trifluoromethyl)phenyl)pyridin-3-yl)-acrylonitrile;
(Z)-2-(5-chloro-1H-indol-3-yl)-3-(4-((4-fluorophenyl)thio)pyridin-3-yl)-acrylonitrile;
(Z)-2-(5-bromo-1H-indol-3-yl)-3-(4-((4-fluorophenyl)thio)pyridin-3-yl)-acrylonitrile;
(Z)-2-(5-chloro-1H-indol-3-yl)-3-(4-(furan-3-yl)pyridin-3-yl)-acrylonitrile;
(Z)-2-(5-chloro-1H-indol-3-yl)-3-(4-(pyridine-2-ylthio)pyridin-3-yl)-acrylonitrile;
(Z)-2-(5-bromo-1H-indol-3-yl)-3-(4-(pyridine-2-ylthio)pyridin-3-yl)-acrylonitrile;
(Z)-3-(4-(1H-1,2,4-triazol-1-yl) pyridin-3-yl)-2-(5-bromo-1H-indol-3-yl)-acrylonitrile;
(Z)-3-(4-(1H-1,2,4-triazol-1-yl) pyridin-3-yl)-2-(5-chloro-1H-indol-3-yl)-acrylonitrile;
(Z)-2-(5-bromo-1H-indol-3-yl)-3-(4-(furan-3-yl) pyridin-3-yl)-acrylonitrile;
(E)-2-(5-bromo-1H-indol-3-yl)-3-(4-(furan-3-yl) pyridin-3-yl)-acrylonitrile;
(Z)-2-(5-chloro-1H-indol-3-yl)-3-(4-methoxypyridin-3-yl)-acrylonitrile;
(Z)-2-(5-bromo-1H-indol-3-yl)-3-(4-(2-(dimethylamino)ethylthio)pyridin-3-yl)-acrylonitrile;
(Z)-3-(2-(5-bromo-1H-indol-3-yl)-2-cyanovinyl)-4-(4-fluorophenoxy)benzonitrile;
(Z)-3-(2-(5-bromo-1H-indol-3-yl)-2-cyanovinyl)-4-methoxybenzonitrile;
(E)-3-(2-(5-bromo-1H-indol-3-yl)-2-cyanovinyl)-4-methoxybenzonitrile;
(Z)-3-(2-(5-bromo-1H-indol-3-yl)-2-cyanovinyl)-4-(dimethylamino)benzonitrile;
(Z)-3-(2-(5-chloro-1H-indol-3-yl)-2-cyanovinyl)-4-methoxybenzonitrile;
(Z)-3-(2-(5-chloro-1H-indol-3-yl)-2-cyanovinyl)-4-(dimethylamino)benzonitrile;
(Z)-3-(2-(5-chloro-1H-indol-3-yl)-2-cyanovinyl)-4-(ethylthio)benzonitrile;
(Z)-2-(6-bromo-1H-indol-3-yl)-3-(4-methoxypyridin-3-yl) acrylonitrile;
(Z)-2-(6-fluoro-1H-indol-3-yl)-3-(4-methoxypyridin-3-yl) acrylonitrile;
(Z)-2-(6-chloro-1H-indol-3-yl)-3-(4-methoxypyridin-3-yl) acrylonitrile;
(Z)-3-(2-(5-bromo-1H-indol-3-yl)-2-cyanovinyl)-4-(furan-3-yl)pyridine-1-oxide;
(Z)-3-(2-(5-chloro-1H-indol-3-yl)-2-cyanovinyl)-4-methoxypyridine-1-oxide;
(Z)-3-(2-(5-chloro-1H-indol-3-yl)-2-cyanovinyl)-4-(trifluoromethoxy)benzonitrile;
(Z)-3-(2-(5-bromo-1H-indol-3-yl)-2-cyanovinyl)-4-(trifluoromethoxy)benzonitrile; and their pharmaceutically acceptable salts.

In a more preferred embodiment, the indole derivative is the example 38 of WO 2014/086964: (Z)-3-(2-(5-bromo-1H-indol-3-yl)-2-cyanovinyl)-4-methoxybenzonitrile having the following formula:

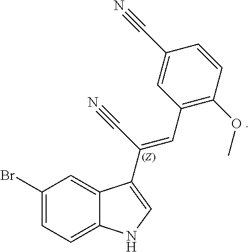 [SMILES: COc1ccc(C#N)cc1/C=C(\C#N)c1c[nH]c2ccc(Br)cc12]

### Phosphonic Series

The present invention provides compounds of formula (I) as “phosphonic prodrugs” of indoles derivatives as above defined.

Thus, in a particular embodiment, Ra is a group of formula (A):

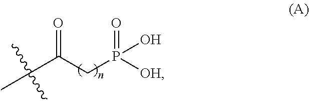

formula (A′):

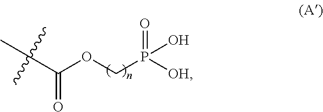

in which n is an integer comprised between 1 and 12. In a preferred embodiment, n is an integer comprised between 2 and 10, more preferably between 2 and 8, and even more preferably between 2 and 6. Advantageously, n is 2, 3, 4, 5, or 6.

In a particular embodiment, “phosphonic prodrugs” include their pharmaceutically acceptable salts. Preferably, the pharmaceutically acceptable salt is chosen among a sodium salt, a disodium salt, a lysine salt, a dilysine salt, an arginine salt or a diarginine salt. More preferably, the pharmaceutically acceptable salt is chosen among a sodium salt or a disodium salt, even more preferably is a sodium salt.

In a preferred embodiment, the compound of formula (I) is selected from the group consisting of:
Example 1: (Z)-5-(5-bromo-3-(1-cyano-2-(5-cyano-2-methoxyphenyl)vinyl)-1H-indol-1-yl)-5-oxopentylphosphonic acid;
Example 2: (Z)-3-(5-bromo-3-(1-cyano-2-(5-cyano-2-methoxyphenyl)vinyl)-1H-indol-1-yl)-3-oxopropylphosphonic acid;
Example 3: (Z)-7-(5-bromo-3-(1-cyano-2-(5-cyano-2-methoxyphenyl)vinyl)-1H-indol-1-yl)-7-oxoheptylphosphonic acid;
Example 4: (Z)-5-(3-(1-cyano-2-(5-cyano-2-methoxyphenyl)vinyl)-6-methoxy-1H-indol-1-yl)-5-oxopentylphosphonic acid;
Example 5: (Z)-7-(3-(1-cyano-2-(5-cyano-2-methoxyphenyl)vinyl)-6-methoxy-1H-indol-1-yl)-7-oxoheptylphosphonic acid;
Example 6: Sodium (Z)-hydrogen5-(5-bromo-3-(1-cyano-2-(5-cyano-2-methoxyphenyl)vinyl)-1H-indol-1-yl)-5-oxopentylphosphonate;
Example 7 sodium (Z)-hydrogen3-(5-bromo-3-(1-cyano-2-(5-cyano-2-methoxyphenyl)vinyl)-1H-indol-1-yl)-3-oxopropylphosphonate;
Example 8: sodium (Z)-hydrogen4-(5-bromo-3-(1-cyano-2-(5-cyano-2-methoxyphenyl)vinyl)-1H-indol-1-yl)-4-oxobutylphosphonate;
Example 9: sodium (Z)-hydrogen6-(5-bromo-3-(1-cyano-2-(5-cyano-2-methoxyphenyl)vinyl)-1H-indol-1-yl)-4-oxohexylphosphonate;
Example 10: sodium (Z)-hydrogen7-(5-bromo-3-(1-cyano-2-(5-cyano-2-methoxyphenyl)vinyl)-1H-indol-1-yl)-7-oxoheptylphosphonate;
Example 11: sodium (Z)-hydrogen5-(3-(1-cyano-2-(5-cyano-2-methoxyphenyl)vinyl)-6-methoxy-1H-indol-1-yl)-5-oxopentylphosphonate;
Example 12: sodium (Z)-hydrogen7-(3-(1-cyano-2-(5-cyano-2-methoxyphenyl)vinyl)-6-methoxy-1H-indol-1-yl)-7-oxoheptylphosphonate;
Example 13: disodium (Z)-4-(5-bromo-3-(1-cyano-2-(5-cyano-2-methoxyphenyl)vinyl)-1H-indol-1-yl)-4-oxobutylphosphonate;
Example 14: disodium (Z)-5-(5-bromo-3-(1-cyano-2-(5-cyano-2-methoxyphenyl)vinyl)-1H-indol-1-yl)-5-oxopentylphosphonate;
Example 15: (S)-2,6-diaminohexanoic acid compound with (Z)-3-(5-bromo-3-(1-cyano-2-(5-cyano-2-methoxyphenyl)vinyl)-1H-indol-1-yl)-3-oxopropylphosphonic acid (2:1);
Example 16: (S)-2,6-diaminohexanoic acid compound with (Z)-4-(5-bromo-3-(1-cyano-2-(5-cyano-2-methoxyphenyl)vinyl)-1H-indol-1-yl)-4-oxobutylphosphonic acid (2:1);
Example 17:3-[hydroxy(dioxo)-lambda6-phosphanyl]propyl 5-bromo-3-[(Z)-1-cyano-2-(2-cyano-5-methoxy-phenyl)vinyl]indole-1-carboxylate; and
Example 18:4-[hydroxy(dioxo)-lambda6-phosphanyl]butyl 5-bromo-3-[(Z)-1-cyano-2-(2-cyano-5-methoxy-phenyl)vinyl]indole-1-carboxylate.

### L₁ Series

The present invention further provides compounds of formula (I) as “L₁ prodrugs” of indoles derivatives as above defined. As used herein, “L₁ prodrugs” correspond to prodrugs comprising a cleavable group L₁.

Thus, in a particular embodiment, Ra is a group of formula (B):

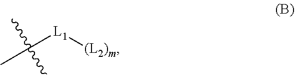

in which:
L₁ is a cleavable group chosen among a pH-sensitive group, a photo-induced cleavable group, a bioreductible cleavable group, and an enzymatic cleavable group;
L₂ is a tert-butoxycarbonyl group or a binding protein connector, and
m is an integer equal to 0 or 1.

According to the invention, L₁ is a cleavable group chosen among a pH-sensitive group, a photo-induced cleavable group, a reductive cleavable group, and an enzymatic cleavable group.

In a particular embodiment, L₁ is a pH-sensitive group. As used herein, a “pH-sensitive” group is sensitive to hydrolysis and may be cleaved at certain pH values, particularly under acidic conditions at a pH range of 4 to 6. Accordingly, such a pH-sensitive group may be an acid-induced cleavable group or an acid-labile group, which is relatively stable in the neutral environment of blood (pH 7.3-7.5) and can be hydrolyzed in slightly acidic endosomes (pH 5.0-6.5) and lysosomes (pH 4.5-5.0). As an example of pH-sensitive group hydrolysable, it may be cited, without limitation, hydrazine, hydrazone, semicarbazone, thiosemicarbazone, cis-aconitic amide, orthoester, carbonate, acetal, ketal, boronate ester, and the like. In a preferred embodiment, a pH sensitive group as above defined is an acid-induced cleavable group.

In a further particular embodiment, L₁ is a photo-induced cleavable group.

In a further particular embodiment, L₁ is a bioreductible cleavable group. As used herein, a “bioreductible cleavable” group is cleavable under biologic reducing conditions. For instance, the group is cleavable in the presence of a reducing agent, such as glutathione or dithiothreitol. A glutathione-sensitive group may thus be cleaved by a disulfide exchange reaction with a glutathione species inside a cell. Preferably L₁ is a glutathione-sensitive group or a group having a disulfide, or a group having a sulfonamide. A variety of disulfide groups are known in the art, including for example, those that can be formed using SATA (N-succinimidyl-5-acetylthioacetate), SPDP (N-succinimidyl-3-(2-pyridyldithio) propionate), SPDB (N-succinimidyl-3-(2-pyridyldithio) butyrate) and SMPT (N-succinimidyloxycarbonyl-alpha-methyl-alpha-(2-pyridyl-dithio) toluene). As used herein, a sulfonamide group is a sulfonyl group connected to an amine group, in which the sulfur-nitrogen bond can be cleaved.

In a further particular embodiment, L₁ is an enzymatic cleavable group. As used herein, an “enzymatic cleavable” group is a group cleaved by an enzyme, such as a protease, a peptidase, an esterase, a beta-glucuronidase, a glycosidase, a phosphodiesterase, a phosphatase, a pyrophosphatase, a tubulin-tyrosine ligase or a lipase.

In an embodiment, the enzymatic cleavable group L₁ is cleaved by a protease or a peptidase.

Examples of proteases include, but are not limited to, cathepsin B, VAGP tetrapeptide, and the like. Preferably, the enzymatic cleavable group L₁ is cleaved by a peptidase. In this embodiment, the enzymatic cleavable group L₁ contains a peptide, which is the site of cleavage of the linker, thereby facilitating release of the drug upon exposure to intracellular proteases, such as lysosomal enzymes. Peptides can be designed and optimized for enzymatic cleavage by a particular enzyme, for example, a tumor-associated protease, cathepsin B, C and D, or a plasmin protease. Examples of peptides having two amino acids include, but are not limited to, alanine-alanine (ala-ala), valine-alanine (val-ala), valine-citrulline (vc or val-cit), valine-arginine (val-arg), valine-lysine (val-lys), alanine-phenylalanine (af or ala-phe); phenylalanine-lysine (fk or phe-lys); phenylalanine-homolysine (phe-homolys); and N-methyl-valine-citrulline (Me-val-cit). Examples of peptides having three amino acids include, but are not limited to, glycine-valine-citrulline (gly-val-cit), aspartic acid-valine-citrulline (asp-val-cit), alanine-alanine-asparagine (ala-ala-asn), alanine-phenylalanine-lysine (ala-phe-lys), glutamic acid-cysteine-glycine (glu-cys-gly), glycine-glycine-phenylalanine (gly-gly-phe), and glycine-glycine-glycine (gly-gly-gly). Examples of peptides having four amino acids include, but are not limited to, glycine-glycine-valine-citrulline (gly-gly-val-cit), glycine-glycine-phenylalanine-glycine (gly-gly-phe-gly) and glutamic acid-glycine-glutamic acid-glutamic acid (glu-gly-glu-glu). The amino acid combinations above can also be present in the reverse order (i.e., cit-val).

Examples of peptides are particularly provided in Table A.

TABLE A

| | Sequence | SEQ ID |
|---|---|---|
| 1 | Gly Gly Val X | |
| 2 | X Val Gly Gly | |
| 3 | Gly Gly Phe Gly | SEQ ID NO: 1 |
| 4 | Gly Phe Gly Gly | SEQ ID NO: 2 |
| 5 | Glu Gly Glu Glu | SEQ ID NO: 3 |
| 6 | Glu Glu Gly Glu | SEQ ID NO: 4 |

X = cit or citruline

The peptides of the present disclosure can comprise L- or D-isomers of amino acid residues. The term “naturally-occurring amino acid” refers to Ala, Asp, Asx, Cit, Cys, Glu, Phe, Glx, Gly, His, lie, Lys, Leu, Met, Asn, Pro, Gin, Arg, Ser, Thr, Val, Trp, and Tyr. “D-” designates an amino acid having the “D” (dextrorotary) configuration, as opposed to the configuration in the naturally occurring (“L-”) amino acids. The amino acids described herein can be purchased commercially or synthesized using methods known in the art.

Preferably, L₁ is a p-aminobenzyloxycarbonyl-AA1_{w}-AA2ₓ-AA3_{y}-AA4_{z} group with AA1, AA2, AA3, and AA4 represent independently an amino acid as defined above, and w, x, y, and z are independently an integer equal to 0 or 1. A used herein, the group “p-aminobenzyloxycarbonyl” is also called PABC. More preferably, L₁ is a p-aminobenzyloxycarbonyl-AA1_{w}-AA2ₓ-AA3_{y}-AA4_{z} group with AA1, AA2, AA3, and AA4 represent independently an amino acid selected in a group consisting of alanine, arginine, valine, citrulline, phenylalanine, lysine, glycine, aspartic acid, asparagine, glutamic acid, and derivatives thereof, preferably citrulline, valine, cysteic acid, glycine, and glutamic acid, more preferably citrulline, valine, and cysteic acid, and w, x, y, and z are independently an integer equal to 0 or 1. Even more preferably, L₁ is group selected in the group consisting of: -PABC-Cit-Val, and -PABC-Cit-Val-Cysteic acid.

In an embodiment, the enzymatic cleavable group L₁ is cleaved by a pyrophosphatase. In this embodiment, L₁ contains a pyrophosphate group.

In an embodiment, the enzymatic cleavable group L₁ is cleaved by a beta-glucuronidase. In a preferred embodiment, L₁ is a -PABC-glucuronide group.

In an aspect, the “L₁ prodrugs” are such that L₁ is above defined and L₂ is absent. In this aspect, the compounds of formula (I) are such that Ra is a group of formula (B):

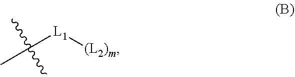

in which L₁ is such as above defined and m is an integer equal to 0.

According to this aspect, a preferred compound is a compound selected from the group consisting of:
Example 19: (2S,3S,4S,5R,6S)-6-[4-[[5-bromo-3-[(Z)-1-cyano-2-(5-cyano-2-methoxy-phenyl)vinyl]indole-1-carbonyl]oxymethyl]phenoxy]-3,4,5-trihydroxy-tetrahydropyran-2-carboxylic acid;
Example 21: [4-[[(2S)-2-[[(2S)-2-aminopropanoyl]amino]-5-ureido-pentanoyl]amino]phenyl]methyl-5-bromo-3-[(Z)-1-cyano-2-(5-cyano-2-methoxy-phenyl)vinyl]indole-1-carboxylate hydrochloride; and
Example 25: 2-amino-3-[(1S)-1-[[(1S)-1-[[4-[[5-bromo-3-[(Z)-1-cyano-2-(5-cyano-2-methoxy-phenyl)vinyl]indole-1-carbonyl]oxymethyl]phenyl]carbamoyl]-4-ureido-butyl]carbamoyl]-2-methyl-propyl]amino]-3-oxo-propane-1-sulfonic acid.

In an aspect, the “L₁ prodrugs” are such that L₁ is above defined, m is an integer equal to 1, and L₂ is a tert-butoxycarbonyl (Boc) group. In this aspect, the compounds of formula (I) are such that Ra is a group of formula (B):

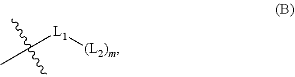

in which L₁ is such as above defined and m is an integer equal to 0.

According to this aspect, a preferred compound is a compound selected from the group consisting of:
Example 20: [4-[[(2S)-2-[[(2S)-2-(tert-butoxycarbonylamino)-3-methyl-butanoyl]amino]-5-ureido-pentanoyl]amino]phenyl]methyl-5-bromo-3-[(Z)-1-cyano-2-(5-cyano-2-methoxy-phenyl)vinyl]indole-1-carboxylate; and
Example 24: 3-[[(1S)-1-[(1S)-1-[[4-[5-bromo-3-[(Z)-1-cyano-2-(5-cyano-2-methoxy-phenyl)vinyl]indole-1-carbonyl]oxymethyl]phenyl]carbamoyl]-4-ureido-butyl]carbamoyl]-2-methyl-propyl]amino]-2-(tert-butoxycarbonylamino)-3-oxo-propane-1-sulfonic acid.

In an aspect, the “L₁ prodrugs” are such that L₁ is above defined, m is an integer equal to 1, and L₂ is a binding protein connector.

As used herein, a “binding protein connector” is a group able to react with or bound to or connected to a peptide, a protein or an antibody. More particularly, a “binding protein connector” includes any chemical moiety or group that is capable of linking a peptide, a protein or an antibody via a stable and covalent bound. For instance, a “binding protein connector” has a functionality that is capable of reacting with a free cysteine present on a peptide, a protein or an antibody.

Examples of “binding protein connectors” include, but are not limited to, SMCC (succinimidyl 4-(N-maleimidomethyl)cyclohexane-1-carboxylate) groups, succinimide thioether groups. Further examples of “binding protein connectors” include groups having the following formulae:

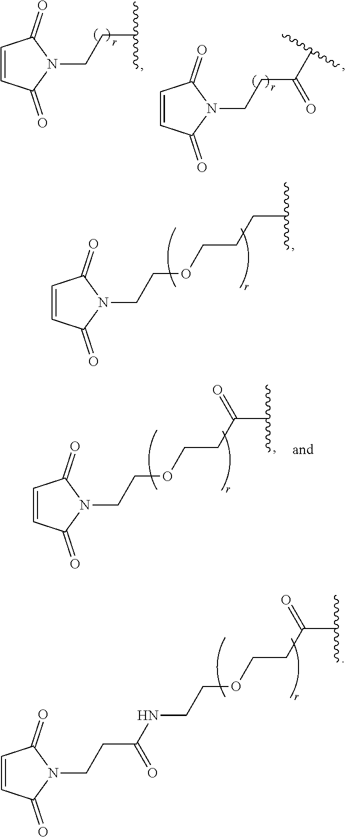

with r is an integer comprised between 1 and 36, preferably between 1 and 24, more preferably equal to 2, 4, 8, 12, 16, 20, or 24.

According to this aspect, a preferred compound is a compound selected from the group consisting of:
Example 22: [4-[[(2S)-2-[[(2S)-2-[3-[2-[3-(2,5-dioxopyrrol-1-yl)propanoylamino]ethoxy](peg4)propanoylamino]-3-methyl-butanoyl]amino]-5-ureido-pentanoylJamino]phenyl]methyl-5-bromo-3-[(Z)-1-cyano-2-(5-cyano-2-methoxy-phenyl)vinyl]indole-1-carboxylate; and
Example 26: (2R)-3-[[(1S)-1-[[(1S)-1-[[4-[[5-bromo-3-[(Z)-1-cyano-2-(5-cyano-2-methoxy-phenyl)vinyl]indole-1-carbonyl]oxymethyl]phenyl]carbamoyl]-4-ureido-butyl]carbamoyl]-2-methyl-propyl]amino]-2-[3-[2-[3-(2,5-dioxopyrrol-1-yl)propanoylamino]ethoxy]propanoylamino]-3-oxo-propane-1-sulfonic acid.

### Conjugates

The present invention also provides a conjugate of formula (II):

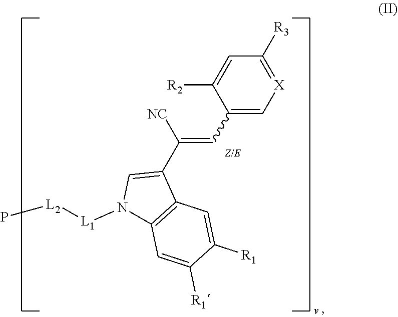

wherein:
X, R₁, R_{1′}, R₂, R³, and L₁ are such as defined herein;
L₂ is a binding protein connector;
P is a peptide or a protein, preferably an antibody, an antibody fragment, or an antigen-binding fragment, which is able to bind a target of interest; and
v is an integer from 1 to 10.

According to the invention, the conjugates of formula (II) are such as:
X, R₁, R_{1′}, R₂, R₃ are defined in the above “indole derivatives” or “payload” section including all the particular and preferred embodiments;
L₁ is a cleavable group defined in the above “L₁ series” section including all the particular and preferred embodiments; and
L₂ is a binding protein connector defined in the above “L₁ series” section including all the particular and preferred embodiments.

According to the invention, the peptide or the protein “P” can be conjugated to more than one or v compounds of formula (I) with m is 1 and L₂ is a binding protein connector. For instance, the peptide or the protein “P” can be conjugated with from 1 to 10, from 1 to 9, from 1 to 8, for example 1, 2, 3, 4, 5, 6, 7, 8, 9, or 10 compounds of formula (I) with m is 1 and L₂ is a binding protein connector. In a preferred embodiment v is an integer from 1 to 8, from 2 to 8. In a particular preferred embodiment, v is an integer equal to 1, 2, 3, 4, 5, 6, 7, or 8.

According to the invention, P is a peptide or a protein able to bind a target of interest. As used herein “peptide or a protein able to bind a target of interest” refers to any peptide and/or protein that recognizes and binds to a cell surface molecule, in particular a cell surface marker, antigen, epitope, or receptor. In a particular aspect, the protein P binds to another protein, not limited to a polypeptide moiety. The protein P targeting to a specific cell, tissue, or location, may also have certain therapeutic effect such as antiproliferative (cytostatic and/or cytotoxic) activity against a target cell or pathway. In a particular aspect, the protein P can comprise or can be engineered to comprise at least one chemically reactive group such as a carboxylic acid, amine, thiol, or chemically reactive amino acid moiety or side chain. In a particular aspect, the protein P can comprise a targeting moiety which binds or complexes with a cell surface molecule, such as a cell surface receptor or antigen, for a given target cell population. Following specific binding or complexing with the receptor, the cell is permissive for uptake of the targeting conjugate, which is then internalized into the cell.

In a particular embodiment, P is a protein, preferably albumin. These albumin-conjugates of formula (II) increase circulating half-life of the payload and promote intracellular delivery to tumors.

In a preferred embodiment, P is an antibody, an antibody fragment, or an antigen-binding fragment.

An antibody is a protein generated by the immune system that is capable of recognizing and binding to a specific antigen. A target antigen generally has numerous binding sites, also called epitopes or antigenic determinants, recognized by CDRs on multiple antibodies. Each antibody that specifically binds to a different epitope has a different structure. Thus, one antigen may have more than one corresponding antibody. The term “antibody” is used herein in the broadest sense and specifically covers monoclonal antibody, single domain antibody, polyclonal antibody, multispecific antibody (e.g., bispecific antibody), and an antibody fragment, so long as it exhibits the desired biological activity. Antibodies may be murine, human, humanized, chimeric, or derived from other species.

Monoclonal antibodies are homogeneous populations of antibodies to a particular antigenic determinant (e.g., a cancer cell antigen, a viral antigen, a microbial antigen, a protein, a peptide, a carbohydrate, a chemical, nucleic acid, or fragments thereof). A monoclonal antibody (mAb) to an antigen-of-interest can be prepared by using any technique known in the art which provides for the production of antibody molecules by continuous cell lines in culture. These include, but are not limited to, the human B cell hybridoma technique, and the EBV-hybridoma technique. Such antibodies may be of any immunoglobulin class including IgG, IgM, IgE, IgA, and IgD and any subclass thereof. The hybridoma producing the mAbs of use in this disclosure may be cultivated in vitro or in vivo.

Useful monoclonal antibodies include, but are not limited to, human monoclonal antibodies, humanized monoclonal antibodies, antibody fragments, or chimeric human-mouse (or other species) monoclonal antibodies. Human monoclonal antibodies may be made by any of numerous techniques known in the art.

In a particular aspect, the antibody can be a bispecific antibody (BsAb), i.e. a protein, typically an artificial protein, that can simultaneously bind to two different types of antigen or two different epitopes on the same antigen. The BsAb can be a bispecific monoclonal antibody (BsMAb). Methods for making bispecific antibodies are known in the art. Traditional production of full-length bispecific antibodies is based on the coexpression of two immunoglobulin heavy chain-light chain pairs, where the two chains have different specificities. The main types of manufacturing methods are quadromas, chemical conjugation, and genetic recombination, and each method results in a unique format. BsAbs are often manufactured with the quadroma or the hybridoma method.

In the quadroma procedure, because of the random assortment of immunoglobulin heavy and light chains, a potential mixture of 10 different antibody molecules are produced, of which only one has the correct bispecific structure. Purification of the correct molecule, which is usually performed using affinity chromatography steps, is rather cumbersome, and the product yields are low.

According to a different approach, antibody variable domains with the desired binding specificities (antibody-antigen combining sites) are fused to immunoglobulin constant domain sequences. The fusion may be with an immunoglobulin heavy chain constant domain, comprising at least part of the hinge, C_{H}2, and C_{H}3 regions. The first heavy-chain constant region (C_{H}1) may contain the site necessary for light chain binding, present in at least one of the fusions. Nucleic acids with sequences encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in aspects when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

Bispecific antibodies may have a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. Using such techniques, bispecific antibodies can be prepared in the treatment or prevention of disease as defined herein.

Hybrid or bifunctional antibodies can be derived either biologically, i.e., by cell fusion techniques, or chemically, especially with cross-linking agents or disulfide-bridge forming reagents, and may comprise whole antibodies or fragments thereof.

The antibody can be a functionally active fragment, derivative or analog of an antibody that immunospecifically binds to cancer cell antigens, viral antigens, or microbial antigens or other antibodies bound to tumor cells or matrix. In this regard, “functionally active” means that the fragment, derivative or analog is able to elicit anti-anti-idiotype antibodies that recognize the same antigen as the antibody from which the fragment, derivative or analog is derived. Specifically, in an exemplary aspect, the antigenicity of the idiotype of the immunoglobulin molecule can be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art.

Other useful antibodies include fragments of antibodies such as, but not limited to, F(ab′)₂ fragments, which contain the variable region, the light chain constant region and the C_{H}1 domain of the heavy chain, which can be produced by pepsin digestion of the antibody molecule, and Fab fragments, which can be generated by reducing the disulfide bridges of the F(ab′)₂ fragments. Other useful antibodies are heavy chain and light chain dimers of antibodies, or any minimal fragment thereof such as Fvs or single chain antibodies (SCAs), or any other molecule with the same specificity as the antibody. As used herein an antibody fragment is a fragment of an antibody as defined herein.

Additionally, recombinant antibodies, such as chimeric and humanized monoclonal antibodies, comprising both human and non-human portions, which can be made using standard recombinant DNA techniques, are useful antibodies. A chimeric antibody is a molecule in which different portions are derived from different animal species, such as for example those having a variable region derived from a murine monoclonal and human immunoglobulin constant regions. Humanized antibodies are antibody molecules from non-human species having one or more complementarity determining regions (CDRs) from the non-human species and a framework region from a human immunoglobulin molecule. Such chimeric and humanized monoclonal antibodies can be produced by recombinant DNA techniques known in the art.

Completely human antibodies can be produced using transgenic mice that are incapable of expressing endogenous immunoglobulin heavy and light chains genes, but which can express human heavy and light chain genes. The transgenic mice are immunized in the normal fashion with a selected antigen, e.g., all or a portion of a polypeptide of the disclosure. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). Other human antibodies can be obtained commercially from, for example, Abgenix, Inc. (Freemont, Calif.) and Genpharm (San Jose, Calif.).

Completely human antibodies that recognize a selected epitope can be generated using a technique referred to as “guided selection”. In this approach a selected non-human monoclonal antibody, e.g., a mouse antibody, is used to guide the selection of a completely human antibody recognizing the same epitope. Human antibodies can also be produced using various techniques known in the art, including phage display libraries.

The antibody can be a fusion protein of an antibody, or a functionally active fragment thereof, and of the amino acid sequence of another protein (or portion thereof, such as at least 10, 20 or 50 amino acid portion of the protein) that is not the antibody, for example in which the antibody is fused via a covalent bond (e.g., a peptide bond), at either the N-terminus or the C-terminus of said amino acid sequence. For example, the antibody or fragment thereof may be covalently linked to the other protein at the N-terminus of the constant domain.

Antibodies include analogs and derivatives that are either modified, i.e., by the covalent attachment of any type of molecule as long as such covalent attachment permits the antibody to retain its antigen binding immunospecificity. For example, but not by way of limitation, the derivatives and analogs of the antibodies include those that have been further modified, e.g., by glycosylation, acetylation, pegylation, phosphorylation, amidation, derivatization by known protecting/blocking groups, proteolytic cleavage, linkage to a cellular antibody unit or other protein, etc. Any of numerous chemical modifications can be carried out by known techniques, including, but not limited to specific chemical cleavage, acetylation, formylation, metabolic synthesis in the presence of tunicamycin, etc. Additionally, the analog or derivative can contain one or more unnatural amino acids.

The antibodies conjugates can include antibodies having modifications (e.g., substitutions, deletions or additions) in amino acid residues that interact with Fc receptors. In particular, antibodies include antibodies having modifications in amino acid residues identified as involved in the interaction between the anti-Fc domain and the FcRn receptor. Antibodies immunospecific for a cancer cell antigen can be obtained commercially, for example, from Genentech (San Francisco, Calif.) or produced by any method known to one of ordinary skill in the art such as, e.g., chemical synthesis or recombinant expression techniques. The nucleotide sequence encoding antibodies immunospecific for a cancer cell antigen can be obtained, e.g., from the GenBank database or a database like it, the literature publications, or by routine cloning and sequencing.

In certain aspects, the antibody conjugates can be a monoclonal antibody, e.g. a murine monoclonal antibody, a chimeric antibody, or a humanized antibody. In some aspects, the antibody can be an antibody fragment, e.g. a Fab fragment.

Known antibodies for the treatment or prevention of cancer that can be conjugated are described herein. Antibodies immunospecific for a cancer cell antigen can be obtained commercially or produced by any method known to one of ordinary skill in the art such as, e.g., recombinant expression techniques. The nucleotide sequence encoding antibodies immunospecific for a cancer cell antigen can be obtained, e.g., from the GenBank database or a database like it, the literature publications, or by routine cloning and sequencing. Examples of antibodies available for the treatment of cancer include, but are not limited to, humanized anti-HER2 monoclonal antibody for the treatment of patients with metastatic breast cancer; RITUXAN® (rituximab; Genentech) which is a chimeric anti-CD20 monoclonal antibody for the treatment of patients with non-Hodgkin's lymphoma; OvaRex (oregovomab; AltaRex Corporation, MA) which is a murine antibody for the treatment of ovarian cancer; Panorex (edrecolomab, Glaxo Wellcome, NC) which is a murine IgG2a antibody for the treatment of colorectal cancer; Cetuximab Erbitux (cetuximab, Imclone Systems Inc., NY) which is an anti-EGFR IgG chimeric antibody for the treatment of epidermal growth factor positive cancers, such as head and neck cancer; Vitaxin (etaracizumab, MedImmune, Inc., MD) which is a humanized antibody for the treatment of sarcoma; Campath I/H (alemtuzumab, Leukosite, MA) which is a humanized IgG1 antibody for the treatment of chronic lymphocytic leukemia (CLL); Smart MI95 (Protein Design Labs, Inc., CA) which is a humanized anti-CD33 IgG antibody for the treatment of acute myeloid leukemia (AML); LymphoCide (epratuzumab, Immunomedics, Inc., NJ) which is a humanized anti-CD22 IgG antibody for the treatment of non-Hodgkin's lymphoma; Smart ID 10 (Protein Design Labs, Inc., CA) which is a humanized anti-HLA-DR antibody for the treatment of non-Hodgkin's lymphoma; Oncolym (Techniclone, Inc., CA) which is a radiolabeled murine anti-HLA-Dr1O antibody for the treatment of non-Hodgkin's lymphoma; Allomune (BioTransplant, CA) which is a humanized anti-CD2 mAb for the treatment of Hodgkin's Disease or non-Hodgkin's lymphoma; Avastin (bevacizumab, Genentech, Inc., CA) which is an anti-VEGF humanized antibody for the treatment of lung and colorectal cancers; Epratuzamab (Immunomedics, Inc., NJ and Amgen, CA) which is an anti-CD22 antibody for the treatment of non-Hodgkin's lymphoma; and CEAcide (Immunomedics, NJ) which is a humanized anti-CEA antibody for the treatment of colorectal cancer.

Other antibodies useful include, but are not limited to brentuximab (anti-CD30), polatuzumab (anti-CD79b), enfortumab (anti Nectin 4), tisotumad (anti tissue factor), loncastuximab (anti-CD19), rituximab (Anti-CD20), trastuzumab (anti HER2), inotuzumab (anti-CD22) gemtuzumab (anti-CD33), pertuzumab (anti-HER2), obinutuzumab (anti-CD20), ofatumumab (anti-CD20), olaratumab (anti-PDGFR-α), isatuximab (anti-CD38), Sacituzumab (anti-TROP2), U3-1784 (anti-FGFR4), daratumumab (anti-CD38), STI-6129 (anti-CD38), lintuzumab (anti-CD33), huMy9-6 (anti-CD33), belantamab (anti-BCMA), indatuximab (anti-CD138), cetuximab (anti-EGFR), dinutuximab (Anti-GD2), anti-CD38 A2 antibody, HuAT13/5 antibody, alemtuzumab (anti-CD52), ibritumomab (anti-CD20), tositumomab (anti-(anti-EGFR), CD20 conjugated to 1231), bevacizumab (anti-VEGF), panitumumab tremelimumab (formerly ticilimumab-anti-CTA-4), catumaxomab (anti-CD3 and EpCAM), oregovomab (anti CA125), h1959 antibody (anti-Gal-3BP), and veltuzumab (anti-CD20).

Other antibodies useful include, but are not limited to, antibodies against the following antigens: CA125 (ovarian), CA15-3 (carcinomas), CA19-9 (carcinomas), L₆ (carcinomas), Lewis Y (carcinomas), Lewis X (carcinomas), alpha fetoprotein (carcinomas), CA 242 (colorectal), placental alkaline phosphatase (carcinomas), prostate specific antigen (prostate), prostatic acid phosphatase (prostate), epidermal growth factor (carcinomas), MAGE-1 (carcinomas), MAGE-2 (carcinomas), MAGE-3 (carcinomas), MAGE-4 (carcinomas), anti-transferrin receptor (carcinomas), p97 (melanoma), MUC1-KLH (breast cancer), CEA (colorectal), gp1OO (melanoma), MARTI (melanoma), PSA (prostate), IL-2 receptor (T-cell leukemia and lymphomas), CD20 (non-Hodgkin's lymphoma), CD52 (leukemia), CD33 (leukemia), CD22 (lymphoma), human chorionic gonadotropin (carcinoma), CD38 (multiple myeloma), CD40 (lymphoma), mucin (carcinomas), P21 (carcinomas), MPG (melanoma), and Neu oncogene product (carcinomas). Some specific, useful antibodies include, but are not limited to, BR96 mAb (Trail, P. A., et al Science (1993) 261, 212-215), BR64 (Trail, P A, et al Cancer Research (1997) 57, 100-105), mAbs against the CD40 antigen, such as S2C6 mAb (Francisco, J. A., et al Cancer Res. (2000) 60:3225-3231), mAbs against the CD70 antigen, such as 1F6 mAb, and mAbs against the CD30 antigen, such as AC10. Many other internalizing antibodies that bind to tumor associated antigens can be used and have been reviewed.

Other antigens that the present conjugates can bind to include, but are not limited to, 5T4, ACE, ADRB3, AKAP-4, ALK, Androgen receptor, AOC3, APP, Axin1, AXL, B7H3, B7-H4, BCL2, BCMA, bcr-ab1, BORIS, BST2, C242, C4.4a, CA 125, CA6, CA9, CAIX, CCL11, CCR5, CD123, CD133, CD138, CD142, CD15, CD15-3, CD171, CD179a, CD18, CD19, CD19-9, CD2, CD20, CD22, CD23, CD24, CD25, CD27L. CD28, CD3, CD30, CD31, CD300LF, CD33, CD352, CD37, CD38, CD4, CD40, CD41, CD44, CD44v6, CD5, CD51, CD52, CD54, CD56, CD62E, CD62P, CD62L, CD70, CD71, CD72, CD74, CD79a, CD79b, CD80, CD90, CD97, CD125, CD138, CD141, CD147, CD152, CD154, CD326, CEA, CEACAM5, CFTR, clumping factor, cKit, Claudin 3, Claudin 18.2, CLDN6, CLEC12A, CLL-1, cll3, c-MET, Cripto protein, CS1, CTLA-4, CXCR2, CXORF61, Cyclin B1, CYP1B1, Cadherin-3, Cadherin-6. DLL3, E7, EDNRB, EFNA4, EGFR, EGFRVIII, ELF2M, EMR2, ENPP3, EPCAM, EphA2. Ephrin A4, Ephrin B2, EPHB4, ERBB2 (Her2/neu), ErbB3, ERG (TMPRSS2 ETS fusion gene), ETBR, ETV6-AML, FAP, FCAR, FCRL5, FGFR1, FGFR2, FGFR3, FGFR4, FLT3, Folate receptor alpha, Folate receptor beta, FOLR1, Fos-related antigen 1, Fucosyl Gal-3BP. GM1, GCC. GD2, GD3, GloboH, GM3, GPC1, GPC2, GPC3, gp1OO, GPNMB, GPR20, GPRC5D, GUCY2C, HAVCR1, HER2, HER3, HGF, HMF24, HMWMAA, HPV E6, hTERT, human telomerase reverse transcriptase, ICAM, ICOS-L, IFN-α, IFN-γ, IGF-I receptor, IGLL1, IL-2 receptor, IL-4 receptor, IL-13Ra2, IL-1 IRa, IL-1, IL-12, IL-23, IL-13, IL-22, IL-4, IL-5, IL-6, interferon receptor, integrins (including α4, αvβ3, αvβ5, αvβ6, α1β4, α4β1, α4β7, α5β1, α6β4, αIIbβ3 intergins), Integrin alphaV, intestinal carboxyl esterase, KIT, LAGE-1a, LAIR1, LAMP-1, LCK, Legumain, LewisY, LFA-1(CD11a), L-selectin (CD62L), LILRA2, LIV-1. LMP2, LRRC15, LY6E, LY6K. LY75, MAD-CT-1, MAD-CT-2, MAGE Al, Mel an A/M ARTI. Mesothelin, ML-IAP, MSLN, mucin, MUC1, MUC16, mut hsp70-2, MYCN, myostatin, NA17, NaPi2b, NCA-90, NCAM, Nectin-4, NGF, NOTCH1, NOTCH2, NOTCH3, NOTCH4, NY-BR-1, NY-ESO-1, o-acetyl-GD2, OR51E2. OY-TES1, p53, p53 mutant, PANX3, PAP, PAX3, PAX5, p-CAD, PCTA-1/Galectin 8, PD-L1, PD-L₂, PDGFR, PDGFR-beta, phosphatidylserine, PIK3CA, PLAC1, Polysialic acid, Prostase, prostatic carcinoma cell, prostein. Pseudomonas aeruginosa, rabies, survivin and telomerase, PRSS21, PSCA, PSMA, PTK7, RAGE-1, RANKL, Ras mutant, respiratory syncytial virus, Rhesus factor, RhoC, RON, ROR1, ROR2. RU1, RU2, sarcoma translocation breakpoints, SART3, SLAMF7, SLC44A4, sLe, SLITRK6, sperm protein 17, sphingosine-1-phosphate, SSEA-4, SSX2, STEAP1, TAG72, TARP. TCRb, TEM1/CD248, TEM7R, tenascin C. TF. TGF-1, TGF-b2, TNF-a, TGS5, Tie 2, TIM-1, Tn Ag. TRAC, TRAIL-R₁, TRAIL-R₂, TROP-2, TRP-2, TRPV1, TSHR, tumor antigen CTAA16.88, tyrosinase, UPK2, VEGF, VEGFR1, VEGFR2, vimentin, WT1, and/or XAGE1.

Antibodies that bind to antigens associated with antigen presenting cells such as CD40, OX40L, Endoglin, DEC-205, 4-1BBL, CD36, CD36, CD204, MARCO, DC-SIGN, CLEC9A, CLEC5A, Dectin 2, CLECIOA, CD206, CD64, CD32A, CDIA, HVEM, CD32B, PD-L1, BDCA-2, XCR-1, and CCR2 can also be conjugated.

Antibodies conjugate can bind to both a receptor or a receptor complex expressed on an activated lymphocyte. The receptor or receptor complex can comprise an immunoglobulin gene superfamily member, a TNF receptor superfamily member, an integrin, a cytokine receptor, a chemokine receptor, a major histocompatibility protein, a lectin, or a complement control protein. Non-limiting examples of suitable immunoglobulin superfamily members are CD2, CD3, CD4, CD8, CD19, CD22, CD28, CD79, CD90, CD 152/CTLA-4, PD-1, and ICOS. Non-limiting examples of suitable TNF receptor superfamily members are CD27, CD40, CD95/Fas, CD134/OX40, CD137/4-1BB, TNF-R1, TNFR-2, RANK, TACI, BCMA, osteoprotegerin, Apo2/TRAIL-R1, TRAIL-R2, TRAIL-R3, TRAIL-R4, and APO-3. Non-limiting examples of suitable integrins are CD11a, CD11b, CD11c, CD18, CD29, CD41, CD49a, CD49b, CD49c, CD49d, CD49c, CD49f, CD103, and CD104. Non-limiting examples of suitable lectins are C-type, S-type, and I-type lectin.

Preferably, the antibodies include 3F8, 8H9, abagovomab, abciximab (REOPRO®), abituzumab, abrezekimab, abrilumab, actoxumab, adalimumab (HUMIRA®), adecatumumab, aducanumab, afasevikumab, afelimomab, afutuzumab, alacizumab, ALD518, alemtuzumab (CAMPATH®), alirocumab (PRALUENT®), altumomab, amatuximab, anatumomab, andecaliximab, anctumab, anifrolumab, anrukinzumab, anti-TnC-A1-SIP (F16), apolizumab, aprutumab, arcitumomab (CEA-SCAN®), ascrinvacumab, asclizumab, atidortoxumab, atlizumab (tocilizumab, ACTEMRA®, ROACTEMRA®), atezolizumab (TECENTRIQ®), atinumab, atorolimumab, avelumab (Bavencio), azintuxizumab, balantamab, bapincuzumab, basiliximab (SIMULECT®), bavituximab, BCD-100, bectumomab (LYMPHOSCAN®), begelomab, belantamab, belimumab (BENLYSTAR), bemarituzumab, benralizumab (FASENRA®), bermekimab, bersanlimab, bertilimumab, besilesomab (SCINITIMUN®), bevacizumab (AVASTIN®), bezlotoxumab (ZINPLAVA®), biciromab (FIBRISCINT®), bimagrumab, bimekizumab, birtamimab, bivatuzumab, bleselumab, blinatumomab, blontuvetmab, blosozumab, bococizumab, brazikumab, brentuximab, briakinumab, brodalumab (SILIQ™), brolucizumab (BEOVU® brontictuzumab, burosumab (CRYSVITA®), cabiralizumab, caplacizumab (CABLIVI®), camidanlumab, camrelizumab, canakinumab (ILARIS®), cantuzumab, capromab, carlumab, carotuximab, catumaxomab (REMOVAB®), CBR96, CC49, cedelizumab, cemiplimab (LIBTAYO®), cergutuzumab, certrelimab, certolizumab, cetuximab (ERBITUX®), cibisatamab, cirmtuzumab, citatuzumab, cixutumumab, clazakizumab, clenoliximab, clivatuzumab, codrituzumab, cofctuzumab, coltuximab, conatumumab, concizumab, cosfroviximab, CR6261, crenezumab, crizanlizumab (ADAKVEO®), crotedumab, cusatuzumab, dacetuzumab, daclizumab (ZINBRYTA®), dalotuzumab, dapirolizumab, daratumumab (DARZALEX®), dectrekumab, demcizumab, denintuzumab, denosumab (PROLIA®), depatuxizumab, derlotuximab, detumomab, dezamizumab, dinutuximab (UNITUXIN®), diridavumab, domagrozumab, dostarlimab, dorlimomab, dorlixizumab, drozitumab, DS-8201, duligotuzumab, dupilumab (DUPIXENT®), durvalumab (IMFINZI®), dusigitumab, ccromeximab, eculizumab (SOLIRIS®), cdobacomab, cdrecolomab (PANOREX®), cfalizumab (RAPTIVA®), cfungumab (MYCOGRAB®), eldelumab, elezanumab, elgemtumab, elotuzumab (EMPLICITI®), clsilimomab, cmactuzumab cmapalumab (GAMIFANT®), emibctuzumab, emicizumab (HEMLIBRA®), cnapotamab, cnavatuzumab, enfortumab (PADCEV®), cnlimomab, cnoblituzumab, enokizumab, enoticumab, ensituximab, epitumomab, eptinezumab (VYEPTI®epratuzumab, erenumab (AIMOVIG®), crlizumab, ertumaxomab (REXOMUN″), ctaracizumab (ABEGRIN″), ctigilimab, ctrolizumab, evinacumab, evolocumab (REPATHA®), exbivirumab, fanolesomab (NEUTROSPEC®), faralimomab, faricimab, farletuzumab, fasinumab, FBTA05, felvizumab, fezakinumab, fibatuzumab, ficlatuzumab, figitumumab, firivumab, flanvotumab, fletikumab, flotetuzumab, fontolizumab (HUZAF®), foralumab, foravirumab, fremanczumab (AJOVY®), fresolimumab, frovocimab, fmnevetmab, fulranumab, futuximab, galcanezumab (EMGALITY®), galiximab, gancotamab, ganitumab, gantenerumab, gavilimomab, gedivumab, gemtuzumab, gevokizumab, gilvetmab, gimsilumab, girentuximab, glembatumumab, golimumab (SIMPONI®), gomiliximab, guselkumab (TREMFYA®), huMy9-6, OR000213, ianalumab, ibalizumab (TROGARZO®), IBI308, ibritumomab, icrucumab, idarucizumab (PRAXBFND®), ifabotuzumab, igovomab (INDIMACIS-125), iladatuzumab, IMAB362, imalumab, imaprelimab, imciromab (MYOSCINT®), imgatuzumab, inclacumab, indatuximab, indusatumab, inebilizumab, infliximab (REMICADE®), intetumumab, inolimomab, inotuzumab, iomab-B, ipilimumab, iratumumab, isatuximab (SARCLISA®), iscalimab, istiratumab, itolizumab, ixckizumab (TALTZ®), keliximab, labetuzumab (CEA-CIDE™), lacnotuzumab, ladiratuzumab, lampalizumab, lanadelumab (TAKHZYRO®), landogrozumab, laprituximab, larcaviximab, lebrikizumab, lemalesomab, lendalizumab, lenvervimab, lenzilumab, lerdelimumab, leronlimab, lesofavumab, letolizumab, lexatumumab, libivirumab, lifastuzumab, ligelizumab, lilotomab, lintuzumab, lirilumab, lodelcizumab, lokivetmab, loncastuximab, lorvotuzumab, losatuxizumab, lucatumumab, lulizumab, lumiliximab, lumretuzumab, lupartumab, lutikizumab, mapatumumab, margetuximab, marstacimab, maslimomab, matuzumab, mavrilimumab, mepolizumab (NUCALA®), metclimumab, milatuzumab, minretumomab, mirikizumab, mirvetuximab, mitumomab, modotuximab, molalizumab, mogamulizumab (POTELIGEO®), morolimumab, mosunetuzumab, motavizumab (NUMAX®), moxctumomab (LUMOXITI®), muromonab-CD3 (ORTHOCLONE OKT3®), nacolomab, namilumab, naptumomab, naratuximab, namatumab, natalizumab (TYSABRI®), navicixizumab, navivumab, naxitamab, nebacumab, necitumumab (PORTRAZZA®), nemolizumab, NEODOOI, nerelimomab, nesvacumab, netakimab, nimotuzumab (THERACIM®), nirsevimab, nivolumab, nofetumomab, obiltoxaximab (ANTHIM®), obinutuzumab, ocaratuzumab, ocrelizumab (OCREVUS®), odulimomab, ofatumumab (ARZERRA®), olaratumab (LARTRUVO®), olcclumab, olendalizumab, olokizumab, omalizumab (XOLAIR®), omburtamab, OMS721, onartuzumab, ontecizumab, ontuxizumab, onvatilimab, opicinumab, oportuzumab, oregovomab (OVAREX), orticumab, otelixizumab, otilimab, otlertuzumab, oxelumab, ozanezumab, ozogamicin, ozoralizumab, pagibaximab, palivizumab (SYNAGIS®), pamrevlumab, panitumumab (VECTIBIX®), pankomab, panobacumab, parsatuzumab, pascolizumab, pasotuxizumab, pateclizumab, patritumab, PDR001, pembrolizumab, pemtumomab (THERAGYN®), perakizumab, pertuzumab (OMNITARG®), pexelizumab, pidilizumab, pinatuzumab, pintumomab, placulumab, polatuzumab (Polivy), prezalumab, plozalizumab, pogalizumab, ponezumab, porgaviximab, prasinezumab, prezalizumab, priliximab, pritoxaximab, pritumumab, PRO 140, quilizumab, racotumomab, radretumab, rafivirumab, ralpancizumab, ramucirumab, ranevetmab, ranibizumab (LUCENTIS®), ravagalimab, ravulizumab (ULTOMIRIS®), raxibacumab, refanczumab, regavirumab, REGN-EB3, renatlimab, remtolumab, reslizumab (CINQAIR®), rilotumumab, rinucumab, risankizumab (SKYRIZI®), rituximab (RITUXAN®), rivabazumab, rmab, robatumumab, roledumab, romilkimab, romosozumab (EVENITY®), rontalizumab, rosmantuzumab, rovalpituzumab, rovelizumab (LEUKARREST®), rozanolixizumab, ruplizumab (ANTOVA), SA237, sacituzumab, samalizumab, samrotamab, sarilumab (KEVZARA®), satralizumab, satumomab pendetide, secukinumab (COSENTYX®), selicrelumab, scribantumab, setoxaximab, setmsumab, sevimmab, SGN-CD19A, SHP647, sibrotuzumab, sifalimumab, siltuximab, simtuzumab, siplizumab, sirtratumab, sirukumab, sofituzumab, solanczumab, solitomab, sonepcizumab, sontuzumab, spartalizumab, stamulumab, STI-6129, sulesomab (LEUKOSCAN®), suptavumab, sutimlimab, suvizumab, suvratoxumab, tabalumab, tacatuzumab (AFP-CIDE®), tadocizumab, talacotuzumab, talizumab, tamtuvetmab, tanczumab, taplitumomab paptox, tarextumab, tavolimab, tefibazumab (AUREXIS®), telimomab, telisotuzumab, tesidolumab, tetraxctan, tetulomab, tenatumomab, tencliximab, teprotumumab (TEPEZZA®), teplizumab, tezepelumab, TGN1412, tibulizumab, ticilimumab (TREMELIMUMAB®), tigatuzumab, timigutuzumab, timolumab, tiragolumab, tiragotumab, tislelizumab, tisotumab, tiuxctan, tildrakizumab (ILUMYA®), TNX-650, tocilizumab (atlizumab, ACTEMRA®), tomuzotuximab, toralizumab, tosatoxumab, tositumomab (BEXXAR®), tovetumab, tralokinumab, trastuzumab (HERCEPTIN®), TRBS07, tregalizumab, tremelimumab, trevogrumab, tucotuzumab, tuvirumab, urtoxazumab, ustekinumab (STELERA®), ublituximab, ulocuplumab, urelumab, utomilumab, vadastuximab, vanalimab, vandortuzumab, vantictumab, vanucizumab, vapaliximab, varisacumab, varlilumab, vatelizumab, vedolizumab, veltuzumab, vepalimomab, vesencumab, visilizumab (NUVION®), vobarilizumab, volociximab (HUMASPECT®), vonlerolizumab, vopratelimab, vorsetuzumab, votumumab, vunakizumab, xentuzumab, XMAB-5574, zalutumumab (HuMEX-EGFr), zanolimumab (HuMAX-CD4), zatuximab, zenocutuzumab, ziralimumab, zolbetuximab or zolimomab.

An antibody “which binds” a target of interest, such as a molecular target or an antigen of interest, is one capable of binding that antigen with sufficient affinity such that the antibody is useful in targeting a cell expressing the antigen.

In a further particular embodiment, P is a peptide, preferably having specific targeting capabilities of protein receptors which are overexpressed in tumour tissues. These peptides often have precedent in the literature for having a strong binding affinity to the target site within the nanomole magnitude measured by different techniques including for example surface plasmon resonance (SPR), biolayer interferometry (BLI) and isothermal titration calorimetry (ITC). More particularly, the peptide is a tripeptide RGD (arg-gly-asp) targeting integrins (α5β1, α8β1 and αIIbβ3) or GnRH (gonadotropin-releasing hormone) targeting GnRH-R (receptor version of the hormone) or SST (somatostatin) targeting SSTR1-5 (somatostatin receptor) or EGF (epidermal growth factor) targeting EGFR: HER1, HER2, HER3, HER4 or Angiopep-2 targeting LRP-1 (low-density lipoprotein receptor-related protein-1) or Bradykinin-potentiating peptides (BPPs) targeting ACE (Angiotensin-converting enzyme receptor).

The conjugates of formula (II) as above defined can also be represented by the following formula III:

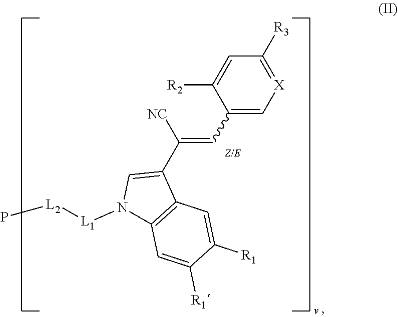

In this aspect, X, R₁, R_{1′}, R₂, and R₃, are such as defined in the above “payload” section including all the particular and preferred embodiments. P and v are such as above defined.

In this aspect, L₃ represents a linker allowing to bound the peptide or the protein P to the “payload” (molecule or indole derivative). As used herein, the term “linker” refers to any chemical moiety capable of connecting the peptide or the protein “P” to the “molecule”.

In an embodiment, the linker L₃ can contain a heterobifunctional group. As used herein, the term “heterobifunctional group” refers to a chemical moiety that connects the linker of which it is a part to the peptide or the protein P. Heterobifunctional groups are characterized as having different reactive groups at either end of the chemical moiety. Attachment to “P” can be accomplished through chemical or enzymatic conjugation, or a combination of both. Chemical conjugation involves the controlled reaction of accessible amino acid residues on the surface of the protein P with a reaction handle on the heterobifunctional group. Examples of chemical conjugation include, but are not limited to, lysine amide coupling, cysteine coupling, and coupling via a non-natural amino acid incorporated by genetic engineering, wherein non-natural amino acid residues with a desired reaction handle are installed onto “P”. In enzymatic conjugation, an enzyme mediates the coupling of the linker with an accessible amino residue on the peptide or protein P. Examples of enzymatic conjugation include, but are not limited to, transpeptidation using sortase, transpeptidation using microbial transglutaminase, and N-glycan engineering. Chemical conjugation and enzymatic conjugation may also be used sequentially. For example, enzymatic conjugation can also be used for installing unique reaction handles on “P” to be utilized in subsequent chemical conjugation. Particularly, the heterobifunctional group is selected from:

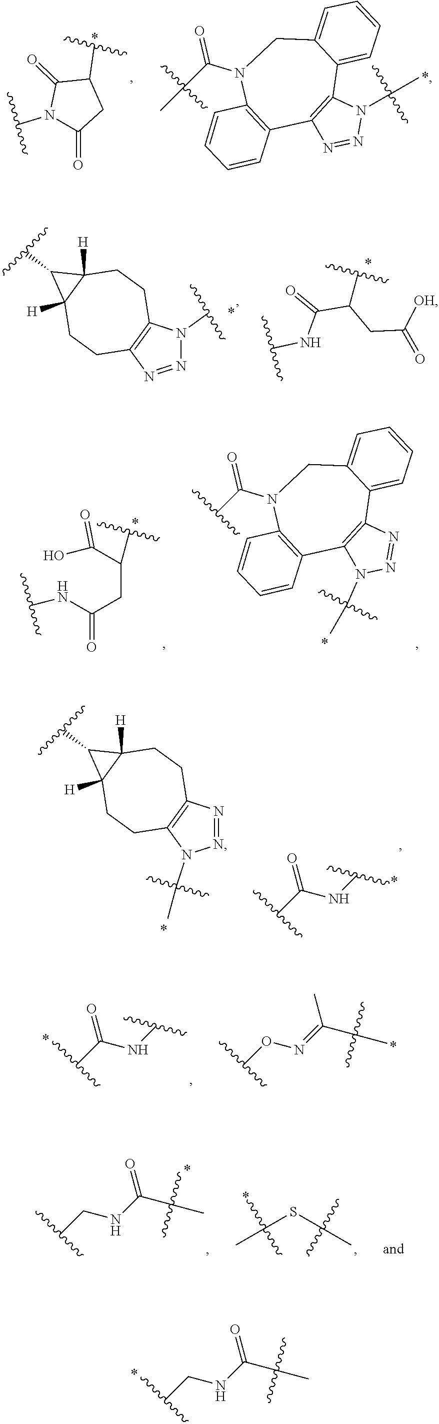

wherein [symbol] is the point of attachment to the remaining portion of the linker L₃, and [symbol] the point of attachment to “P”.

In an embodiment, the linker L₃ is an enzymatic cleavable group by a protease such as cathepsin B, VGAP tetrapeptide and the like. Particularly, the linker L₃ is a protease cleavable linker selected from:

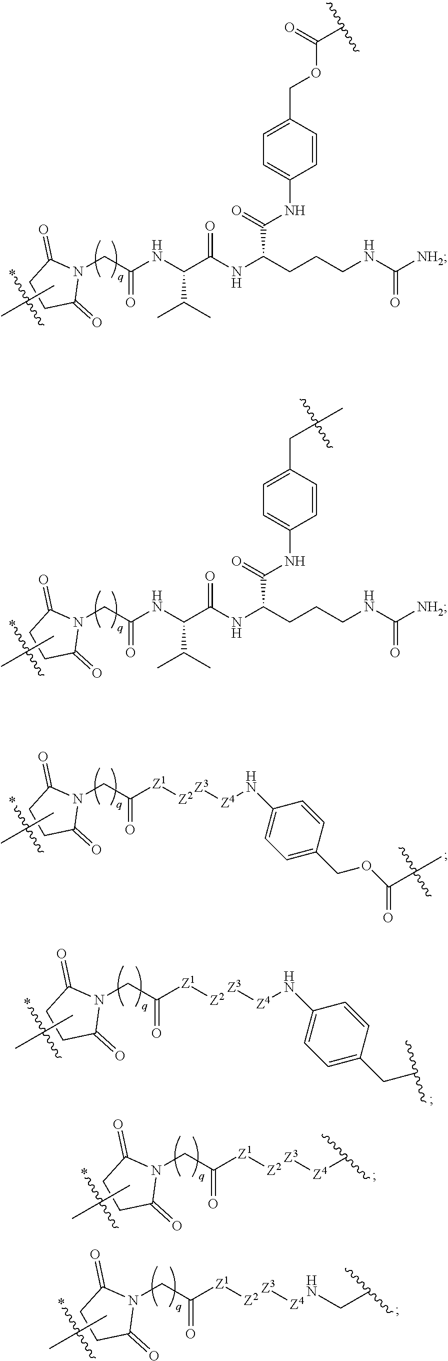

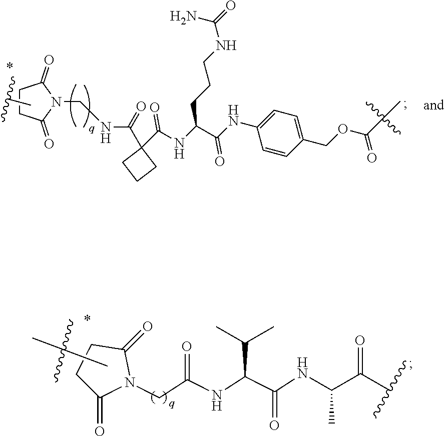

in which q is an integer from 2 to 10; Z¹, Z², Z³, and Z⁴ are each independently absent or a naturally-occurring amino acid residue in the L- or D-configuration, provided that at least two of Z¹, Z², Z³, and Z⁴ are amino acid residues; [symbol] is the point of attachment to the molecule; and [symbol] is the point of attachment to “P”. Preferably, Z¹, Z², Z³, and Z⁴ are independently absent or selected from the group consisting of L-valine, D-valine, L-citrulline, D-citrulline, L-alanine, D-alanine, L-glutamine, D-glutamine, L-glutamic acid, D-glutamic acid, L-aspartic acid, D-aspartic acid, L-asparagine, D-asparagine, L-phenylalanine, D-phenylalanine, L-lysine, D-Lysine, and glycine; provided that at least two of Z¹, Z², Z³, and Z⁴ are amino acid residues. More preferably, Z¹ is absent or glycine; Z² is absent or selected from L-glutamine, D-glutamine, L-glutamic acid, D-glutamic acid, L-aspartic acid, D-aspartic acid, L-alanine, D-alanine, and glycine; Z³ is selected from L-valine, D-valine, L-alanine, D-alanine, L-phenylalanine, D-phenylalanine, and glycine; and Z⁴ is selected from L-alanine, D-alanine, L-citrulline, D-citrulline, L-asparagine, D-asparagine, L-lysine, D-lysine, L-phenylalamine, D-phenylalanine, and glycine. Even more preferably, L₃ is

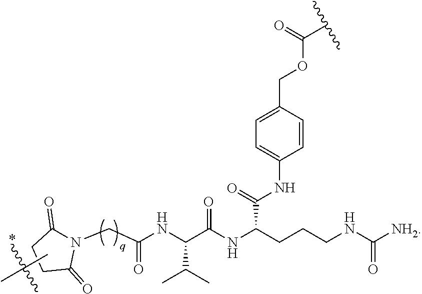

with q is 5, and [symbol] is the point of attachment to the molecule; and [symbol] is the point of attachment to “P”.

In an embodiment, the linker L₃ is a pyrophosphatase cleavable linker, preferably a pyrophosphatase cleavable linker having the following formula:

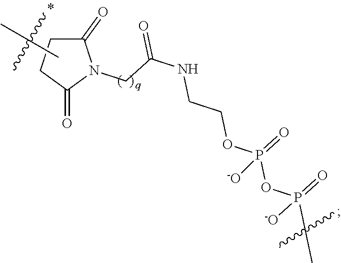

with q is an integer from 2 to 10 and [symbol] is the point of attachment to the molecule; and [symbol] is the point of attachment to “P”.

In an embodiment, the linker L₃ is a beta-glucuronidase cleavable linker, preferably selected from

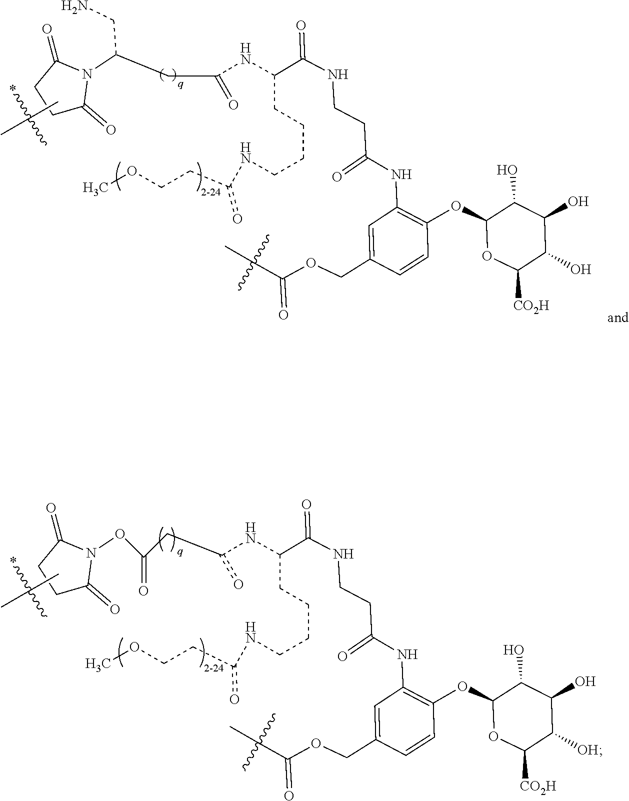

with q is an integer from 2 to 10; - - - is absent or a bond; and [symbol] is the point of attachment to the molecule; and [symbol] is the point of attachment to “P”.

In an embodiment, the linker L₃ is bioreductible. Bioreductible linkers take advantage of the difference in reduction potential in the intracellular compartment versus plasma. Reduced glutathione presented in tumor cells' cytoplasm is up to 1000-fold higher than that present in normal cells' cytoplasma, and the tumor cells also contain enzymes which can contribute to reduction in cellular compartments. The linkers keep conjugates intact during systemic circulation, and are selectively cleaved by the high intracellular concentration of glutathione, releasing the active drugs at the tumor sites from the non-toxic prodrugs. Preferably L₃ is a bioreductible linker selected from:

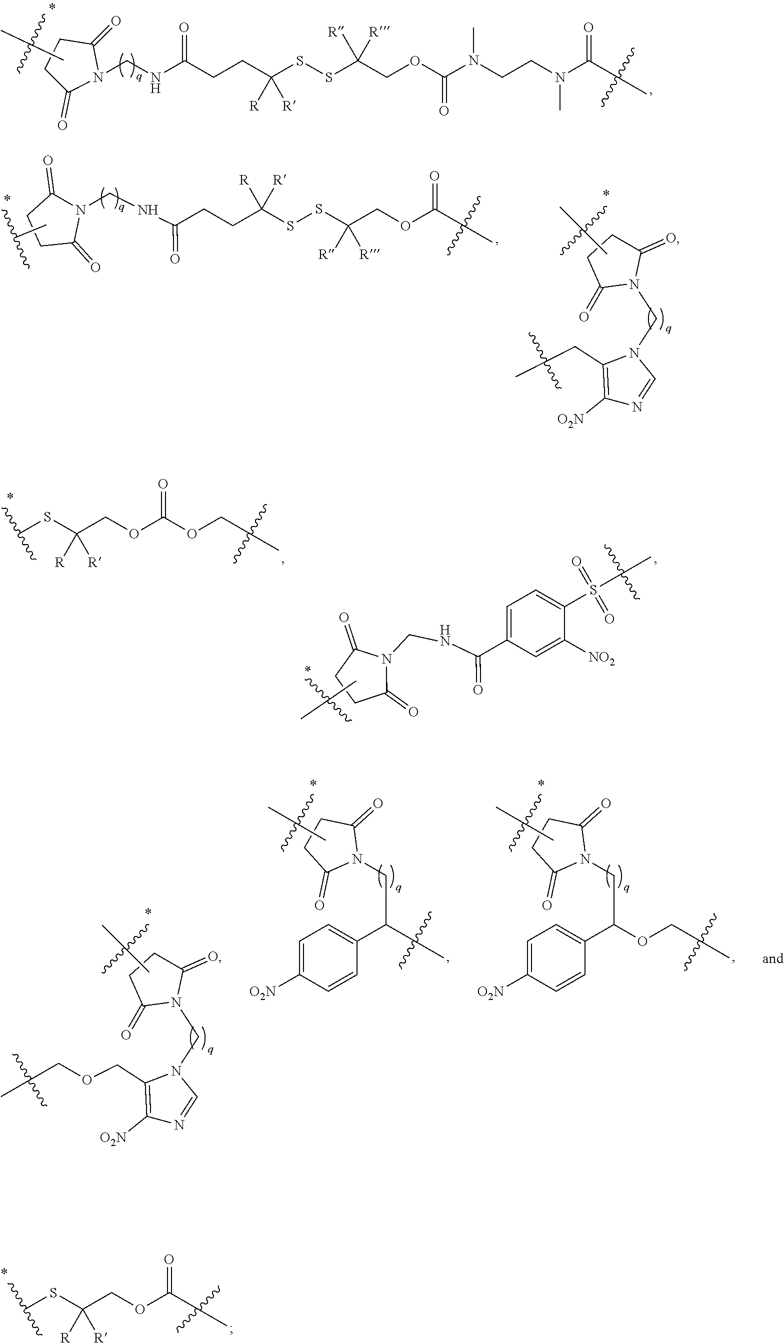

with q is an integer from 2 to 10; R, R′, R″, and R′″ are each independently selected from hydrogen, C₁-C₆alkoxyC₁-C₆alkyl, (C₁-C₆)₂NC₁-C₆alkyl, and C₁-C₆alkyl, or, two geminal R groups, together with the carbon atom to which they are attached, can form a cyclobutyl or cyclopropyl ring; and [symbol] is the point of attachment to the molecule; and [symbol] is the point of attachment to “P”.

In an embodiment, the linker L₃ is acid cleavable. Preferably, L₃ is an acid cleavable liker selected from

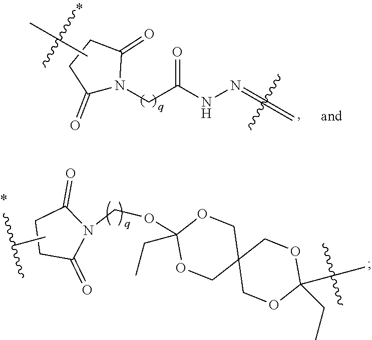

with q is an integer from 2 to 10; and [symbol] is the point of attachment to the molecule; and [symbol] is the point of attachment to “P”.

In an embodiment, L₃ is a click-to-release linker, where release of the molecule is chemically triggered by a tetrazine or related compound. Preferably, L₃ is a click-to-release linker selected from:

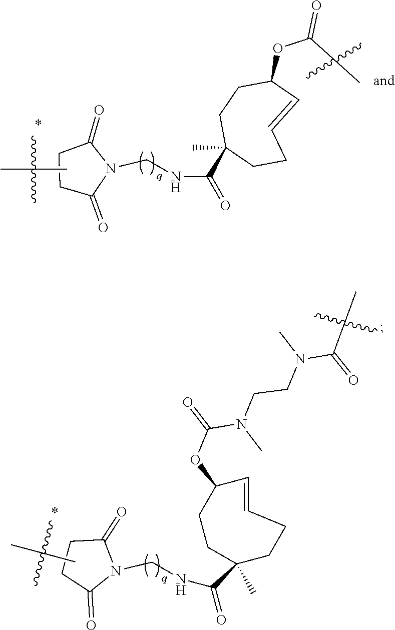

with q is an integer from 2 to 10; and [symbol] is the point of attachment to the molecule; and [symbol] is the point of attachment to “P”.

In an embodiment, L₃ is a linker L₁-L₂ with L₁ is a cleavable group defined in the above “L₁ series” section including all the particular and preferred embodiments; and L₂ is a binding protein connector defined in the above “L₁ series” section including all the particular and preferred embodiments.

The present invention further provides “payload” or “drugs” as above defined and compounds of formula (I) as defined herein conjugated with a nanoparticle, at particular a silica nanoparticle. In a particular aspect, the “payload” can be substituted with a linker to form a linker-payload conjugate which is attached to the silica nanoparticle, forming thereby a nanoparticle-drug conjugate. Examples of such nanoparticles-drug conjugates (NDC) are for instance described in the international patent applications WO 2022/093800 and WO 2022/093794.

A preferred aspect is a nanoparticle-drug conjugate comprising:
(a) a nanoparticle that comprises a silica-based core and a silica shell surrounding at least a portion of the core; polyethylene glycol (PEG) covalently bonded to the surface of the nanoparticle, and optionally a fluorescent compound covalently encapsulated within the core of the nanoparticle;
(b) a targeting ligand that binds to a folate receptor, wherein the targeting ligand is selected from the group consisting of folic acid, dihydrofolic acid, tetrahydrofolic acid, and folate receptor binding derivatives of any of the foregoing, and wherein the targeting ligand is attached to the nanoparticle directly or indirectly through a spacer group; and
(c) a compound of formula (I) as defined herein whit R^{a} is a group of formula (B)

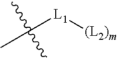

in which L₁ is a cleavable group chosen a pH-sensitive group, a photo-induced cleavable group, a bioreductible cleavable group, and an enzymatic cleavable group, and m is equal to 0; wherein said compound of formula (I) is attached to the nanoparticle directly or indirectly through a spacer group.

### Therapeutic Uses

The present invention relates to:
a pharmaceutical composition comprising any compound having the formula (I), (II), (III) as defined above including anyone of the disclosed embodiments; and/or
a pharmaceutical composition comprising any compound having the formula (I), (II), or (III) as defined above including anyone of the disclosed embodiments, and a pharmaceutically acceptable excipient; and/or
a pharmaceutical composition comprising (a) any compound having the formula (I), (II), or (III) as defined above or including anyone of the disclosed embodiments, and (b) an additional active ingredient, preferably an additional antitumoral drug; and/or
a pharmaceutical composition as defined above or any compound having the formula (I), (II) or (III) as defined above including anyone of the disclosed embodiments for use as a drug; and/or
a pharmaceutical composition as defined above or any compound having the formula (I), (II) or (III) as defined above, for use in the treatment of cancer; and/or
a product or kit containing (a) any compound of formula (I), (II) or (III) as disclosed above including anyone of the disclosed embodiments and (b) an additional active ingredient, preferably an additional antitumoral drug, as a combined preparation for simultaneous, separate or sequential use, in particular in the treatment of cancer; and/or
a combined preparation which comprises (a) any compound of formula (I), (II) or (III) as disclosed above including anyone of the disclosed embodiments and (b) an additional active ingredient, preferably an additional antitumoral drug, for simultaneous, separate or sequential use, in particular in the treatment of cancer; and/or
a pharmaceutical composition as defined above or any compound having the formula (I), (II) or (III) as defined above including anyone of the disclosed embodiments, for the use in the treatment of cancer in combination with radiotherapy, surgery (e.g., tumor resection), hyperthermia and/or other antitumoral therapies or before, simultaneously or after surgery (e.g., tumor resection); and/or
the use of a pharmaceutical composition as defined above or any compound having the formula (I), (II) or (III) as defined above including anyone of the disclosed embodiments, for the manufacture of a medicament for the treatment of cancer; and/or
the use of a pharmaceutical composition as defined above or any compound having the formula (I), (II) or (III) as defined including anyone of the disclosed embodiments and (b) an additional active ingredient, preferably an additional antitumoral drug, for the manufacture of a medicament for the treatment of cancer; and/or
a method for treating a cancer in a subject in need thereof, comprising administering an effective amount of a pharmaceutical composition as defined above or any compound having the formula (I), (II), or (III) as defined above including anyone of the disclosed embodiments; and/or
a method for treating a cancer in a subject in need thereof, comprising administering an effective amount of a pharmaceutical composition as defined above or any compound having the formula (I), (II), or (III) including anyone of the disclosed embodiments, and (b) an additional active ingredient, preferably an additional antitumoral drug; and/or
a method for treating a cancer in a subject in need thereof, comprising administering an effective amount of a pharmaceutical composition as defined above or any compound having the formula (I), (II), or (III) as defined above including anyone of the disclosed embodiments, in combination with radiotherapy, surgery (e.g., tumor resection), hyperthermia and/or other antitumoral therapies.

The term “cancer”, as used herein, refers to the presence of cells possessing characteristics typical of cancer-causing cells, such as uncontrolled proliferation, immortality, metastatic potential, rapid growth and proliferation rate, and certain characteristic morphological features. The cancer may be solid tumor or hematopoietic tumor. Examples of cancer include, for example, leukemia, lymphoma, blastoma, carcinoma and sarcoma. More particular examples of such cancers include chronic myeloid leukemia, acute lymphoblastic leukemia, Philadelphia chromosome positive acute lymphoblastic leukemia (Ph+ ALL), squamous cell carcinoma, lung cancer, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, osteosarcoma, pancreatic cancer, glioblastoma multiforme, cervical cancer, stomach cancer, bladder cancer, hepatoma, breast cancer, oesophagal cancer, colon carcinoma, and head and neck cancer, gastric cancer, germ cell tumor, pediatric sarcoma, sinonasal natural killer, multiple myeloma, acute myelogenous leukemia (AML), chronic lymphocytic leukemia, mastocytosis and any symptom associated with mastocytosis. Preferably, the cancer is leukemia, acute myelogenous leukemia, lymphoma, breast cancer, pancreatic cancer, lung cancer or colon cancer. More preferably, the cancer is a acute myelogenous leukemia or a pancreatic cancer. Optionally, the cancer is associated with a dysregulation of MKlp2 or its pathway. In particular, the cancer is associated with an overexpression of MKlp2.

As used herein, the term “treatment”, “treat” or “treating” refers to any act intended to ameliorate the health status of patients such as therapy, prevention, prophylaxis and retardation of the disease. In certain embodiments, such term refers to the amelioration or eradication of a disease or symptoms associated with a disease. In other embodiments, this term refers to minimizing the spread or worsening of the disease resulting from the administration of one or more therapeutic agents to a subject with such a disease.

By “effective amount” it is meant the quantity of the pharmaceutical composition of the invention which prevents, removes or reduces the deleterious effects of the treated disease in mammals, including humans. It is understood that the administered dose may be adapted by those skilled in the art according to the patient, the pathology, the mode of administration, etc. For instance, the compounds of the invention may be used at a dose of 0.01 to 500 mg/kg of body weight/day. In a particular embodiment, the pharmaceutical composition according to the invention comprises 0.01 to 500 mg/kg of the compound of the invention. It is understood that the administered dose may be adapted by those skilled in the art according to the patient, the pathology, the mode of administration, etc.

The administration route can be topical, transdermal, oral, rectal, sublingual, intranasal, intrathecal, intratumoral or parenteral (including subcutaneous, intramuscular, intravenous and/or intradermal). Preferably, the administration route is parental, oral or topical. The pharmaceutical composition is adapted for one or several of the above-mentioned routes. The pharmaceutical composition, kit, product or combined preparation is preferably administered by injection or by intravenous infusion or suitable sterile solutions, or in the form of liquid or solid doses via the alimentary canal.

The pharmaceutical composition can be formulated as solutions in pharmaceutically compatible solvents or as emulsions, suspensions or dispersions in suitable pharmaceutical solvents or vehicles, or as pills, tablets or capsules that contain solid vehicles in a way known in the art. Formulations of the present invention suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. Formulations for rectal administration may be in the form of a suppository incorporating the active ingredient and carrier such as cocoa butter, or in the form of an enema. Formulations suitable for parenteral administration conveniently comprise a sterile oily or aqueous preparation of the active ingredient which is preferably isotonic with the blood of the recipient. Every such formulation can also contain other pharmaceutically compatible and nontoxic auxiliary agents, such as, e.g. stabilizers, antioxidants, binders, dyes, emulsifiers or flavouring substances. The formulations of the present invention comprise an active ingredient in association with a pharmaceutically acceptable carrier therefore and optionally other therapeutic ingredients. The carrier must be “acceptable” in the sense of being compatible with the other ingredients of the formulations and not deleterious to the recipient thereof. The pharmaceutical compositions are advantageously applied by injection or intravenous infusion of suitable sterile solutions or as oral dosage by the digestive tract. Methods for the safe and effective administration of most of these chemotherapeutic agents are known to those skilled in the art. In addition, their administration is described in the standard literature.

The additional antitumoral drug can be selected in the non-exhaustive list of antitumoral agents consisting of an inhibitor of topoisomerases I or II, an anti-mitotic agent, a DNA alkylating agent, an anti-metabolic agent, a targeted agent such as a kinase inhibitor, a therapeutical antibody and/or an antibody-drug conjugates designed to mediate cytotoxicity against the cancer cells or to modulate one of their key biological functions.

Anti-mitotic agents include, but are not limited to, paclitaxel, docetaxel and analogs such as larotaxel (also called XRP9881; Sanofi-Aventis), XRP6258 (Sanofi-Aventis), BMS-184476 (Bristol-Meyer-Squibb), BMS-188797 (Bristol-Meyer-Squibb), BMS-275183 (Bristol-Meyer-Squibb), ortataxel (also called IDN 5109, BAY 59-8862 or SB-T-101131; Bristol-Meyer-Squibb), RPR 109881A (Bristol-Meyer-Squibb), RPR 116258 (Bristol-Meyer-Squibb), NBT-287 (TAPESTRY), PG-paclitaxel (also called CT-2103, PPX, paclitaxel poliglumex, paclitaxel polyglutamate or Xyotax™), ABRAXANE® (also called Nab-Paclitaxel; ABRAXIS BIOSCIENCE), Tesetaxel (also called DJ-927), IDN 5390 (INDENA), Taxoprexin (also called docosahexanoic acid-paclitaxel; PROTARGA), DHA-paclitaxel (also called Taxoprexin®), and MAC-321 (WYETH).

Inhibitors of topoisomerases I and/or II include, but are not limited to, etoposide, topotecan, camptothecin, irinotecan, amsacrine, intoplicin, anthracyclines such as doxorubicin, epirubicin, daunorubicin, idarubicin and mitoxantrone. Inhibitors of Topoisomerase I and II include, but are not limited to, intoplicin.

DNA alkylating agent includes, but are not limited to, cisplatin, carboplatin and oxaliplatin. In a preferred embodiment, the DNA alkylating agent is cisplatin.

Anti-metabolic agents block the enzymes responsible for nucleic acid synthesis or become incorporated into DNA, which produces an incorrect genetic code and leads to apoptosis. Non-exhaustive examples thereof include, without limitation, folic acid antagonists, pyrimidine analogs, purine analogs and adenosine deaminase inhibitors, and more particularly Methotrexate, Floxuridine, Cytarabine, 6-Mercaptopurine, 6-Thioguanine, Fludarabine phosphate, Pentostatine, 5-fluorouracil, gemcitabine and capecitabine.

The anti-tumoral agent can be alkylating agents including, without limitation, nitrogen mustards, ethylenimine derivatives, alkyl sulfonates, nitrosoureas, metal salts and triazenes. Non-exhaustive examples thereof include Uracil mustard, Chlormethine, Cyclophosphamide (CYTOXAN(R)), Ifosfamide, Melphalan, Chlorambucil, Pipobroman, Triethylenemelamine, Triethylenethiophosphoramine, Busulfan, Carmustine, Lomustine, cisplatin, carboplatin, oxaliplatin, thiotepa, Streptozocin, Dacarbazine, and Temozolomide.

The anti-tumoral agent can also be a targeted agent, in particular a kinase inhibitor. The kinase may be selected from the group consisting of intracellular tyrosine or serine/threonine kinases, receptors tyrosine or serine/threonine kinase. For instance, the agents may have ability to inhibit angiogenesis based on the inhibitory activities on VEGFR and PDGFR kinases. In particular, the targeted agent can be selected among the multiple kinase inhibitor drugs which are already approved: Gleevec, which inhibits Abl, and Iressa and Tarceva, which both inhibit EGFR, Sorafenib (Nexavar, BAY 43-9006) which inhibits Raf, Dasatinib (BMS-354825) and Nilotinib (AMN-107, Tasigna) which also inhibits Abl, Lapatinib which also inhibits EGFR, Temsirolimus (Torisel, CCI-779) which targets the mTOR pathway, Sunitinib (Stuten, SU11248) which inhibits several targets including VEGFR as well as specific antibodies inactivating kinase receptors: Herceptin and Avastin.

Antibody-drug conjugates include, but are not limited to, gemtuzumab ozogamicin, brentuximab vedotin, trastuzumab emtansine, inotuzumab ozogamicin, polatuzumab vedotin, enfortumab vedotin, trastuzumab deruxtecan (Enhertu®), sacituzumab govitecan (Trodelvy®), belantamab mafodotin (Blenrep®), trastuzumab duocarmazine, BAT8001, mirvetuximab soravtansine, SAR408701, loncastuximab tesirine, camidanlumab tesirine, vadastuximab talirine, rovalpituzumab tesirine or depatuxizumab mafodotin

The term “therapy”, as used herein, refers to any type of treatment of cancer (i.e., antitumoral therapy), including an adjuvant therapy and a neoadjuvant therapy. Therapy comprises radiotherapy and therapies, preferably systemic therapies such as hormone therapy, chemotherapy, immunotherapy and monoclonal antibody therapy.

The term “adjuvant therapy”, as used herein, refers to any type of treatment of cancer given as additional treatment, usually after surgical resection of the primary tumor, in a patient affected with a cancer that is at risk of metastasizing and/or likely to recur. The aim of such an adjuvant treatment is to improve the prognosis. Adjuvant therapies comprise radiotherapy and therapy, preferably systemic therapy, such as hormone therapy, chemotherapy, immunotherapy and monoclonal antibody therapy.

The term “hormone therapy” or “hormonal therapy” refers to a cancer treatment having for purpose to block, add or remove hormones. For instance, in breast cancer, the female hormones estrogen and progesterone can promote the growth of some breast cancer cells. So, in these patients, hormone therapy is given to block estrogen and a non-exhaustive list commonly used drugs includes: Tamoxifen, Toremifene, Anastrozole, Exemestane, Letrozole, Goserelin/Leuprolide, Megestrol acetate, and Fluoxymesterone.

As used herein, the term “chemotherapeutic treatment” or “chemotherapy” refers to a cancer therapeutic treatment using chemical or biological substances, in particular using one or several antineoplastic agents.

The term “radiotherapeutic treatment” or “radiotherapy” is a term commonly used in the art to refer to multiple types of radiation therapy including internal and external radiation therapies or radioimmunotherapy, and the use of various types of radiations including X-rays, gamma rays, alpha particles, beta particles, photons, electrons, neutrons, radioisotopes, and other forms of ionizing radiations.

Further aspects and advantages of the present invention will be described in the following examples, which should be regarded as illustrative and not limiting.

### EXAMPLES

### I. “Phosphonic Prodrugs”

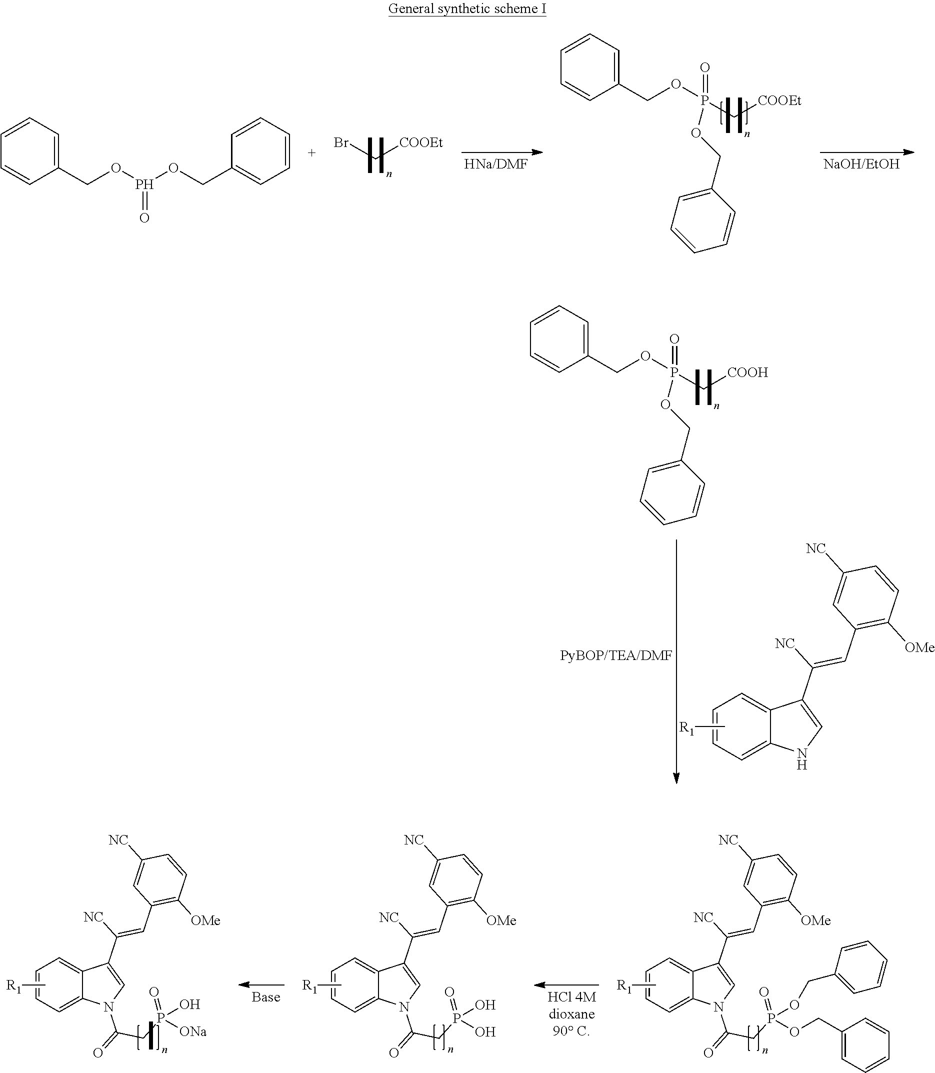

Condensation of the dibenzyl phosphite with bromalkanoates gives the phosphono-esters, which provide the phophono-acids after basic hydrolysis. Then the acids are coupled to the NH-indole of the drug via a peptide coupling agent to give the benzyl-phosphonate prodrugs, which are hydrolyzed to the prodrugs phosphonic acid in an acidic media. Treatment with a base affords the phosphonic prodrugs as salts.

### Procedure for the Preparation of the Phosphonic Prodrugs

Phosphonoalkanoic acids were synthesized according to C. Zinglé et al. Bioorganic & Medicinal Chemistry letters, 22(21), 6563-6567 (2012).

### A. Phosphonoalkanoate Esters (Step 1)

### 1. Ethyl 5-(bis(benzyloxy)phosphoryl)pentanoate

To a Suspension of Sodium Hydride (1.141 g, 28.5 Mmol) in DMF (40 mL) Wad Added Slowly dibenzyl phosphite (7 mL, 28.5 mmol) in DMF (20 mL) at 0° C. under argon atmosphere. The reaction mixture was stirred for 15 min at room temperature, cooled again to 0° C. and ethyl 5-bromovalerate (4.51 mL, 28.5 mmol) was added. The reaction mixture was stirred at RT for 1 hr, poured into 150 mL of water and extracted with 150 mL of EtOAc. The organic layer was washed twice with water (75 mL). The combined aqueous layers were extracted again with 75 mL of EtOAc. The combined organic layers were washed with brine, dried over Na₂SO₄, filtered and concentrated under reduced pressure to give ethyl 5-(bis(benzyloxy)phosphoryl)pentanoate as a colorless oil (11.41 g). Yield-87%

LC-MS: [M+H]=391

¹H NMR (300 MHz, DMSO) δ ppm: 7.45-7.20 (m, 10H), 5.10-4.86 (m, 4H), 4.02 (q, J=7.3 Hz, 2H), 2.29 (dt, J=14.3, 7.4 Hz, 2H), 1.81 (ddd, J=10.7, 7.9, 5.9 Hz, 2H), 1.70-1.35 (m, 4H), 1.15 (t, J=7.1 Hz, 3H)

The following compounds were prepared using the same procedure as above:

### 2. Ethyl 3-(bis(benzyloxy)phosphoryl)propanoate

Yield=25%

LC-MS: [M+H]=363

1H NMR (300 MHz, DMSO) δ ppm: 7.37 (s, 10H), 4.99 (qd, J=12.1, 8.1 Hz, 4H), 4.03 (q, J=7.1 Hz, 2H), 2.37 (t, J=7.3 Hz, 2H), 2.25-2.05 (m, 2H), 1.15 (t, J=7.1 Hz, 3H)

### 3. Ethyl 7-(bis(benzyloxy)phosphoryl)heptanoate

Yield=99%

LC-MS: [M+H]=419

1H NMR (300 MHz, DMSO) δ ppm: 7.43-7.24 (m, 10H), 4.97 (qd, J=12.1, 8.3 Hz, 4H), 4.03 (q, J=7.1, 2H), 2.24 (dt, J=12.2, 7.3 Hz, 2H), 1.88-1.67 (m, 2H), 1.57-1.31 (m, 6H), 1.34-1.18 (m, 2H), 1.16 (t, J=7.1, 3H)

### B. Phosphonoalkanoate acids (step 2)

### 1. 5-(bis(benzyloxy)phosphoryl)pentanoic acid

To a solution of ethyl 5-(bis(benzyloxy)phosphoryl)pentanoate (11.41 g. 29.2 mmol) in ethanol (100 mL) was added 1N sodium hydroxide 1N (58.5 mL, 58.5 mmol). The reaction mixture was stirred at RT overnight. The reaction mixture was diluted with 200 mL of water and washed with ethyl acetate (3×100 mL). The combined organic layers were washed with water. The aqueous layers were acidified to pH=3-4 with 1N HCl (60 mL) and then extracted three times with ethyl acetate (100 mL). The combined organic layers were washed with brine, dried over Na₂SO₄, filtered and concentrated to dryness to give 5-(bis(benzyloxy)phosphoryl)pentanoic acid as a colorless oil (7.93 g). Yield=74%

LC-MS: [M+H]=363

¹H NMR (300 MHz, DMSO) δ ppm: 7.46-7.23 (m, 10H), 5.10-4.85 (m, 4H), 2.18 (t. J=7.0 Hz, 2H), 1.88-1.71 (m, 2H), 1.61-1.38 (m, 4H)

Using the same procedure, the following compounds were prepared:

### 2. 3-(bis(benzyloxy)phosphoryl)propanoic acid

Yield=87%

LC-MS: [M+H]=335

¹H NMR (300 MHz, DMSO) δ ppm: 12.35 (s, 1H), 7.43-7.27 (m, 10H), 5.11-4.88 (m, 4H), 2.45-2.34 (m, 2H), 2.13-1.97 (m, 2H)

### 3. 7-(bis(benzyloxy)phosphoryl)heptanoic acid

Yield=83%

LC-MS: [M+H]=391

¹H NMR (300 MHz, DMSO) δ ppm: 7.45-7.24 (m, 10H), 4.96 (qd, J=12.1, 8.2 Hz, 4H), 2.17 (dt, J=10.8, 7.3 Hz, 2H), 1.86-1.67 (m, 2H), 1.53-1.33 (m, 4H), 1.23 (ddt, J=32.4, 13.4, 6.6 Hz, 4H)

### C. Prodrug Moiety Coupling (Step 3)

### 1. (Z)-dibenzyl-5-(5-bromo-3-(1-cyano-2-(5-cyano-2-methoxyphenyl)vinyl)-1H-indol-1-yl)-5-oxopentylphosphonate

To a solution of (Z)-3-(2-(5-bromo-1H-indol-3-yl)-2-cyanovinyl)-4-methoxybenzonitrile (2.10 g. 5.55 mmol), 5-(bis(benzyloxy)phosphoryl)pentanoic acid (2.40 g, 6.62 mmol) and PyBOP (4.31 g. 8.28 mmol) in DMF (30 mL) was added TEA (1.539 mL, 11.04 mmol). The reaction mixture was stirred at RT overnight and then poured into 200 mL of water. The resulting suspension was stirred at RT for 30 min, filtered, washed twice with water (50 mL) and dried under vacuum to give (Z)-dibenzyl 5-(5-bromo-3-(1-cyano-2-(5-cyano-2-methoxyphenyl)vinyl)-1H-indol-1-yl)-5-oxopentylphosphonate (4.10 g) as a pale yellow solid.

Yield=51%

LC-MS: [M+H]=722

¹H NMR (300 MHz, DMSO) δ ppm: 8.39 (d, J=8.5 Hz, 2H), 8.29 (d, J=1.7 Hz, 1H), 8.16 (d, J=1.8 Hz, 1H), 8.01 (dd, J=8.7, 2.1 Hz, 1H), 7.94 (s, 1H), 7.62 (dd, J=8.9, 1.9 Hz, 1H), 7.40-7.30 (m, 11H), 5.08-4.90 (m, 4H), 3.97 (s, 3H), 3.15 (t, J=7.1 Hz, 2H), 2.01-1.85 (m, 2H), 1.75 (dd, J=14.2, 7.0 Hz, 2H), 1.62 (m, 2H)

The following compounds were prepared using the same procedure:

### 2. (Z)-dibenzyl-3-(5-bromo-3-(1-cyano-2-(5-cyano-2-methoxyphenyl)vinyl)-1H-indol-1-yl)-3-oxopropylphosphonate

Yield=74%

LC-MS: [M+H]=694

1H NMR (300 MHz, DMSO) δ ppm: 8.35 (d, J=7.6 Hz, 2H), 8.30 (d, J=1.8 Hz, 1H), 8.15 (d, J=1.8 Hz, 1H), 8.01 (dd, J=8.7, 2.1 Hz, 1H), 7.97 (s, 1H), 7.62 (dd, J=8.9, 1.9 Hz, 1H), 7.43-7.23 (m, 11H), 5.14-4.94 (m, 4H), 3.98 (s, 3H), 3.43-3.35 (m, 2H), 2.29 (dt, J=8.3, 7.3 Hz, 2H)

### 3. (Z)-dibenzyl-7-(5-bromo-3-(1-cyano-2-(5-cyano-2-methoxyphenyl)vinyl)-1H-indol-1-yl)-7-oxoheptylphosphonate

Yield=52%

LC-MS: [M+H]=750

1H NMR (300 MHz, CDCl3) δ ppm: 8.38 (d, J=8.9 Hz, 1H), 8.21 (d, J=1.8 Hz, 1H), 7.93 (d. J=1.8 Hz, 1H), 7.79 (s, 1H), 7.72 (s, 1H), 7.66 (dd, J=8.7, 2.0 Hz, 1H), 7.47 (dd, J=8.9, 1.9 Hz, 1H), 7.26 (s, 10H), 6.97 (d, J=8.7 Hz, 1H), 4.93 (ddd, J=30.1, 11.8, 8.5 Hz, 4H), 3.93 (s, 3H), 2.83 (t, J=7.3 Hz, 2H), 1.86-1.34 (m, 10H)

### 4. (Z)-dibenzyl-5-(3-(1-cyano-2-(5-cyano-2-methoxyphenyl)vinyl)-6-methoxy-1H-indol-1-yl)-5-oxopentylphosphonate

Yield=52%

LC-MS: [M+H]=674

¹H NMR (300 MHz, DMSO) δ ppm: 8.28 (d, J=1.8 Hz, 1H), 8.15 (s, 1H), 8.05 (d, J=2.4 Hz, 1H), 8.02-7.97 (m, 1H), 7.93 (s, 1H), 7.88 (d, J=8.8 Hz, 1H), 7.44-7.25 (m, 11H), 7.06 (dd, J=8.8, 2.4 Hz, 1H), 4.99 (p, J=12.1 Hz, 4H), 3.97 (s, 3H), 3.83 (s, 3H), 3.13 (t, J=6.9 Hz, 2H), 2.02-1.85 (m, 2H), 1.75 (dd, J=13.9, 7.0 Hz, 2H), 1.69-1.53 (m, 2H)

### 5. (Z)-dibenzyl-7-(3-(1-cyano-2-(5-cyano-2-methoxyphenyl)vinyl)-6-methoxy-1H-indol-1-yl)-7-oxoheptylphosphonate

Yield=46%

LC-MS: [M+H]=702

1H NMR (300 MHz, DMSO) δ ppm: 8.27 (d, J=1.9 Hz, 1H), 8.14 (s, 1H), 8.06 (d, J=2.4 Hz, 1H), 7.99 (dd, J=8.7, 2.1 Hz, 1H), 7.94 (s, 1H), 7.88 (d, J=8.8 Hz, 1H), 7.40-7.30 (m, 11H), 7.06 (dd, J=8.8, 2.4 Hz, 1H), 4.98 (qd, J=12.1, 8.2 Hz, 4H), 3.98 (d, J=5.1 Hz, 3H), 3.09 (t, J=7.1 Hz, 2H), 1.88-1.75 (m, 2H), 1.71-1.59 (m, 2H), 1.47 (d, J=7.7 Hz, 2H), 1.41-1.32 (m, 4H)

### D. Phosphonic Acid Prodrugs (Step 4)

### Example 1: (Z)-5-(5-bromo-3-(1-cyano-2-(5-cyano-2-methoxyphenyl)vinyl)-1H-indol-1-yl)-5-oxopentylphosphonic acid

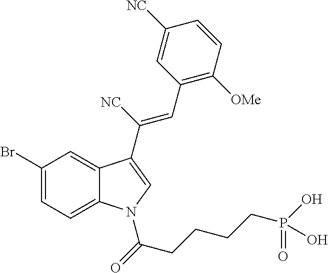 [SMILES: COc1ccc(C#N)cc1/C=C(\C#N)c1cn(C(=O)CCCCP(=O)(O)O)c2ccc(Br)cc12]

(Z)-dibenzyl-5-(5-bromo-3-(1-cyano-2-(5-cyano-2-methoxyphenyl)vinyl)-1H-indol-1-yl)-5-oxopentylphosphonate (1.531 g, 2.119 mmol) was dissolved in 4M HCl in 1,4-dioxane (47.7 ml, 190.8 mmol) to give a pale yellow solution. The reaction mixture was heated at 90° C. for 2 h. After cooling, the precipitate was filtered, washed twice with dioxane (3 ml) and diisopropylether (20 ml) successively to give (Z)-5-(5-bromo-3-(1-cyano-2-(5-cyano-2-methoxyphenyl)vinyl)-1H-indol-1-yl)-5-oxopentylphosphonic acid as a light grey solid (1.003 g). Yield-86%

LC-MS: [M+H]=541.9

¹H NMR (300 MHz, DMSO) δ ppm: 8.48-8.35 (m, 2H), 8.30 (d, J=1.9 Hz, 1H), 8.16 (d, J=1.8 Hz, 1H), 8.02 (dd, J=8.7, 2.1 Hz, 1H), 7.96 (s, 1H), 7.62 (dd, J=8.9, 1.9 Hz, 1H), 7.38 (d, J=8.8 Hz, 1H), 3.99 (s, 3H), 3.18 (t, J=7.1 Hz, 2H), 1.69 (dd, J=49.3, 8.9 Hz, 6H)

Using the same procedure, the following compounds were prepared:

### Example 2: (Z)-3-(5-bromo-3-(1-cyano-2-(5-cyano-2-methoxyphenyl)vinyl)-1H-indol-1-yl)-3-oxopropylphosphonic acid

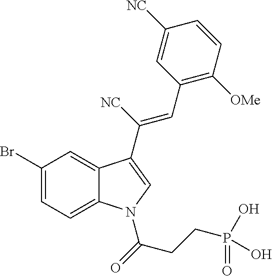 [SMILES: COc1ccc(C#N)cc1/C=C(\C#N)c1cn(C(=O)CCP(=O)(O)O)c2ccc(Br)cc12]

Yield=96%

LC-MS: [M+H]=513.9

¹H NMR (300 MHz, DMSO) δ ppm: 8.42-8.31 (m, 2H), 8.28 (d, J=1.7 Hz, 1H), 8.17 (d, J=1.8 Hz, 1H), 8.04-7.94 (m, 2H), 7.63 (dd, J=8.9, 1.9 Hz, 1H), 7.37 (d, J=8.8 Hz, 1H), 3.99 (s, 3H), 3.33 (d, J=6.8 Hz, 2H), 2.04-1.90 (m, 2H)

### Example 3: (Z)-7-(5-bromo-3-(1-cyano-2-(5-cyano-2-methoxyphenyl)vinyl)-1H-indol-1-yl)-7-oxoheptylphosphonic acid

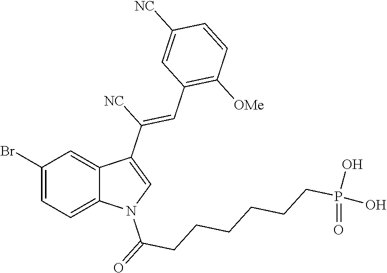 [SMILES: COc1ccc(C#N)cc1/C=C(\C#N)c1cn(C(=O)CCCCCCP(=O)(O)O)c2ccc(Br)cc12]

Yield=98%

LC-MS: [M+H]=570

¹H NMR (300 MHz, DMSO) δ ppm: 8.40 (d, J=7.7 Hz, 2H), 8.28 (s, 1H), 8.14 (s, 1H), 8.00 (d, J=8.6 Hz, 1H), 7.94 (s, 1H), 7.60 (d, J=8.9 Hz, 1H), 7.37 (d, J=8.8 Hz, 1H), 3.97 (s, 3H), 3.15 (t, J=7.1 Hz, 2H), 1.69 (s, 2H), 1.44 (d, J=25.9 Hz, 8H)

### Example 4: (Z)-5-(3-(1-cyano-2-(5-cyano-2-methoxyphenyl)vinyl)-6-methoxy-1H-indol-1-yl)-5-oxopentylphosphonic acid

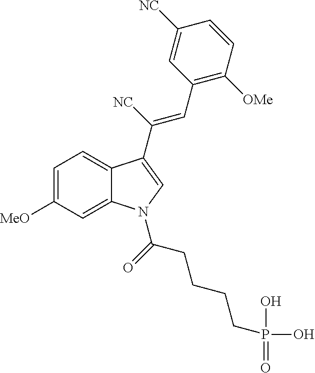 [SMILES: COc1ccc2c(/C(C#N)=C/c3cc(C#N)ccc3OC)cn(C(=O)CCCCP(=O)(O)O)c2c1]

Yield=69%

LC-MS: [M+H]=494

¹H NMR (300 MHz, DMSO) δ ppm: 8.25 (s, 1H), 8.16 (s, 1H), 8.04 (s, 1H), 7.98 (d, 1H), 7.91 (s, 1H), 7.86 (d, 1H), 7.35 (d, 1H), 7.03 (dd, 1H), 3.97 (s, 3H), 3.83 (s, 3H), 3.15 (t, J=7 Hz, 2H), 1.77 (t, J=7 Hz, 2H), 1.61 (m, 4H)

### Example 5: (Z)-7-(3-(1-cyano-2-(5-cyano-2-methoxyphenyl)vinyl)-6-methoxy-1H-indol-1-yl)-7-oxoheptylphosphonic acid

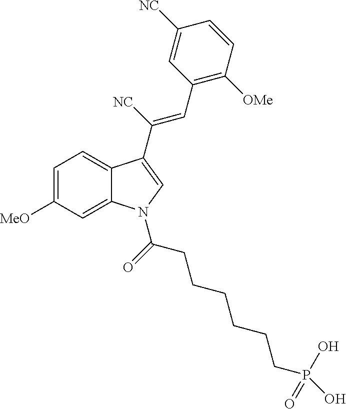 [SMILES: COc1ccc2c(/C(C#N)=C/c3cc(C#N)ccc3OC)cn(C(=O)CCCCCCP(=O)(O)O)c2c1]

Yield=73%

LC-MS: [M+H]=522

### E. Phosphonic Acid Sodium Salt Prodrugs (Step 5)

### Example 6: Sodium (Z)-hydrogen5-(5-bromo-3-(1-cyano-2-(5-cyano-2-methoxyphenyl)vinyl)-1H-indol-1-yl)-5-oxopentylphosphonate

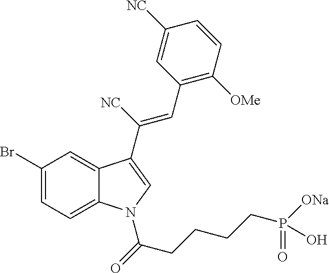

(Z)-5-(5-bromo-3-(1-cyano-2-(5-cyano-2-methoxyphenyl)vinyl)-1H-indol-1-yl)-5-oxopentylphosphonic acid (700 mg, 1.291 mmol) was suspended in water (25 mL) followed by addition at +5° C. of 1N sodium hydroxide (1.291 mL, 1.291 mmol). The reaction mixture was stirred at RT for 0.5 h to obtain a clear solution. The solution was lyophilized to give sodium (Z)-hydrogen5-(5-bromo-3-(1-cyano-2-(5-cyano-2-methoxyphenyl)vinyl)-1H-indol-1-yl)-5-oxopentylphosphonate as a pale yellow solid (0.72 g)

Yield=98%

LC-MS: [M+H]=562

¹H NMR (300 MHz, DMSO) δ ppm: 12.12 (brs, 1H), 8.5-7.0 (m, 8H), 3.96 (brs, 3H), 3.53 (m, 6H), 1.69 (m, 2H)

Using the same protocol, the following compounds were obtained:

### Example 7: sodium (Z)-hydrogen3-(5-bromo-3-(1-cyano-2-(5-cyano-2-methoxyphenyl)vinyl)-1H-indol-1-yl)-3-oxopropylphosphonate

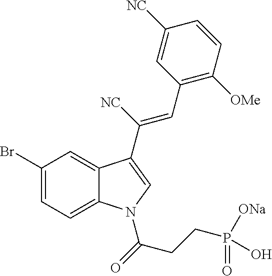

Yield=97%

LC-MS: [M−H]=511.9

¹H NMR (300 MHz, DMSO) δ ppm: 12.04 (brs, 1H), 8.21 (s, 1H), 8.08 (s, 1H), 7.90 (m, 2H), 7.70 (s, 1H), 7.48 (d, 1H), 7.35 (m, 2H), 3.97 (s, 3H), 3.41 (brs, 4H)

### Example 8: sodium (Z)-hydrogen4-(5-bromo-3-(1-cyano-2-(5-cyano-2-methoxyphenyl)vinyl)-1H-indol-1-yl)-4 oxobutylphosphonate

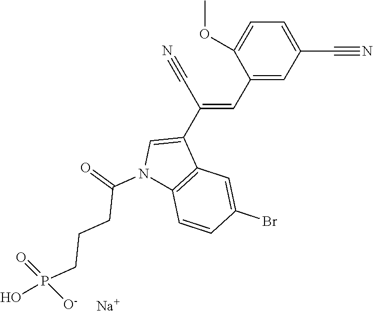 [SMILES: COc1ccc(C#N)cc1/C=C(\C#N)c1cn(C(=O)CCCP(=O)([O-])O)c2ccc(Br)cc12 ; [Na+]]

LC-MS: [M+H]=528

### Example 9: sodium (Z)-hydrogen6-(5-bromo-3-(1-cyano-2-(5-cyano-2-methoxyphenyl)vinyl)-1H-indol-1-yl)-4-oxohexylphosphonate

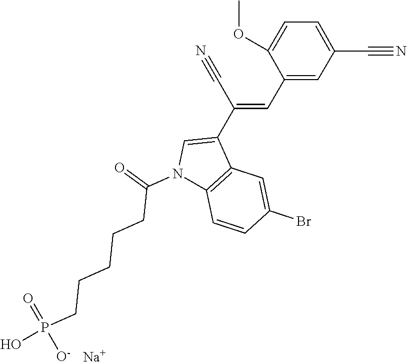 [SMILES: COc1ccc(C#N)cc1/C=C(\C#N)c1cn(C(=O)CCCCCP(=O)([O-])O)c2ccc(Br)cc12 ; [Na+]]

LC-MS: [M+H]=556

### Example 10: sodium (Z)-hydrogen7-(5-bromo-3-(1-cyano-2-(5-cyano-2-methoxyphenyl)vinyl)-1H-indol-1-yl)-7-oxoheptylphosphonate

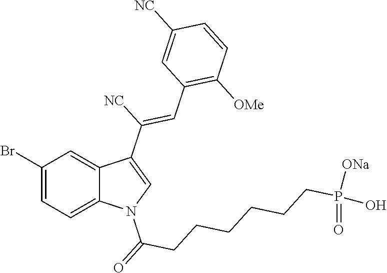

Yield=98%

LC-MS: [M−H]=568

¹H NMR (300 MHz, DMSO) δ ppm: 12.11 (brs, 1H), 8.25-7.35 (m, 8H), 3.97 (s, 3H), 3.59 (brs, 8H), 1.37 (brs, 4H)

### Example 11: sodium (Z)-hydrogen5-(3-(1-cyano-2-(5-cyano-2-methoxyphenyl)vinyl)-6-methoxy-1H-indol-1-yl)-5-oxopentylphosphonate

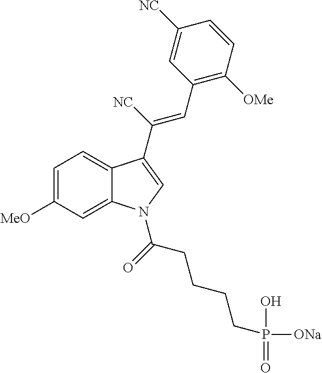

### Yield Quantitative

LC-MS: [M+H]=494

¹H NMR (300 MHz, D₂O) δ ppm: 7.5-6.5 (m, 8H), 3.59 (s, 6H), 3.25 (brs, 2H), 1.57 (m, 6H)

### Example 12: sodium (Z)-hydrogen7-(3-(1-cyano-2-(5-cyano-2-methoxyphenyl)vinyl)-6-methoxy-1H-indol-1-yl)-7-oxoheptylphosphonate

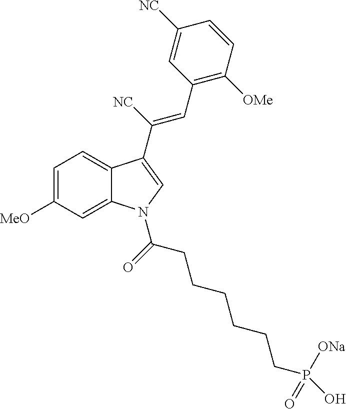

Yield=96%

LC-MS: [M+H]=522

### Example 13: disodium (Z)-4-(5-bromo-3-(1-cyano-2-(5-cyano-2-methoxyphenyl)vinyl)-1H-indol-1-yl)-4-oxobutylphosphonate

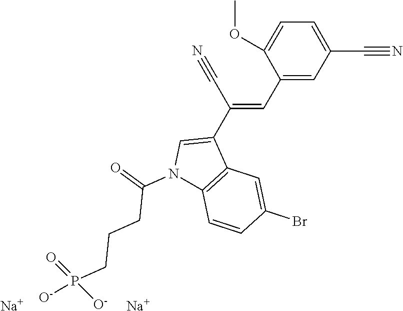 [SMILES: COc1ccc(C#N)cc1/C=C(\C#N)c1cn(C(=O)CCCP(=O)([O-])[O-])c2ccc(Br)cc12 ; [Na+] ; [Na+]]

(Z)-4-(5-bromo-3-(1-cyano-2-(5-cyano-2-methoxyphenyl)vinyl)-1H-indol-1-yl)-4-oxobutylphosphonic acid (74.2 mg, 0.14 mmol) and sodium bicarbonate (23.6 mg, 0.281 mmol) were suspended in water (10 mL) and heated at 40° C. for 10 min to give a clear solution. The solution was lyophilized to give (Z)-4-(5-bromo-3-(1-cyano-2-(5-cyano-2-methoxyphenyl)vinyl)-1H-indol-1-yl)-4-oxobutylphosphonic acid disodium salt as a pale yellow solid (0.72 g).

LC-MS: [M+H]=528

¹H NMR (500 MHZ, D₂O) δ ppm: 7.9-6.7 (m, 10H), 3.78 (s, 3H), 2.9 (brs, 2H), 1.9 (d, 2H), 1.5 (d, 2H)

### Example 14: disodium (Z)-5-(5-bromo-3-(1-cyano-2-(5-cyano-2-methoxyphenyl)vinyl)-1H-indol-1-yl)-5-oxopentylphosphonate

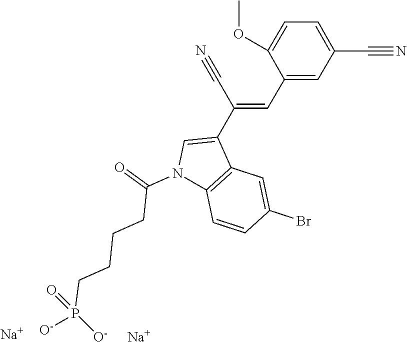 [SMILES: [Na+] ; COc1ccc(C#N)cc1/C=C(\C#N)c1cn(C(=O)CCCCP(=O)([O-])[O-])c2ccc(Br)cc12 ; [Na+]]

(Z)-5-(5-bromo-3-(1-cyano-2-(5-cyano-2-methoxyphenyl)vinyl)-1H-indol-1-yl)-5-oxopentylphosphonic acid (229 mg, 0.422 mmol) was suspended in water (10 mL) followed by addition of 1N sodium hydroxide (0.845 mL, 0.845 mmol). Acetone (50 mL) was slowly added, the precipitate was filtered and washed twice with acetone (5 mL) to give (Z)-5-(5-bromo-3-(1-cyano-2-(5-cyano-2-methoxyphenyl)vinyl)-1H-indol-1-yl)-5-oxopentylphosphonate disodium salt as a pale yellow solid (0.214 g).

LC-MS: [M+H]=542

### F. Phosphonic Acid Lysine Salt Prodrugs (Step 5)

### Example 15: (S)-2,6-diaminohexanoic acid compound with (Z)-3-(5-bromo-3-(1-cyano-2-(5-cyano-2-methoxyphenyl)vinyl)-1H-indol-1-yl)-3-oxopropylphosphonic acid (2:1)

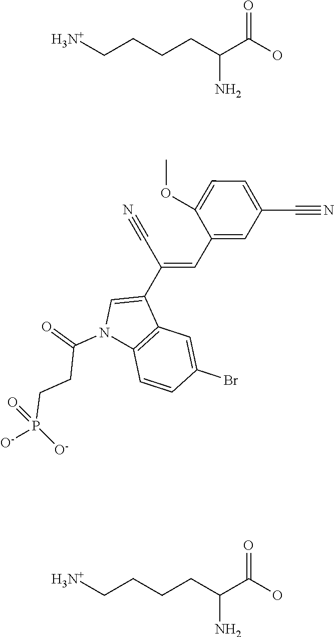 [SMILES: NC(CCCC[NH3+])C(=O)O ; COc1ccc(C#N)cc1/C=C(\C#N)c1cn(C(=O)CCP(=O)([O-])[O-])c2ccc(Br)cc12 ; NC(CCCC[NH3+])C(=O)O]

(Z)-hydrogen3-(5-bromo-3-(1-cyano-2-(5-cyano-2-methoxyphenyl)vinyl)-1H-indol-1-yl)-3-oxopropylphosphonic acid (0.399 g, 0.776 mmol) was suspended in water (10 mL). L-Lysine (0.227 g, 1.552 mmol) was added and the reaction mixture was stirred at RT for 10 min until complete solubilization. Then acetone (50 mL) was slowly added, the precipitate filtered, washed with acetone (2×5 mL) and dried to give a yellow solid (0.52 g). Yield=78%

LC-MS: [M−H]=511.9

### Example 16: (S)-2,6-diaminohexanoic acid compound with (Z)-4-(5-bromo-3-(1-cyano-2-(5-cyano-2-methoxyphenyl)vinyl)-1H-indol-1-yl)-4-oxobutylphosphonic acid (2:1)

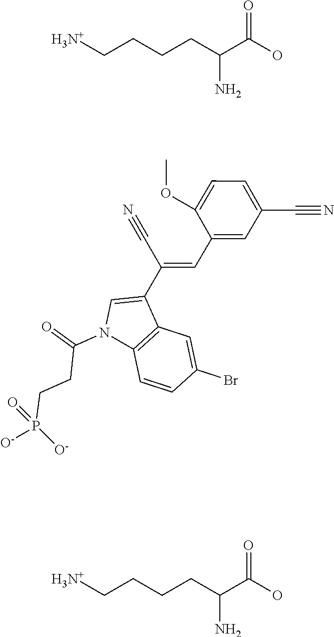 [SMILES: NC(CCCC[NH3+])C(=O)O ; COc1ccc(C#N)cc1/C=C(\C#N)c1cn(C(=O)CCP(=O)([O-])[O-])c2ccc(Br)cc12 ; NC(CCCC[NH3+])C(=O)O]

LC-MS: [M−H]=525.8

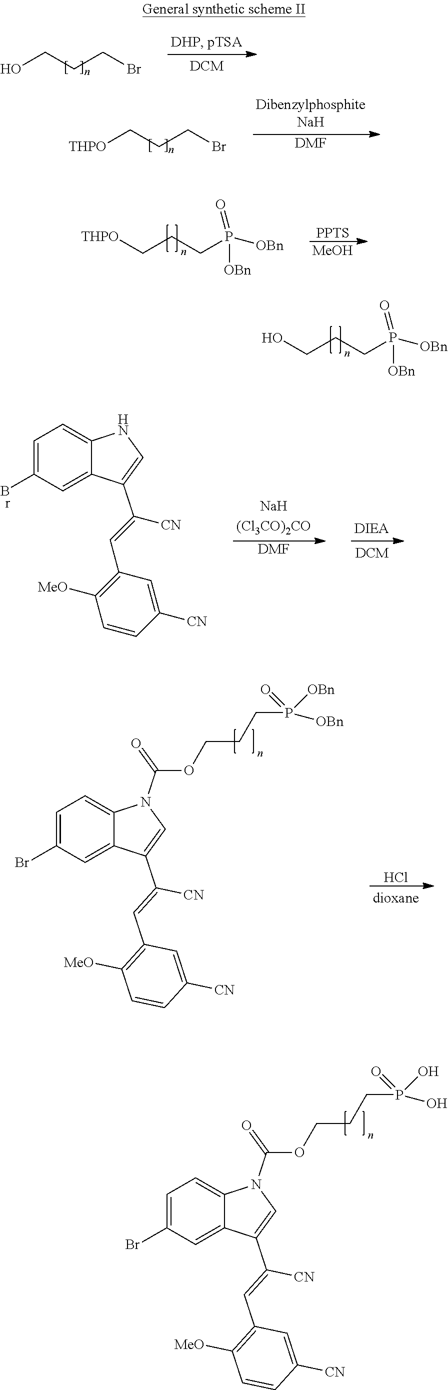

Condensation of the dibenzyl phosphite with THP-protected bromoalkanols give the phosphono-THP-protected alcohols, which provide the phophono-alcohol after acid hydrolysis. Then the NH-indole of the drug is treated with triphosgene to give the corresponding chloroformate, which reacts with the phosphono-alcohols to give the benzyl-phosphonate prodrugs. These latter compounds are hydrolyzed to the prodrugs phosphonic acid in an acidic media.

### Example 17:3-[hydroxy(dioxo)-lambda6-phosphanyl]propyl 5-bromo-3-[(Z)-1-cyano-2-(2-cyano-5-methoxy-phenyl)vinyl]indole-1-carboxylate

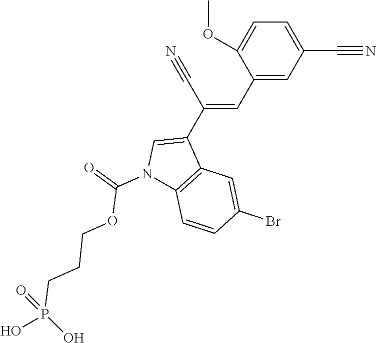 [SMILES: COc1ccc(C#N)cc1/C=C(\C#N)c1cn(C(=O)OCCCP(=O)(O)O)c2ccc(Br)cc12]

### A) Dibenzyl (3-hydroxypropyl)phosphonate

Dibenzyl phosphite (4.4 mL, 20 mmol) was added to a stirred suspension of HNa (1 g, 25 mmol) in dry DMF under nitrogen at 0° C. After 15 min at RT, the solution was cooled to 0° C. then 2-(3-Bromopropoxy)tetrahydro-2H-pyran (3.4 mL, 20 mmol) was added and the reaction mixture stirred at RT for 5 h. The solution was diluted with water (100 mL) and extracted with diethyl ether (2×150 mL). The combined extracts were washed with aq. NH₄Cl (4×30 mL), brine (50 mL), dried over Na₂SO₄ and concentrated in vacuo. The residue was purified by flash chromatography on a SiO₂ column (eluent: 0 to 100% EtOAc in petroleum ether) to provide 5.41 g of a colorless liquid. Yield=67%

PPTS (0.025 g, 0.1 mmol) was added to a stirred solution of the previous compound (0.404 g, 1 mmol) in EtOH (10 mL) and the solution was heated at 55° C. for 3 h. The solution was concentrated in vacuo and the residue purified by flash chromatography on a SiO₂ column (eluent: 0 to 100% EtOAc in petroleum ether then 0 to 10% MeOH in EtOAc) to provide 0.264 g of colorless oil. Yield=82%

LC-MS: [M+H]=321

¹H NMR (300 MHz, CDCl₃) δ ppm: 7.35 (s, 10H), 5.07 (dd, J=11.7, 9 Hz, 2H), 4.97 (dd, J=11.7, 5.7 Hz, 2H), 3.65 (t, J=5.7 Hz, 2H), 1.96-1.74 (m, 4H)

### B) 3-[dibenzyloxy(dioxo)-lambda6-phosphanyl]propyl 5-bromo-3-[(Z)-1-cyano-2-(2-cyano-5-methoxy-phenyl)vinyl]indole-1-carboxylate

Under N₂, dibenzyl (3-hydroxypropyl)phosphonate (0.042 g, 0.132 mmol) was dissolved in DCM (1 mL) and the solution cooled to 0° C. Triphosgene (14 mg, 0.049 mmol) was added followed by DIPEA (0.023 mL, 0.132 mmol). The solution was stirred for 1 h then at RT for 1 h. In a separate flask, (Z)-3-(2-(5-bromo-1H-indol-3-yl)-2-cyanovinyl)-4-methoxybenzonitrile (0.05 g, 0.132 mmol) was dissolved in DMF (1 mL) under N₂ and cooled to 0° C. HNa (0.063 g, 0.158 mmol) was added and the solution stirred at RT for 1 h. To the indole solution was added dropwise the chloroformate solution at RT. The reaction mixture was stirred at RT for 2 h then quenched by addition of saturated aq. NH₄Cl (5 mL) and extracted by EtOAc (20 mL). The organic layer was washed with sat. aq. NH₄Cl (3×5 mL) dried over Na₂SO₄ and concentrated in vacuo. The residue was purified by flash chromatography on a SiO₂ column (eluent: 0 to 100% EtOAc in petroleum ether) to give 0.041 g of a pale yellow solid. Yield=43%

¹H NMR (300 MHz, DMSO) δ ppm: 8.27 (d, J=1.8 Hz, 1H), 8.17 (d, J=1.8 Hz, 1H), 8.14 (s, 1H), 8.11 (s, 1H), 8.0 (dd, J=1.8, 8.7 Hz, 1H), 7.92 (s, 1H), 7.61 (dd, J=1.8, 9.0 Hz, 1H), 7.44-7.25 (m, 11H), 5.11-4.93 (m, 4H), 4.45 (t, J=5.7 Hz, 2H), 3.97 (s, 3H) 2.15-1.90 (m, 4H)

### C) 3-[hydroxy(dioxo)-lambda6-phosphanyl]propyl 5-bromo-3-[(Z)-1-cyano-2-(2-cyano-5-methoxy-phenyl)vinyl]indole-1-carboxylate

3-[dibenzyloxy(dioxo)-lambda6-phosphanyl]propyl 5-bromo-3-[(Z)-1-cyano-2-(2-cyano-5-methoxy-phenyl)vinyl]indole-1-carboxylate (0.348 g, 0.48 mmol) was dissolved in 4M HCl in dioxane (9.5 mL) and the solution was stirred at 90° C. for 2 h. The solution was concentrated under vacuum and the residue was triturated in DCM to precipitate the product. The solid was filtered and dried under vacuum to give 0.21 g of the desired compound as a white solid. Yield =80%

LC-MS: [M−H]=542

¹H NMR (400 MHZ, DMSO) δ ppm: 8.26 (s, 1H), 8.14 (m, 3H), 7.99 (d, J=8.4 Hz, 1H), 7.92 (s, 1H), 7.64 (d, J=9.0 Hz, 1H), 7.36 (d, J=8.7 Hz, 1H), 4.49 (t, J=6.3 Hz, 2H), 3.98 (s, 3H), 2.01 (m, 2H), 1.70 (q, J=8.2 Hz, 2H)

³¹P NMR (162 MHZ, DMSO d₆, δ ppm: 25.44

### Example 18:4-[hydroxy(dioxo)-lambda6-phosphanyl]butyl 5-bromo-3-[(Z)-1-cyano-2-(2-cyano-5-methoxy-phenyl)vinyl]indole-1-carboxylate

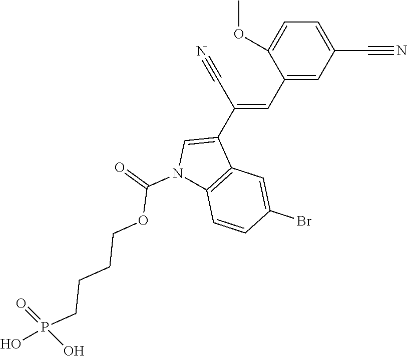 [SMILES: COc1ccc(C#N)cc1/C=C(\C#N)c1cn(C(=O)OCCCCP(=O)(O)O)c2ccc(Br)cc12]

### A) 2-(4-Bromobutoxy)tetrahydro-2H-pyran

To an ice cooled of 4-bromobutanol (0.153 g, 1 mmol) in DCM (2 mL) were successively added DHP (0.091 mL, 1.1 mmol) and PTSA (0.002 g, 0.01 mmol). The solution was stirred at RT for 2 h, diluted with DCM (15 mL), washed with sat. NaHCO₃ (10 mL), brine (10 mL), dried and concentrated to dryness. The residue was purified by flash chromatography on a SiO₂ column (eluent 0 to 10% EtOAc in petroleum ether) to give 0.145 g of a clear liquid. Yield=61%

¹H NMR (300 MHZ, CDCl₃) δ ppm: 4.68-4.54 (m, 1H), 3.96-3.66 (m, 2H), 3.64-3.32 (m, 4H), 2.12-1.44 (m, 10H)

### B) Dibenzyl (4-(tetrahydropyran-2-yloxy)butyl)phosphonate

Dibenzyl phosphite (1.56 mL, 7 mmol) was added to a stirred suspension of HNa (0.35 g, 8.75 mmol) in dry DMF (10.5 mL) under nitrogen at 0° C. After 15 min at RT, the solution was cooled to 0° C. then 2-(4-Bromobutoxy)tetrahydro-2H-pyran (1.67 g, 7 mmol) in dry DMF (4 mL) was added and the reaction mixture stirred at RT for 3 h. The solution was diluted with water (20 mL) and extracted with diethyl ether (2×70 mL). The combined extracts were washed with aq. NH₄Cl (4×20 mL), dried over Na₂SO₄ and concentrated in vacuo. The residue was purified by flash chromatography on a SiO₂ column (eluent: 10 to 100% EtOAc in petroleum ether) to provide 1.71 g of a colorless liquid. Yield=58%

¹H NMR (300 MHZ, CDCl₃) δ ppm: 7.35 (s, 10H), 5.06 (dd, J=11.7, 8.7 Hz, 2H), 4.97 (dd, J=11.7, 8.1 Hz, 2H), 4.54 (t, J=3.6 Hz, 1H), 3.88-3.77 (m, 1H), 3.75-3.65 (m, 1H), 3.54-3.43 (m, 1H), 3.39-3.28 (m, 1H), 1.94-1.40 (m, 12H)

### C) Dibenzyl (4-hydroxybutyl)phosphonate

PPTS (0.086 g. 0.34 mmol) was added to a stirred solution of the previous compound (1.44 g, 3.44 mmol) in EtOH (34 mL) and the solution was heated at 55° C. for 4 h. The solution was concentrated in vacuo and the residue purified by flash chromatography on a SiO2 column (eluent: 0 to 10% MeOH in DCM) to provide 1.1 g of colorless liquid. Yield=95%

### D) 4-[dibenzyloxy(dioxo)-lambda6-phosphanyl]butyl 5-bromo-3-[(Z)-1-cyano-2-(2-cyano-5-methoxy-phenyl)vinyl]indole-1-carboxylate

Under N₂, dibenzyl (4-hydroxybutyl)phosphonate (0.265 g, 0.796 mmol) was dissolved in DCM (6 mL) and the solution cooled to 0° C. Triphosgene (87 mg. 0.295 mmol) was added followed by DIPEA (0.139 mL, 0.796 mmol). The solution was stirred for 1 h then at RT for 1 h. In a separate flask. (Z)-3-(2-(5-bromo-1H-indol-3-yl)-2-cyanovinyl)-4-methoxybenzonitrile (0.3 g. 0.796 mmol) was dissolved in dry DMF (6 mL) under N₂ and cooled to 0° C. HNa (0.038 g, 0.955 mmol) was added and the solution stirred at RT for 1 h. To the indole solution was added dropwise the chloroformate solution at RT. The reaction mixture was stirred at RT overnight, then quenched by addition of saturated aq. NH₄Cl (30 mL) and extracted by EtOAc (120 mL). The organic layer was washed with sat. aq. NH₄Cl (3×30 mL) dried over Na₂SO₄ and concentrated under reduced pressure. The residue was purified by flash chromatography on a SiO₂ column (eluent: 0 to 100% EtOAc in petroleum ether) to give 0.342 g of a pale yellow solid. Yield=43%

### E) 4-[hydroxy(dioxo)-lambda6-phosphanyl]butyl 5-bromo-3-[(Z)-1-cyano-2-(2-cyano-5-methoxy-phenyl)vinyl]indole-1-carboxylate

4-[dibenzyloxy(dioxo)-lambda6-phosphanyl]butyl 5-bromo-3-[(Z)-1-cyano-2-(2-cyano-5-methoxy-phenyl)vinyl]indole-1-carboxylate (0.334 g. 0.452 mmol) was dissolved in 4M HCl in dioxane (9.5 mL) and the solution was stirred at 90° C. for 2 h. The solution was concentrated under vacuum and the residue was triturated in DCM to precipitate the product. The solid was collected by filtration and dried under vacuum to give 0.222 g of the desired compound as a white solid. Yield=88%

LC-MS: [M−H]=557.8

¹H NMR (300 MHz, DMSO) δ ppm: 8.26 (s, 1H), 8.12 (m, 3H), 7.99 (d, J=8.4 Hz, 1H), 7.92 (s, 1H), 7.63 (d, J=8.7 Hz, 1H), 7.36 (d, J=9.0 Hz, 1H), 4.46 (t. J=5.4 Hz, 2H), 3.98 (s, 3H), 1.90 (m, 2H), 1.63 (m, 4H)

³¹P NMR (162 MHZ, DMSO, δ ppm: 26.18

### II. L₁ Prodrugs

### Example 19: (2S,3S,4S,5R,6S)-6-[4-[[5-bromo-3-[(Z)-1-cyano-2-(5-cyano-2-methoxy-phenyl)vinyl]indole-1-carbonyl]oxymethyl]phenoxy]-3,4,5-trihydroxy-tetrahydropyran-2-carboxylic acid

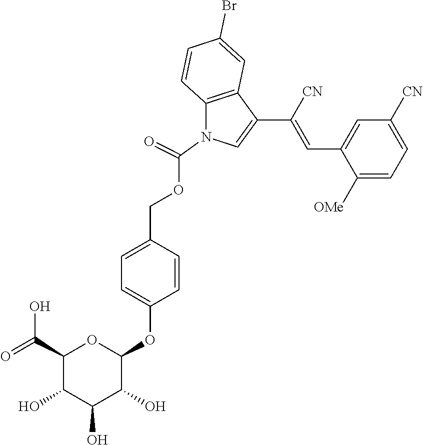 [SMILES: COc1ccc(C#N)cc1/C=C(\C#N)c1cn(C(=O)OCc2ccc(O[C@@H]3O[C@H](C(=O)O)[C@@H](O)[C@H](O)[C@H]3O)cc2)c2ccc(Br)cc12]

### 1) Methyl (2S,3S,4S,5R,6S)-3,4,5-triacetoxy-6-[4-(hydroxymethyl)phenoxy]tetrahydropyran-2-carboxylate

The compound was synthesized in two steps according to Yu-Ling Leu et al. J. Med. Chem. 2008, 51, 1470-1746

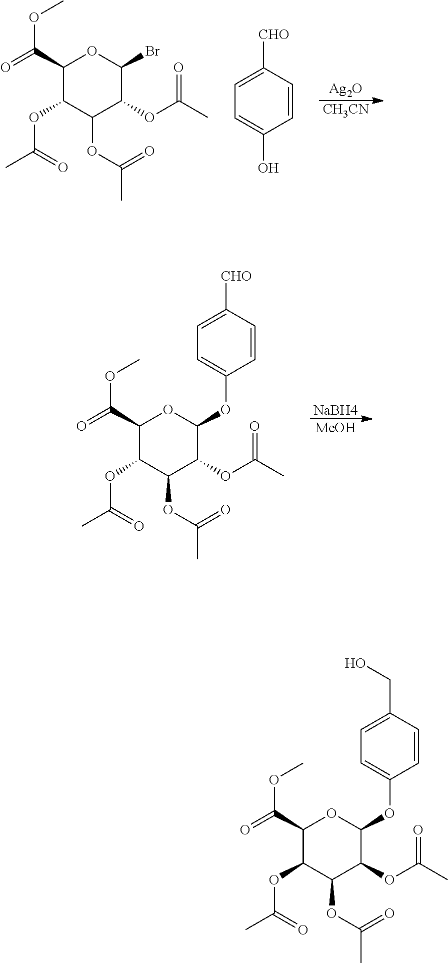

### a) Methyl (2S,3S,4S,5R,6S)-3,4,5-triacetoxy-6-(4-formylphenoxy)tetrahydropyran-2-carboxylate

In a 100 mL round bottom flask equipped with magnetic stirring, methyl acetobromo-a-D-glucuronic acetate (2.00 g, 5.04 mmol) was dissolved in CH₃CN (40 mL), then 4-hydroxybenzaldehyde (584 mg, 4.78 mmol) and Ag₂O (1.11 g, 4.78 mmol) were slowly added. The mixture was stirred at RT for 18 h. The insoluble material was filtered off. The filtrate was evaporated under reduced pressure to give a dark brown oil. The crude oil was triturated with EtOH (10 mL) to give 1.64 g of a brown solid. The solid was dissolved in DCM (50 mL), purified by chromatography on a SiO₂ cake, elution from DCM 100% then from DCM/MeOH (98/2) to give 1.10 g of methyl (2S,3S,4S,5R,6S)-3,4,5-triacetoxy-6-(4-formylphenoxy)tetrahydropyran-2-carboxylate as an off-white solid (yield=50%).

LCMS-ESI: [M+H+18]⁺=456

### b) Methyl (2S,3S,4S,5R,6S)-3,4,5-triacetoxy-6-[4-(hydroxymethyl)phenoxy]tetrahydropyran-2-carboxylate

In a 250 mL round bottom flask, methyl(2S,3S,4S,5R,6S)-3,4,5-triacetoxy-6-(4-formylphenoxy)tetrahydropyran-2-carboxylate (1.10 g, 2.52 mmol) was dissolved in CHCl₃ (94 mL) and 2-propanol (16 mL). SiO₂ (5.50 g) was added. Then NaBH4 (95 mg, 2.52 mmol) was slowly added at 5° C. The mixture was stirred at +5° C. for 2 h, hydrolyzed with 200 ml of H₂O, filtered to remove SiO₂ and extracted with DCM. The organic layer was dried over MgSO₄, evaporated under reduce pressure to give a colorless oil that was triturated with Ethanol (5 mL) to give 1.00 g of methyl(2S,3S,4S,5R,6S)-3,4,5-triacetoxy-6-[4-(hydroxymethyl)phenoxy]tetrahydropyran-2-carboxylate as a white solid (yield=90%).

LCMS-ESI: [M+H+18]⁺=458

### 2) Methyl(2S,3S,4S,5R,6S)-3,4,5-triacetoxy-6-[4-[(4-nitrophenoxy)carbonyloxymethyl]phenoxy]tetrahydropyran-2-carboxylate

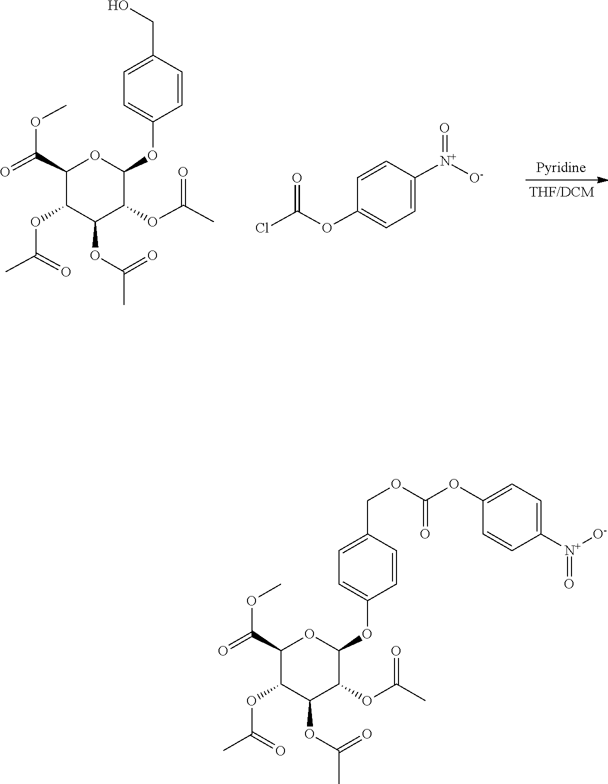 [SMILES: COC(=O)[C@H]1O[C@@H](Oc2ccc(CO)cc2)[C@H](OC(C)=O)[C@@H](OC(C)=O)[C@@H]1OC(C)=O ; O=C(Cl)Oc1ccc([N+](=O)[O-])cc1 ; COC(=O)[C@H]1O[C@@H](Oc2ccc(COC(=O)Oc3ccc([N+](=O)[O-])cc3)cc2)[C@H](OC(C)=O)[C@@H](OC(C)=O)[C@@H]1OC(C)=O]

In 100 ml pear flask equipped with magnetic stirring, methyl (2S,3S,4S,5R,6S)-3,4,5-triacetoxy-6-[4-(hydroxymethyl)phenoxy]tetrahydropyran-2-carboxylate (1.00 g, 2.27 mmol) was dissolved in dried THF (30 mL) and DCM (15 mL). 4-nitrophenylchloroformate (687 mg, 3.41 mmol) was slowly added at RT. After 5 min, pyridine (286 μL, 3.41 mmol) was added and the reaction mixture was stirred at 20° C. for 2 h. Ethyl Acetate (150 mL) and aqueous solution of 10% citric acid (150 mL) were added to the reaction mixture. The organic layer was separated, washed with water and brine, dried over magnesium sulfate and the solvent removed under vacuum to give 1.6 g of pale yellow oil. The crude oil was triturated with EtOH (5 mL) to give 1.64 g of methyl(2S,3S,4S,5R,6S)-3,4,5-triacetoxy-6-[4-[(4-nitrophenoxy)carbonyloxymethyl]phenoxy]tetrahydropyran-2-carboxylate as a pale yellow oil (yield=92%).

LCMS-ESI: [M+H+18]⁺=623

### 3) [4-[(2S,3R,4S,5S,6S)-3,4,5-triacetoxy-6-methoxycarbonyl-tetrahydropyran-2-yl]oxyphenyl]methyl 5-bromo-3-[(Z)-1-cyano-2-(5-cyano-2-methoxy-phenyl)vinyl]indole-1-carboxylate

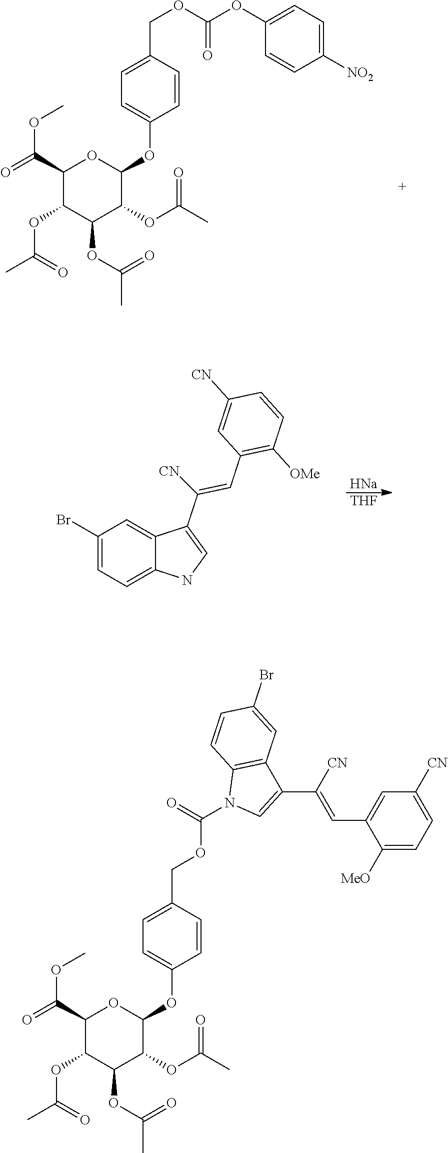

In a 15 mL reactor equipped with magnetic stirring, (Z)-3-(2-(5-bromo-1H-indol-3-yl)-2-cyanovinyl)-4-methoxybenzonitrile (250 mg, 0.66 mmol) was charged in THF (10 mL). At +5° C., HNa 60% (26 mg, 0.66 mmol) was slowly added under nitrogen atmosphere. The reaction was stirred at +5° C. for 10 mn. In a separate 100 ml round bottom flask equipped with magnetic stirring, under inert atmosphere, methyl (2S,3S,4S,5R,6S)-3,4,5-triacetoxy-6-[4-[(4-nitrophenoxy)carbonyloxymethyl]phenoxy]tetrahydropyran-2-carboxylate (400 mg, 0.66 mmol) was dissolved in THF (25 mL). To this solution was slowly added at 5° C. the previous solution of indole sodium salt in THF. Then the reaction mixture was stirred at RT for 2 h. An aqueous solution of citric acid (150 mL) was slowly added, the mixture stirred for 18 h, the precipitated solid was filtered, washed with water to give 580 mg of a yellow solid. The crude solid was directly purified by flash chromatography on SiO₂ column (24 g), eluted with a gradient from heptane 100% to AcOEt 100% to give 377 mg of [4-[(2S,3R,4S,5S,6S)-3,4,5-triacetoxy-6-methoxycarbonyl-tetrahydropyran-2-yl]oxyphenyl]methyl5-bromo-3-[(Z)-1-cyano-2-(5-cyano-2-methoxy-phenyl)vinyl]indole-1-carboxylate as an off-white solid (Yield=67%).

LCMS-ESI: [M+H+18]⁺=863

¹H NMR (400 MHZ, DMSO-d₆) δ ppm: 8.25 (s, 1H), 8.18-8.10 (m, 3H), 7.99 (d, 1H), 7.93 (s, 1H), 7.63 (d, 1H), 7.54 (d, 2H), 7.34 (d, 1H), 7.05 (d, 2H), 5.70 (d, 1H), 5.46 (brs, 3H), 5.10-5.03 (m, 2H), 4.71 (d, 1H), 3.96 (s, 3H), 3.62 (s, 3H), 2.01 (s, 9H)

### 4) (2S,3S,4S,5R,6S)-6-[4-[[5-bromo-3-[(Z)-1-cyano-2-(5-cyano-2-methoxy-phenyl)vinyl]indole-1-carbonyl]oxymethyl]phenoxy]-3,4,5-trihydroxy-tetrahydropyran-2-carboxylic acid

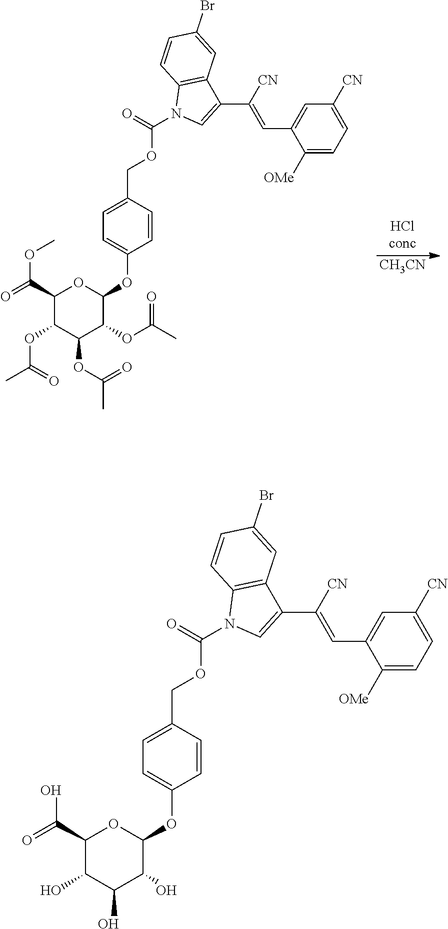 [SMILES: COC(=O)[C@H]1O[C@@H](Oc2ccc(COC(=O)n3cc(/C(C#N)=C/c4cc(C#N)ccc4OC)c4cc(Br)ccc43)cc2)[C@H](OC(C)=O)[C@@H](OC(C)=O)[C@@H]1OC(C)=O ; "CH3CN" ; COc1ccc(C#N)cc1/C=C(\C#N)c1cn(C(=O)OCc2ccc(O[C@@H]3O[C@H](C(=O)O)[C@@H](O)[C@H](O)[C@H]3O)cc2)c2ccc(Br)cc12]

In 10 ml reactor equipped with magnetic stirring, [4-[(2S,3R,4S,5S,6S)-3,4,5-triacetoxy-6-methoxycarbonyl-tetrahydropyran-2-yl]oxyphenyl]methyl5-bromo-3-[(Z)-1-cyano-2-(5-cyano-2-methoxy-phenyl)vinyl]indole-1-carboxylate (150 mg, 0.169 mmol) was charged in acetonitrile (1.7 mL). Concentrated HCl (1.7 mL, 16.9 mmol) was added and the reaction mixture was heated at 40° C. for 2 days. The mixture was concentrated under reduce pressure and the residue was purified by flash chromatography on a SiO₂ cake, elution DCM 100% to DCM/MeOH (95/5), then AcOEt/MeOH/AcCN/H2O (7/1/1/1) to give a yellow solid, which was purified again on a SiO₂ cake, elution with the mixture AcOEt/MeOH/AcCN/H2O (7/1/1/1) to give 12 mg of (2S,3S,4S,5R,6S)-6-[4-[[5-bromo-3-[(Z)-1-cyano-2-(5-cyano-2-methoxy-phenyl)vinyl]indole-1-carbonyl]oxymethyl]phenoxy]-3,4,5-trihydroxy-tetrahydropyran-2-carboxylic acid as a yellow solid (yield=10%)

LCMS-ESI: [M+H+18]⁺=723

### Example 22: [4-[[(2S)-2-[[(2S)-2-[3-[2-[3-(2,5-dioxopyrrol-1-yl)propanoylamino](peg4)propanoylamino]-3-methyl-butanoyl]amino]-5-ureido-pentanoyl]amino]phenyl]methyl-5-bromo-3-[(Z)-1-cyano-2-(5-cyano-2-methoxy-phenyl)vinyl]indole-1-carboxylate

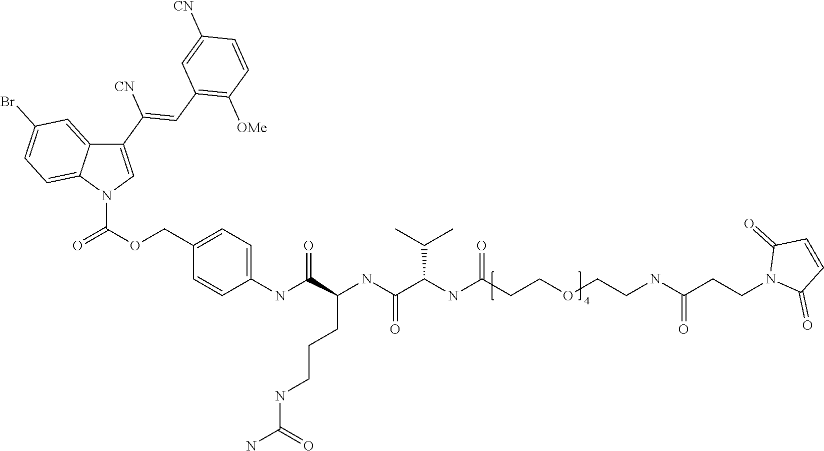

### 1) Example 20: [4-[[(2S)-2-[[(2S)-2-(tert-butoxycarbonylamino)-3-methyl-butanoyl]amino]-5-ureido-pentanoyl]amino]phenyl]methyl-5-bromo-3-[(Z)-1-cyano-2-(5-cyano-2-methoxy-phenyl)vinyl]indole-1-carboxylate

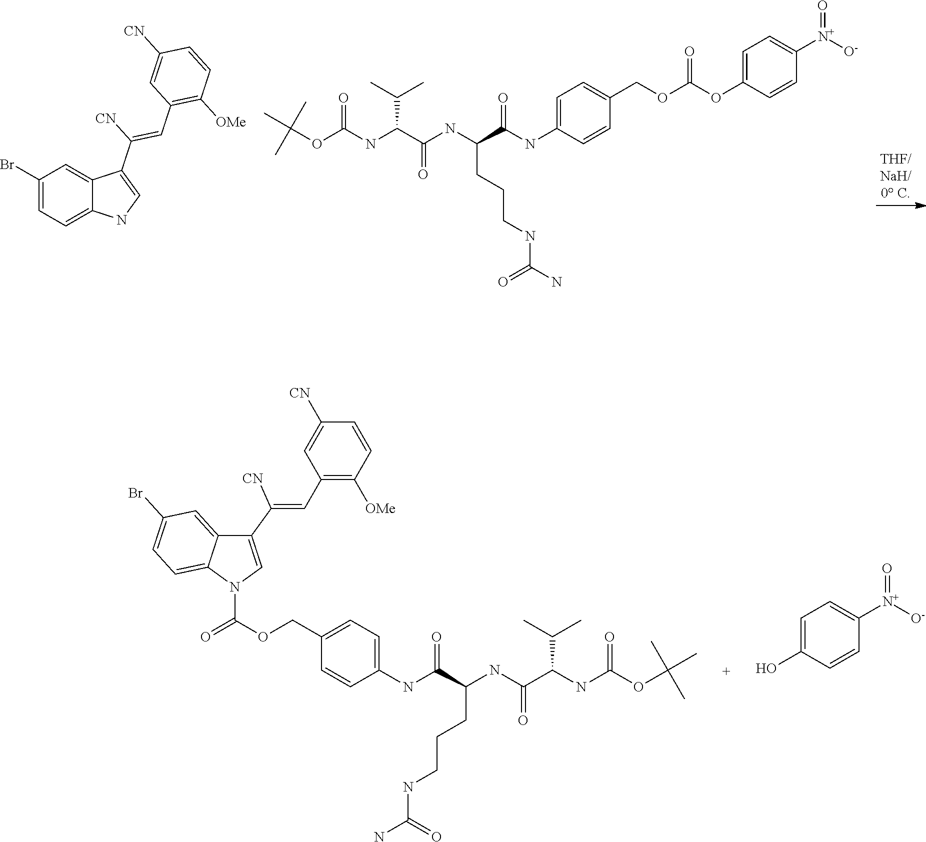

In a 50 ml pear-shaped flask under argon atmosphere, (Z)-3-(2-(5-bromo-1H-indol-3-yl)-2-cyanovinyl)-4-methoxybenzonitrile (140 mg, 0.37 mmol) was solubilized in dried THF (6 mL). HNa 60% (15.6 mg, 0.39 mmol) was added at 5° C. to give a red-orange solution; then the solution was stirred at RT for 10 min. The solution was cooled again at 5° C. then Boc-Val-Cit-PABA-PNP (Iris Biotech, 240 mg, 0.37 mmol) dissolved in 15 mL of THF was added. After 30 min an orange gel occurred with a yellow precipitate below. After 3 hours water was slowly added and the aqueous solution extracted with ethyl acetate. The organic layer was washed with water and brine, dried over magnesium sulfate and concentrated to dryness to give 400 mg of crude product. The crude product was suspended in water, heated at 50° C. for 15 min. 4-nitro-phenol was filtrated off and the filtrate concentrated under vacuum. The resulting solid was suspended in MeOH (20 mL), heated at 50° C. for 15 min and filtered to give 230 mg of a pale yellow solid. The solvent of the filtrate was removed and the resulting solid residue was triturated with few MeOH to give a second batch of 50 mg of [4-[[(2S)-2-[[(2S)-2-(tert-butoxycarbonylamino)-3-methyl-butanoyl]amino]-5-ureido-pentanoyl]amino]phenyl]methyl-5-bromo-3-[(Z)-1-cyano-2-(5-cyano-2-methoxy-phenyl)vinyl]indole-1-carboxylate as pale yellow solid (total yield=85%).

LCMS-ESI: [M+H-Boc]⁺=783

¹H NMR (400 MHZ, DMSO-d₆) δ ppm: 10.19 (s, 1H), 8.27 (s, 1H), 8.20 (s, 1H), 8.14 (d, J=12 Hz, 2H), 8.04 (t, J=12 Hz, 2H), 8.0 (s, 1H), 7.69 (t, 3H), 7.52 (d, 2H), 7.38 (d, J=8 Hz, 1H), 6.78 (d, J=12 Hz, 1H), 5.99 (brs, 1H), 5.45 (d, J=12 Hz, 4H), 4.46 (brs, 1H), 3.99 (s, 3H), 3.85 (brs, 1H), 3.0 (brd, 2H), 1.97 (m, 1H), 1.65 (m, 2H), 1.39 (m, 11H), 0.85 (dd, J=8, 16 Hz, 6H)

### 2) Example 21: [4-[[(2S)-2-[[(2S)-2-aminopropanoyl]amino]-5-ureido-pentanoyl]amino]phenyl]methyl-5-bromo-3-[(Z)-1-cyano-2-(5-cyano-2-methoxy-phenyl)vinyl]indole-1-carboxylate hydrochloride

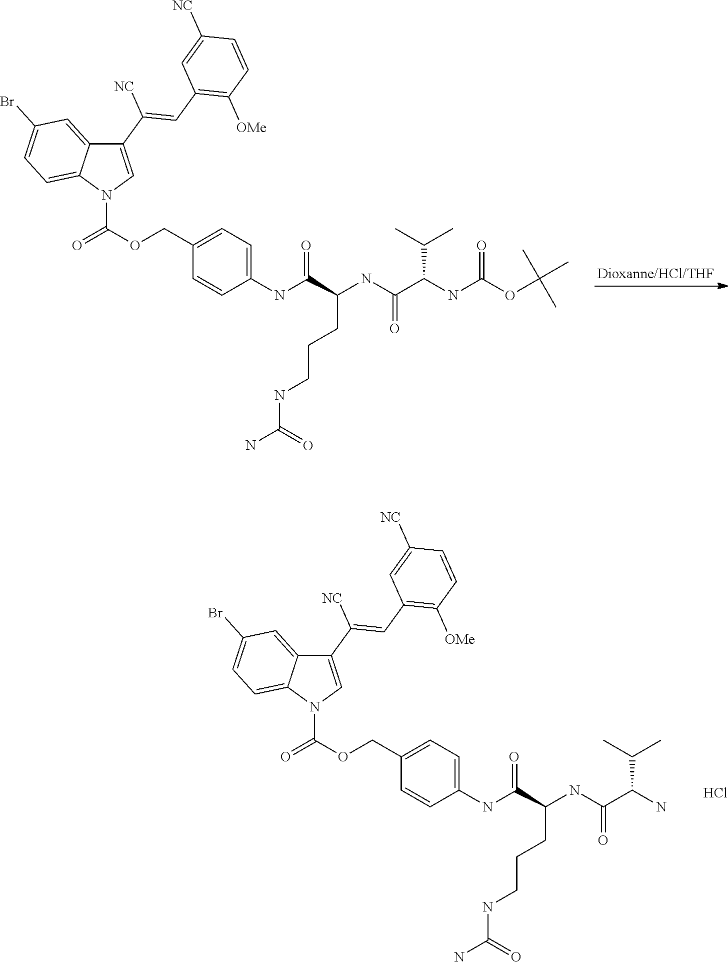

In a 50 ml pear flask under argon atmosphere, to a solution of the previous compound (380 mg, 0.43 mmol) in 20 mL of THF was added a solution of 4N HCl in dioxane (3.23 mL, 12.92 mmol). The reaction mixture was stirred at 20° C. for 18 h. The solvent was removed under vacuum to give a solid which was triturated with THF (2 mL), filtered to give 210 mg of [4-[[(2S)-2-[(2S)-2-aminopropanoyl]amino]-5-ureido-pentanoyl]amino]phenyl]methyl-5-bromo-3-[(Z)-1-cyano-2-(5-cyano-2-methoxy-phenyl)vinyl]indole-1-carboxylate hydrochloride as a pale yellow powder (yield=92%).

LCMS-ESI: [M+H]⁺=783

¹H NMR (400 MHZ, DMSO-d₆) δ ppm: 10.39 (s, 1H), 8.76 (d, J=8 Hz, 1H), 8.19-7.94 (m, 8H), 7.66 (t, 3H), 7.52 (d, 2H), 7.36 (d, J=12 Hz, 1H), 5.46 (s, 2H), 4.52 (brs, 1H), 4.11 (brs, 3H), 3.97 (s, 3H), 3.68 (brs, 1H), 3.01 (brd, 2H), 2.09 (brs, 1H), 1.65 (brs, 2H), 1.47 (brs, 2H), 0.95 (d, J=8 Hz, 6H)

### 3) Example 22: [4-[[(2S)-2-[[(2S)-2-[3-[2-[3-(2,5-dioxopyrrol-1-yl)propanoylamino]ethoxy](peg4) propanoylamino]-3-methyl-butanoyl]amino]-5-ureido-pentanoyl]amino]phenyl]methyl 5-bromo-3-[(Z)-1-cyano-2-(5-cyano-2-methoxy-phenyl)vinyl]indole-1-carboxylate

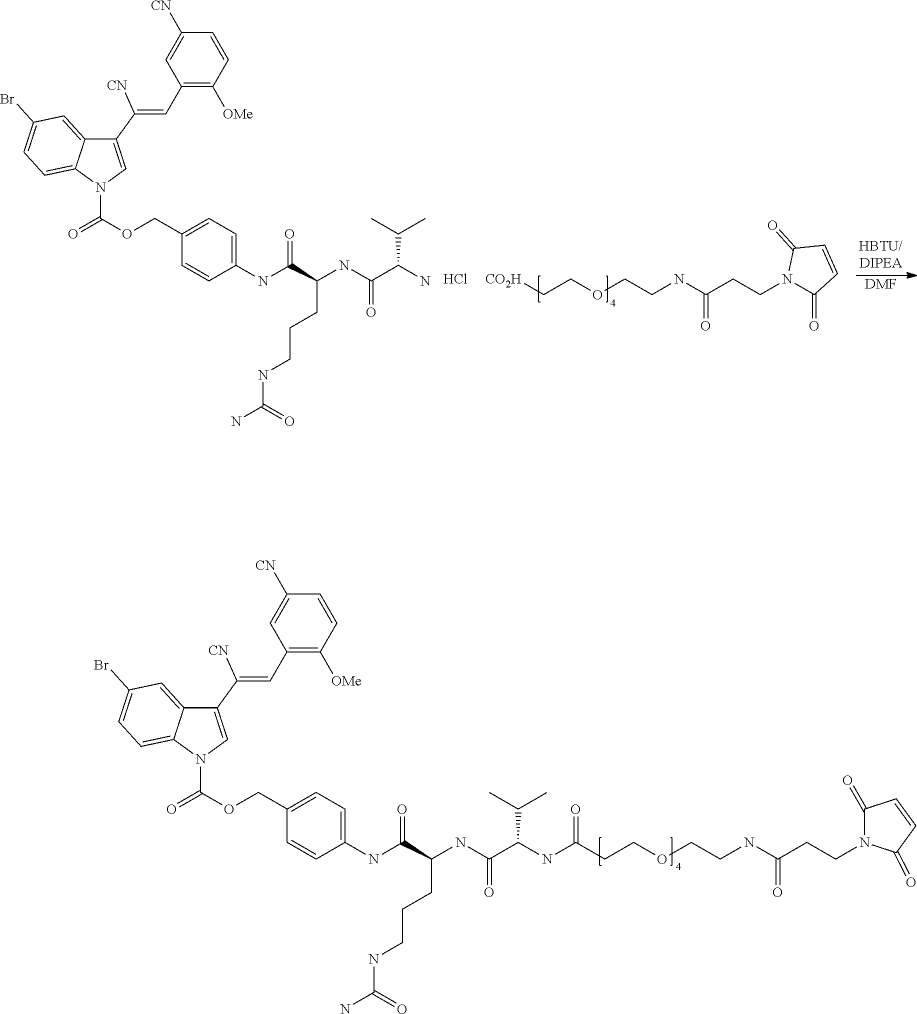

In a 15 ml reactor equipped with magnetic stirring, 19-maleimido-17-oxo-4,7,10,13-tetraoxa-16-azanonadecanoic acid (122 mg. 0.29 mmol) and HBTU (185 mg, 0.49 mmol) were charged in DMF (4 mL). At +5° C., DIPEA (148 μL, 0.85 mmol) was added. The solution was stirred at +5° C. for 20 min and [4-[[(2S)-2-[[(2S)-2-aminopropanoyl]amino]-5-ureido-pentanoyl]amino]phenyl]methyl-5-bromo-3-[(Z)-1-cyano-2-(5-cyano-2-methoxy-phenyl)vinyl]indole-1-carboxylate hydrochloride (200 mg. 0.24 mmol) was added. The reaction was stirred at RT for 18 h. The mixture was slowly poured in water (25 mL) under stirring, the precipitate was filtered, washed with water to give a brown solid. The crude solid was directly purified by flash chromatography on SiO₂ column (12 g), solid loading, eluted with gradient DCM 100% to DCM/MeOH (8/2) to give 85 mg of [4-[[(2S)-2-[(2S)-2-[3-[2-[3-(2,5-dioxopyrrol-1-yl)propanoylamino]ethoxy](peg4) propanoylamino]-3-methyl-butanoyl]amino]-5-ureido-pentanoyl]amino]phenyl]methyl-5-bromo-3-[(Z)-1-cyano-2-(5-cyano-2-methoxy-phenyl)vinyl]indole-1-carboxylate as a yellow solid (yield=29%).

LCMS-ESI: [M+H]⁺=1184

1H NMR (400 MHZ, DMSO-d₆) δ 10.09 (s, 1H), 8.18 (s, 1H), 8.15-7.88 (m, 7H), 7.66 (t, 3H), 7.50 (d, 2H), 7.36 (d, 1H), 7.00 (s, 2H), 5.98 (brs, 1H), 5.43 (d, 4H), 4.37 (brs, 1H), 4.22 (t. 1H), 3.96 (s, 3H), 3.58 (t, 4H), 3.48 (s, 12H), 3.14 (m, 2H), 2.97 (d1, 2H), 2.32 (m, 2H), 1.95 (m, 1H), 1.60 (d1, 2H), 1.43 (d1, 2H), 0.84 (dd, 6H)

### Example 23: [4-[[(2S)-2-[[(2S)-2-[3-[2-[3-(2,5-dioxopyrrol-1-yl)propanoylamido](peg24) propanoylamino]-3-methyl-butanoyl]amino]-5-ureido-pentanoyl]amino]phenyl]methyl 5-bromo-3-[(Z)-1-cyano-2-(5-cyano-2-methoxy-phenyl)vinyl]indole-1-carboxylate

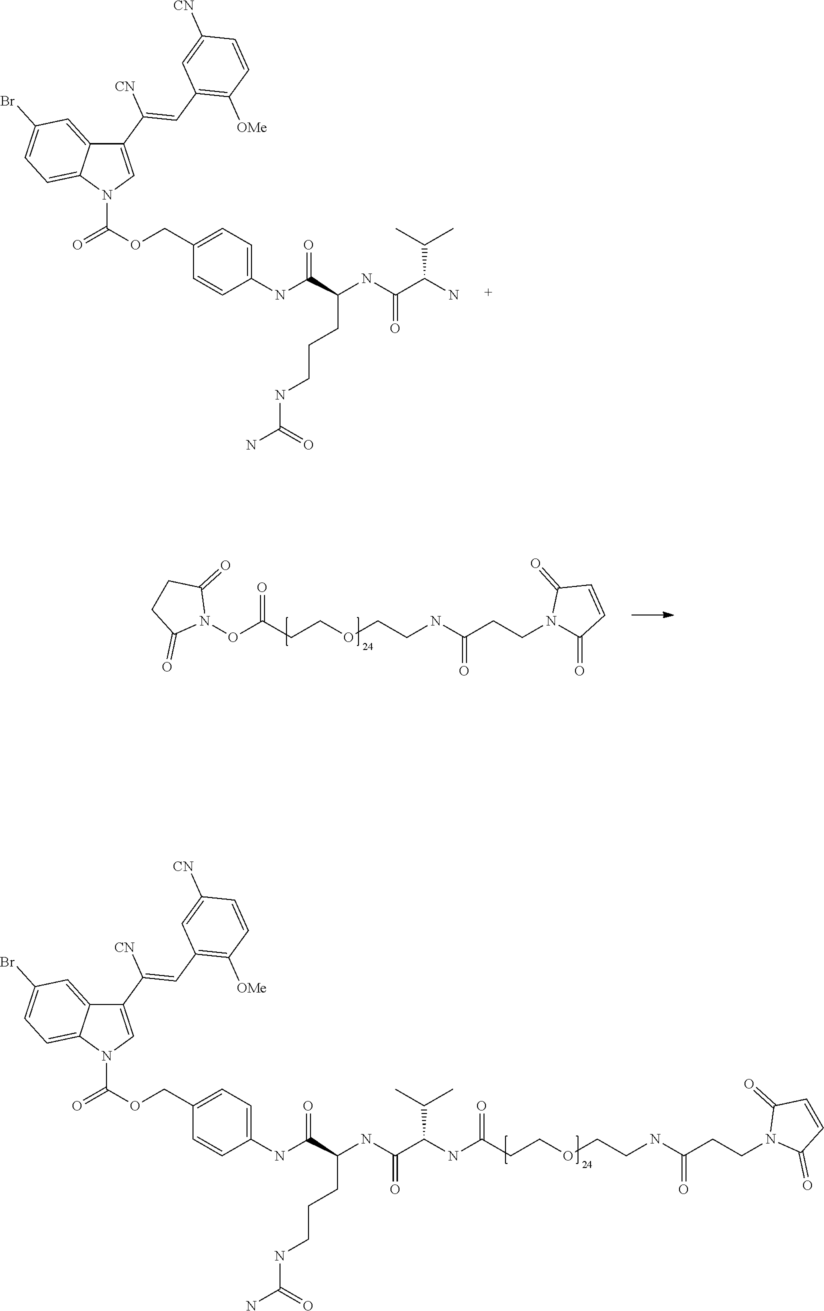

In a 15 ml reactor equipped with magnetic stirring, [4-[[(2S)-2-[[(2S)-2-aminopropanoyl]amino]-5-ureido-pentanoyl]amino]phenyl]methyl-5-bromo-3-[(Z)-1-cyano-2-(5-cyano-2-methoxy-phenyl)vinyl]indole-1-carboxylate hydrochloride (140 mg, 0.17 mmol) and diisopropylethylamine (59 μL, 0.34 mmol) were charged in DMF (2 mL). Mal-PEG (24)-NHS (Iris Biotech, 262 mg, 0.19 mmol was added and the reaction mixture stirred at RT for 1 h. The reaction mixture was triturated three times with DIPE (8 mL) to eliminate DMF and the resulting oil was suspended in 0.2N HCl (20 mL), extracted with DCM (4×15 mL). The organic phase was washed with water (15 mL), dried over Na₂SO₄ and concentrated under reduce pressure to give 350 mg of a yellow oil. The crude oil was directly purified twice by flash chromatography on a SiO₂ column, solid loading, eluted with a gradient from DCM 100% to DCM/MeOH 8/2 to give 100 mg of [4-[[(2S)-2-[(2S)-2-[3-[2-[3-(2,5-dioxopyrrol-1-yl)propanoylamido](peg24) propanoylamino]-3-methyl-butanoyl]amino]-5-ureido-pentanoyl]amino]phenyl]methyl-5-bromo-3-[(Z)-1-cyano-2-(5-cyano-2-methoxy-phenyl)vinyl]indole-1-carboxylate (yield=28%).

MS (MALDI-TOF): [M+Na]⁺=2083.91

### Example 26: (2R)-3-[[(1S)-1-[[(1S)-1-[[4-[5-bromo-3-[(Z)-1-cyano-2-(5-cyano-2-methoxy-phenyl)vinyl]indole-1-carbonyl]oxymethyl]phenyl]carbamoyl]-4-ureido-butyl]carbamoyl]-2-methyl-propyl]amino]-2-[3-[2-[3-(2,5-dioxopyrrol-1-yl)propanoylamino][peg4]propanoylamino]-3-oxo-propane-1-sulfonic acid

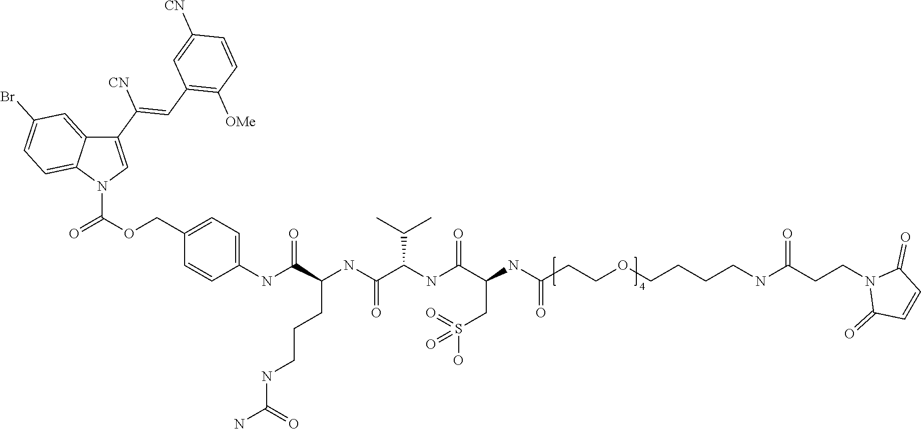

### 1) Example 24: 3-[(1S)-1-[[(1S)-1-[4-[5-bromo-3-[(Z)-1-cyano-2-(5-cyano-2-methoxy-phenyl)vinyl]indole-1-carbonyl]oxymethyl]phenyl]carbamoyl]-4-ureido-butyl]carbamoyl]-2-methyl-propyl]amino]-2-(tert-butoxycarbonylamino)-3-oxo-propane-1-sulfonic acid

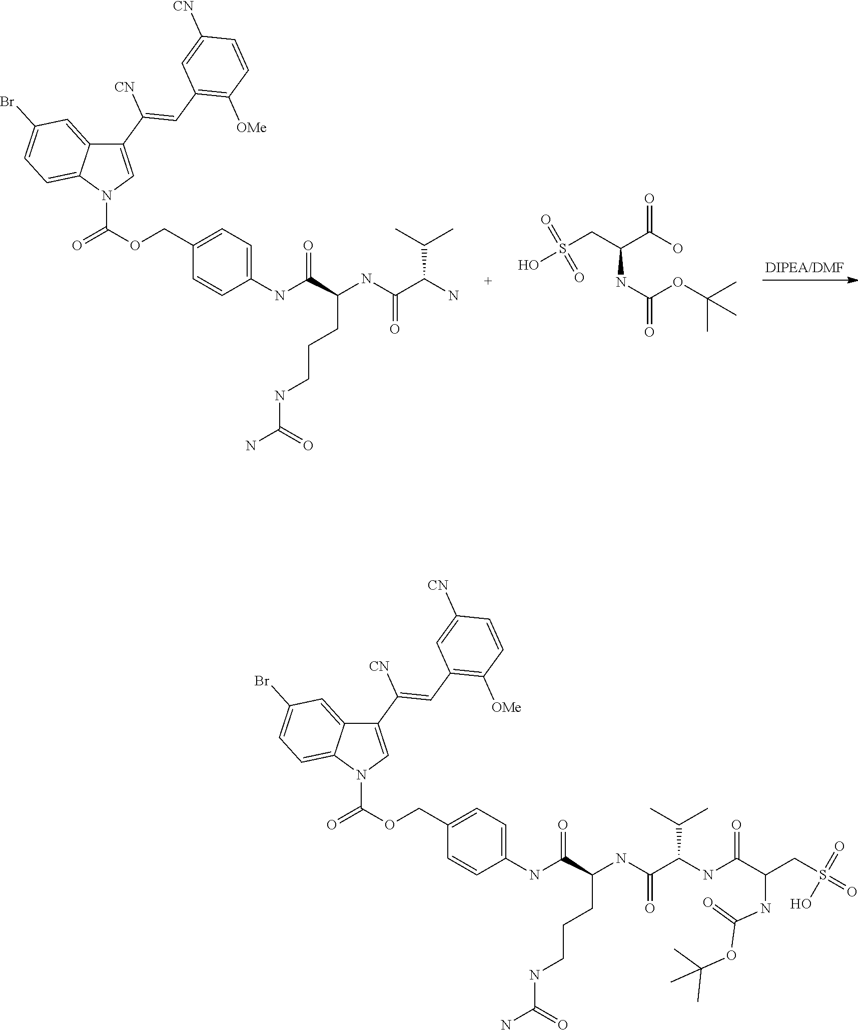

In a 15 mL reactor equipped with magnetic stirring, BOC-Cysteic acid, TEA (151 mg, 0.41 mmol) and HBTU (248 mg, 0.65 mmol) were charged in DMF (1 mL). Diisopropylethylamine (198 μL, 1.14 mmol) was added. The solution was stirred at RT for 30 min. [4-[(2S)-2-[[(2S)-2-aminopropanoyl]amino]-5-ureido-pentanoyl]amino]phenyl]methyl-5-bromo-3-[(Z)-1-cyano-2-(5-cyano-2-methoxy-phenyl)vinyl]indole-1-carboxylate, hydrochloride (268 mg, 0.33 mmol) was added at RT and the reaction stirred at RT for 18 h. The reaction mixture was poured in water, extracted with DCM, the organic layer washed with water, dried over Na₂SO₄ and concentrated under reduce pressure to give a brown oil. The crude oil was purified by flash chromatography on SiO₂ column (24 g), eluted with a gradient from DCM 100% to DCM/MeOH 3/1 to give 190 mg of 3-[[(1S)-1-[[(1S)-1-[[4-[[5-bromo-3-[(Z)-1-cyano-2-(5-cyano-2-methoxy-phenyl)vinyl]indole-1-carbonyl]oxymethyl]phenyl]carbamoyl]-4-ureido-butyl]carbamoyl]-2-methyl-propyl]amino]-2-(tert-butoxycarbonylamino)-3-oxo-propane-1-sulfonic acid as a pale yellow solid (yield=56%)

LCMS-ESI: [M−H]⁻=1034

¹H NMR (400 MHz, DMSO) δ ppm: 9.49 (s, 1H), 8.25 (s, 1H), 8.16 (s, 1H), 8.12 (m, 2H), 7.98 (d, 1H), 7.93 (s, 1H), 7.79 (d, 2H), 7.63 (d, 1H), 7.47 (d, 2H), 7.34 (d, 1H), 6.94 (d, 1H), 5.93 (brs, 1H), 5.46 (s, 2H), 5.42 (s, 2H), 4.37 (brs, 1H), 4.16 (brs, 1H), 3.97 (brs, 4H), 2.98 (brd, 4H), 2.13 (brs, 1H), 1.71 (brs, 2H), 1.33-1.35 (m, 11H), 1.22 (brs, 2H), 0.89 (d, J=8 Hz, 6H)

### 2) Example 25:2-amino-3-[[(1S)-1-[(1S)-1-[[4-[5-bromo-3-[(Z)-1-cyano-2-(5-cyano-2-methoxy-phenyl)vinyl]indole-1-carbonyl]oxymethyl]phenyl]carbamoyl]-4-ureido-butyl]carbamoyl]-2-methyl-propyl]amino]-3-oxo-propane-1-sulfonic acid

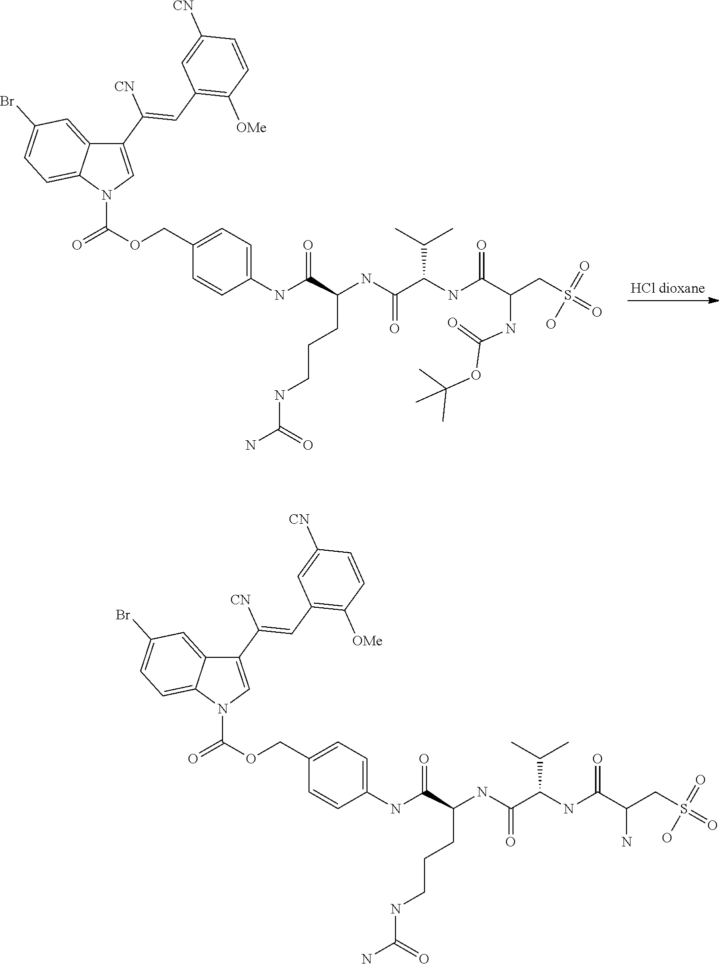

In a 15 mL reactor equipped with magnetic stirring, the previous compound (120 mg, 0.12 mmol) was charged in THF (2 mL). 4N HCl in dioxane (870 μL, 3.48 mmol) was added. The solution was heated at reflux for 5 min, cooled, the precipitate filtered, washed with THE to give 65 mg of 2-amino-3-[(1S)-1-[[(1S)-1-[[4-[[5-bromo-3-[(Z)-1-cyano-2-(5-cyano-2-methoxy-phenyl)vinyl]indole-1-carbonyl]oxymethyl]phenyl]carbamoyl]-4-ureido-butyl]carbamoyl]-2-methyl-propyl]amino]-3-oxo-propane-1-sulfonic acid as a yellow solid (yield=58%).

LCMS-ESI: [M−H]⁻=934

¹H NMR (400 MHZ, DMSO) δ ppm: 9.93 (s, 1H), 8.77 (d J=8 Hz, 1H), 8.25 (s, 2H), 8.14 (m, 2H), 7.98 (d, 2H), 7.94 (s, 1H), 7.70 (d, 2H), 7.50 (d, 2H), 7.34 (d, 1H), 6.05 (brs, 1H), 5.45 (s, 2H), 4.32-4.15 (m, 3H), 3.96 (s, 3H), 3.56 (brs, 4H), 2.98 (m, 4H), 2.13 (brs, 1H), 1.75 (brs, 2H), 1.35-1.25 (m, 2H), 0.87 (d, J=8 Hz, 6H)

### 3) Example 26: (2R)-3-[[(1S)-1-[[(1S)-1-[[4-[[5-bromo-3-[(Z)-1-cyano-2-(5-cyano-2-methoxy-phenyl)vinyl]indole-1-carbonyl]oxymethyl]phenyl]carbamoyl]-4-ureido-butyl]carbamoyl]-2-methyl-propyl]amino]-2-[3-[2-[3-(2,5-dioxopyrrol-1-yl)propanoylamino]ethoxy]propanoylamino]-3-oxo-propane-1-sulfonic acid

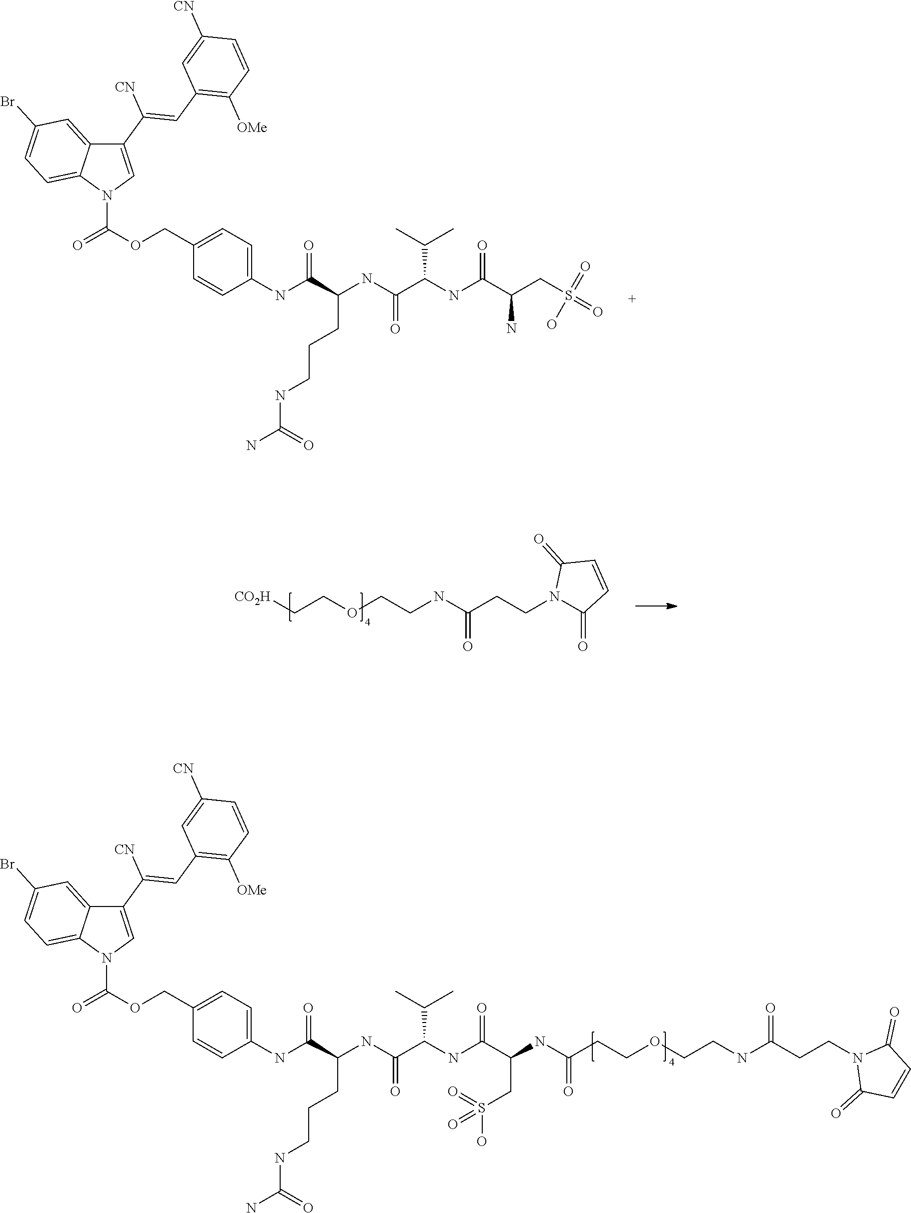

In a 15 ml reactor equipped with magnetic stirring. 19-maleimido-17-oxo-4,7,10,13-tetraoxa-16-azanonadecanoic acid (77 mg. 0.19 mmol) and HBTU (117 mg. 0.31 mmol) were charged in DMF (1.5 mL). Then DIPEA (94 μL, 0.54 mmol) was added. The solution was stirred at RT for 20 min followed by addition of 2-amino-3-[[(1S)-1-[[(1S)-1-[[4-[[5-bromo-3-[(Z)-1-cyano-2-(5-cyano-2-methoxy-phenyl)vinyl]indole-1-carbonyl]oxymethyl]phenyl]carbamoyl]-4-ureido-butyl]carbamoyl]-2-methyl-propyl]amino]-3-oxo-propane-1-sulfonic acid (150 mg, 0.15 mmol). The reaction mixture was stirred at RT for 18 h. The solution was poured in 0.2N HCl, extracted with DCM, the organic layer washed with water, dried over Na₂SO₄ and concentrated under reduced pressure. The crude oil was directly purified by flash chromatography on SiO₂ column, elution with a gradient from DCM 100% to DCM/MeOH 8/2 to give 90 mg of (2R)-3-[[(1S)-1-[(1S)-1-[[4-[[5-bromo-3-[(Z)-1-cyano-2-(5-cyano-2-methoxy-phenyl)vinyl]indole-1-carbonyl]oxymethyl]phenyl]carbamoyl]-4-ureido-butyl]carbamoyl]-2-methyl-propyl]amino]-2-[3-[2-[3-(2,5-dioxopyrrol-1-yl)propanoylamino]ethoxy]propanoylamino]-3-oxo-propane-1-sulfonic acid as a yellow solid (yield=44%)

LCMS-ESI: [M+H]⁺=1334

### III. Solubility

### A. Protocol

For each compound, three solutions were prepared in HCl 0.01N (pH 1.0) at 500 μM and three solutions in phosphate saline buffer (pH 7.4) at 500 μM. The solutions were prepared by dissolving each compound in the adequate solvent in opaque tubes and stirred 24 hours at room temperature. After 24 hours, for each solution, an aliquot was centrifuged 10 minutes at 3 500 rpm in order to separate the insoluble fraction from the soluble fraction. The supernatant was taken and diluted in H₂O (1/20) before injection in LC-MSMS. After HPLC separation, analytes are detected and quantified by mass spectrometry. A standard curve is performed for each test substance. The solubility of each test substance was determined by calculating the concentration of each supernatant according to the standard curve.

### B: Results

Results of solubility are presented in the table 1 below:

TABLE 1

| Example: | Prodrug | Solubility pH 7.4 | | Solubility pH 1.0 | |
|---|---|---|---|---|---|
| Example 38 of WO | Payload | <5 | μM | <5 | μM |
| 2014/086964 | | | | | |
| Example 7 | Phosphonate (n = 2) | 103 | μM | <5 | μM |
| | Mono Na salt | | | | |
| Example 8 | Phosphonate (n = 3) | 71 | μM | <5 | μM |
| | Mono Na salt | | | | |
| Example 13 | Phosphonate (n = 3) | 45 | μM | 19 | μM |
| | Di Na salt | | | | |
| Example 16 | Phosphonate (n = 3) | 35 | μM | <10 | μM |
| | Di lysine salt | | | | |
| Example 6 | Phosphonate (n = 4) | 206 | μM | 34 | μM |
| | Mono Na salt | | | | |
| Example 10 | Phosphonate (n = 6) | 141 | μM | 93 | μM |
| | Mono Na salt | | | | |
| Example 14 | Phosphonate (n = 4) | 180 | μM | — | |
| | Di Na salt | | | | |
| Example 15 | Phosphonate (n = 2) | — | | — | |
| | Di lysine salt | | | | |
| Example 1 | Phosphonate (n = 4) | 497 | μM | — | |
| Example 3 | Phosphonate (n = 6) | 294 | μM | — | |
| Example 2 | Phosphonate (n = 2) | 12 | μM | — | |
| Example 17 | Phosphonate (n = 2) | 510 | μM | — | |
| | carbamate | | | | |
| Example 18 | Phosphonate (n = 4) | 298 | μM | — | |
| | carbamate | | | | |
| Example 21 | Peptidic | 471 | μM | — | |
| | (NH₂-Val-Cit-PABC) | | | | |
| Example 24 | Peptidic | 201 | μM | — | |
| | (Boc-Cysteic acid-Val-Cit- | | | | |
| | PABC) | | | | |
| Example 19 | Glucuronide | 452 | μM | — | |
| Example 22 | Peptidic | 2 | μM | — | |
| | (Mal-PEG4-Val-Cit- | | | | |
| | PABC) | | | | |
| Example 23 | Peptidic | — | | — | |
| | (Mal-PEG24-Val-Cit- | | | | |
| | PABC) | | | | |
| Example 26 | Peptidic | 175 | μM | — | |
| | (Mal-PEG4-Cysteic acid- | | | | |
| | Val-Cit-PABC) | | | | |
| Example 54 of WO | Payload | <1 | μM | <1 | μM |
| 2014/086964 | | | | | |
| Example 4 | Phosphonate (n = 4) | 33 | μM | — | |
| Example 5 | Phosphonate (n = 6) | — | | — | |
| Example 11 | Phosphonate (n = 4) | 476 | μM | 10 | μM |
| | Mono Na salt | | | | |
| Example 12 | Phosphonate (n = 6) | 465 | μM | 109 | μM |
| | Mono Na salt | | | | |

### IV. In-Vitro Evaluation: Cathepsin-B Mediated Drug Release

### Protocole

### Example 27: Synthesis of N-acetyl-S-(1-((R)-2-(((S)-1-(((S)-1-((4-(((5-bromo-3-((Z)-1-cyano-2-(5-cyano-2-methoxyphenyl)vinyl)-1H-indole-1-carbonyl)oxy)methyl)phenyl)amino)-1-oxo-5-ureidopentan-2-yl)amino)-3-methyl-1-oxobutan-2-yl)carbamoyl)-4,20-dioxo-1-sulfo-7,10,13,16-tetraoxa-3,19-diazadocosan-22-yl)-2,5-dioxopyrrolidin-3-yl)-L-cysteine (Quenched mal Prodrug)

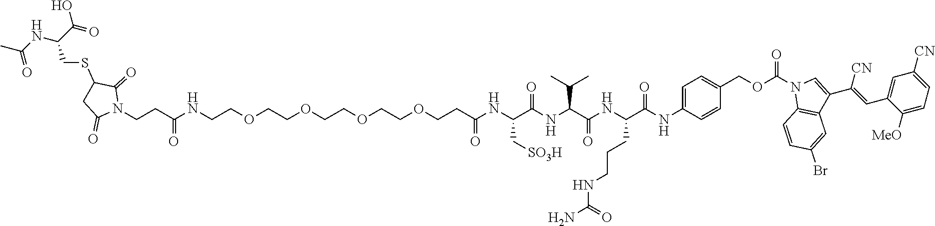 [SMILES: COc1ccc(C#N)cc1/C=C(\C#N)c1cn(C(=O)OCc2ccc(NC(=O)[C@H](CCCNC(N)=O)NC(=O)[C@@H](NC(=O)[C@H](CS(=O)(=O)O)NC(=O)CCOCCOCCOCCOCCNC(=O)CCN3C(=O)CC(SC[C@H](NC(C)=O)C(=O)O)C3=O)C(C)C)cc2)c2ccc(Br)cc12]

A solution of Example 26 (crude material from the previous step), (60 mg. 0.045 mmol) in DMSO 0.8 ml was treated with NAcCys, (8 mg. 0.049 mmol) in 0.2 mL of DMSO; DIPEA (16 uL, 0.09 mmol) added at room temperature to the mixture. Stirred 1 h: UPLC showed completed conversion to the title compound. HCOOH (0.1 mmol, 4 uL) added and pH checked (4, orange to paper Ph 1-12). Crude purified via preparative RP_HPLC. Eluents: Acetonitrile (+0.1% HCOOH;) and water (+0.1% HCOOH;). Column: XBridge PROTEIN, 150×19 mm, 300 A, 5 um. Flow rate: 30 ml/min. Fractions containing the title compound were collected and freeze dried. Recovered 16 mg of a white solid. UPLC-MS (M3): Rt=3.86 peak observed m/z (ES+)=1497.2 [MH]+ consistent with the expected (MW: 1496.42); purity (peak area %) 93%

Drug release of (Z)-3-(2-(5-bromo-1H-indol-3-yl)-2-cyanovinyl)-4-methoxybenzonitrile from Example 27 was performed using cathepsin B enzyme.

Cathepsin B (50 units/mL) was preincubated in activation buffer containing 25 mM sodium acetate (pH 5.5), 30 mM DTT and 15 mM EDTA for 15 min at 37° C. and then added as a 20% (v/v) addition to initiate each reaction.

Final in vitro cathepsin B reaction mixtures will contain 25 mM sodium acetate (pH 5.5), 10 units/mL activated Cathepsin B, 30 mM DTT and 15 mM EDTA, and 0.5 μg/mL of Example 27 in a 2.5% (v/v) incubation sample background.

Incubation will occur for 2 hours at 37° C. (0-5-10-15-30-60-120 min).

Reactions will be terminated by addition of organic solvent and the supernatant analyzed by LC-MS to quantify released free drug.

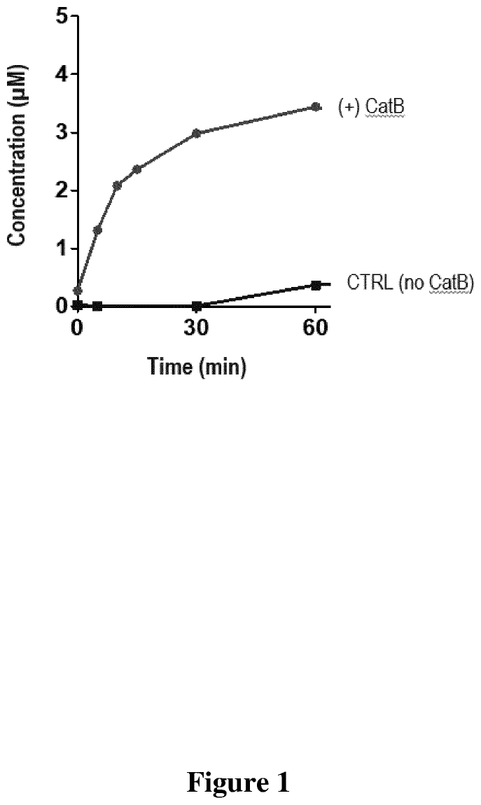

The results of FIG. 1 show an efficient drug release of (Z)-3-(2-(5-bromo-1H-indol-3-yl)-2-cyanovinyl)-4-methoxybenzonitrile.

1-15. (canceled)

16. A compound of formula (I): 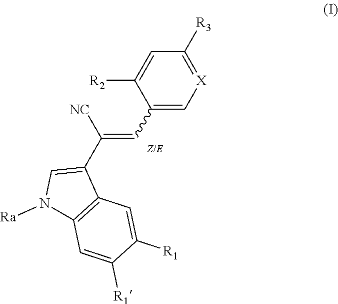
wherein:
X represents a nitrogen atom, a C—CN unit or a N+—O— unit;
R₁ and R₁′ represent independently a hydrogen, a halogen, a (C₁-C₆)alkoxy, or a —SO₂—CH₃ group, with the proviso that if R₁ or R₁′ is a (C₁-C₆)alkoxy, then R² is not a halogen;
R₂ represents:
a radical (C₁-C₆)alkoxy, (C₃-C₆)cycloalkoxy, (C₃-C₆) heterocycloalkoxy, aryloxy, heteroaryloxy, (C₁-C₆)alkyl-aryloxy, (C₁-C₆)alkyl-heteroaryloxy, said radicals being optionally substituted by at least one halogen, or a radical thio-(C₁-C₆)alkyl, thio-aryl, thio-heteroaryl, thio-(C₁-C₆)alkyl-aryl or thio-(C₁-C₆)-alkyl-heteroaryl, said radicals being optionally substituted by at least one halogen or by a (C₁-C₆)alkoxy group,
a —NR₄R₅ unit, a O—(C₁-C₆)alkyl-NR₄R₅ unit or a S—(C₁-C₆)alkyl-NR₄R₅ unit wherein R⁴ and R₅ represent H, a (C₁-C₆)alkyl group, or R₄ and R₅ taken together form a 3- to 7-membered ring, optionally interrupted by one or several heteroatoms, with the proviso that at least one among R₄ and R₅ is not H,
a NHCOR₆ unit wherein R⁶ represents (C₁-C₆)alkyl group,
an aryl or a heteroaryl group optionally substituted by at least one halogen, a trifluoromethyl group, or a (C₁-C₃)alkoxy group, or
a halogen;
R₃ represents a hydrogen, a (C₁-C₃)alkyl group, a (C₁-C₃)alkoxy group or a halogen; and
Ra is a radical selected in a group consisting of:
a group of formula (A): 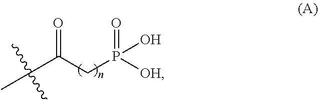
or formula (A′): 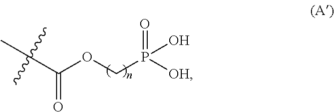
in which n is an integer comprised between 1 and 12; and
a group of formula (B): 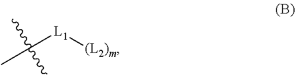
in which:
L₁ is a cleavable group selected from the group consisting of a pH-sensitive group, a photo-induced cleavable group, a bioreductible cleavable group, and an enzymatic cleavable group;
L₂ is a tert-butoxycarbonyl group or a binding protein connector; and
m is an integer equal to 0 or 1;
or a pharmaceutically acceptable salt thereof.

17. The compound of formula (I) according to claim 16, wherein R₂ represents:
a radical (C₁-C₆)alkoxy,
a radical (C₃-C₆) heterocycloalkoxy, or
a heteroaryl group.

18. The compound of formula (I) according to claim 16, wherein:
X represents a C—CN unit,
R₁ represents a halogen,
R₁′ represents a hydrogen,
R₂ represents a radical (C₁-C₆)alkoxy, and
R₃ represents a hydrogen.

19. The compound of formula (I) according to claim 16, wherein:
X represents a C—CN unit,
R₁ represents a hydrogen,
R₁′ represents a radical (C₁-C₆)alkoxy,
R₂ represents a radical (C₁-C₆)alkoxy, and
R₃ represents hydrogen.

20. The compound of formula (I) according to claim 16, wherein Ra is a group of formula (A): 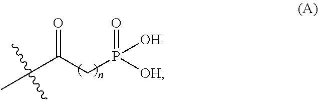
or formula (A′): 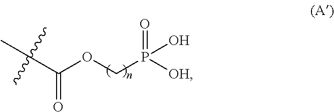
in which n is an integer between 1 and 12.

21. The compound of formula (I) according to claim 16, wherein said pharmaceutically acceptable salt is a sodium salt, a disodium salt, a lysine salt, a dilysine salt, an arginine salt or a diarginine salt.

22. The compound of formula (I) according to claim 16, wherein said compound is selected from the group consisting of:
Example 1: (Z)-5-(5-bromo-3-(1-cyano-2-(5-cyano-2-methoxyphenyl)vinyl)-1H-indol-1-yl)-5-oxopentylphosphonic acid;
Example 2: (Z)-3-(5-bromo-3-(1-cyano-2-(5-cyano-2-methoxyphenyl)vinyl)-1H-indol-1-yl)-3-oxopropylphosphonic acid;
Example 3: (Z)-7-(5-bromo-3-(1-cyano-2-(5-cyano-2-methoxyphenyl)vinyl)-1H-indol-1-yl)-7-oxoheptylphosphonic acid;
Example 4: (Z)-5-(3-(1-cyano-2-(5-cyano-2-methoxyphenyl)vinyl)-6-methoxy-1H-indol-1-yl)-5-oxopentylphosphonic acid;
Example 5: (Z)-7-(3-(1-cyano-2-(5-cyano-2-methoxyphenyl)vinyl)-6-methoxy-1H-indol-1-yl)-7-oxoheptylphosphonic acid;
Example 6: Sodium (Z)-hydrogen5-(5-bromo-3-(1-cyano-2-(5-cyano-2-methoxyphenyl)vinyl)-1H-indol-1-yl)-5-oxopentylphosphonate;
Example 7: sodium (Z)-hydrogen3-(5-bromo-3-(1-cyano-2-(5-cyano-2-methoxyphenyl)vinyl)-1H-indol-1-yl)-3-oxopropylphosphonate;
Example 8: sodium (Z)-hydrogen4-(5-bromo-3-(1-cyano-2-(5-cyano-2-methoxyphenyl)vinyl)-1H-indol-1-yl)-4-oxobutylphosphonate;
Example 9: sodium (Z)-hydrogen6-(5-bromo-3-(1-cyano-2-(5-cyano-2-methoxyphenyl)vinyl)-1H-indol-1-yl)-4-oxohexylphosphonate;
Example 10: sodium (Z)-hydrogen7-(5-bromo-3-(1-cyano-2-(5-cyano-2-methoxyphenyl)vinyl)-1H-indol-1-yl)-7-oxoheptylphosphonate;
Example 11: sodium (Z)-hydrogen5-(3-(1-cyano-2-(5-cyano-2-methoxyphenyl)vinyl)-6-methoxy-1H-indol-1-yl)-5-oxopentylphosphonate;
Example 12: sodium (Z)-hydrogen7-(3-(1-cyano-2-(5-cyano-2-methoxyphenyl)vinyl)-6-methoxy-1H-indol-1-yl)-7-oxoheptylphosphonate;
Example 13: disodium (Z)-4-(5-bromo-3-(1-cyano-2-(5-cyano-2-methoxyphenyl)vinyl)-1H-indol-1-yl)-4-oxobutylphosphonate;
Example 14: disodium (Z)-5-(5-bromo-3-(1-cyano-2-(5-cyano-2-methoxyphenyl)vinyl)-1H-indol-1-yl)-5-oxopentylphosphonate;
Example 15: (S)-2,6-diaminohexanoic acid compound with (Z)-3-(5-bromo-3-(1-cyano-2-(5-cyano-2-methoxyphenyl)vinyl)-1H-indol-1-yl)-3-oxopropylphosphonic acid (2:1);
Example 16: (S)-2,6-diaminohexanoic acid compound with (Z)-4-(5-bromo-3-(1-cyano-2-(5-cyano-2-methoxyphenyl)vinyl)-1H-indol-1-yl)-4-oxobutylphosphonic acid (2:1);
Example 17:3-[hydroxy(dioxo)-lambda6-phosphanyl]propyl 5-bromo-3-[(Z)-1-cyano-2-(2-cyano-5-methoxy-phenyl)vinyl]indole-1-carboxylate; and
Example 18:4-[hydroxy(dioxo)-lambda6-phosphanyl]butyl 5-bromo-3-[(Z)-1-cyano-2-(2-cyano-5-methoxy-phenyl)vinyl]indole-1-carboxylate.

23. The compound of formula (I) according to claim 16, wherein Ra is a group of formula (B): 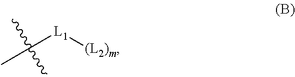
in which:
L₁ is a cleavable group selected from the group consisting of a pH-sensitive group, a photo-induced cleavable group, a bioreductible cleavable group, and an enzymatic cleavable group;
L₂ is a tert-butoxycarbonyl group or a binding protein connector; and
m is an integer equal to 0 or 1.

24. The compound of formula (I) according to claim 23, wherein L₁ is an enzymatic cleavable group cleaved by a protease, a peptidase, an esterase, a beta-glucuronidase, a glycosidase, a phosphodiesterase, a phosphatase, a pyrophosphatase, a tubulin-tyrosine ligase or a lipase.

25. The compound of formula (I) according to claim 23, wherein L₁ is a p-aminobenzyloxycarbonyl-AA1_{w}-AA2ₓ-AA3_{y}-AA4_{z} group with AA1, AA2, AA3, and AA4 represent independently an amino acid selected from the group consisting of alanine, valine, citrulline, phenylalanine, lysine, glycine, aspartic acid, asparagine, glutamic acid, and derivatives thereof, and w, x, y, and z are independently an integer equal to 0 or 1.

26. The compound of formula (I) according to claim 23, wherein m is 1, and L₂ is a binding protein connector having the following formulae: 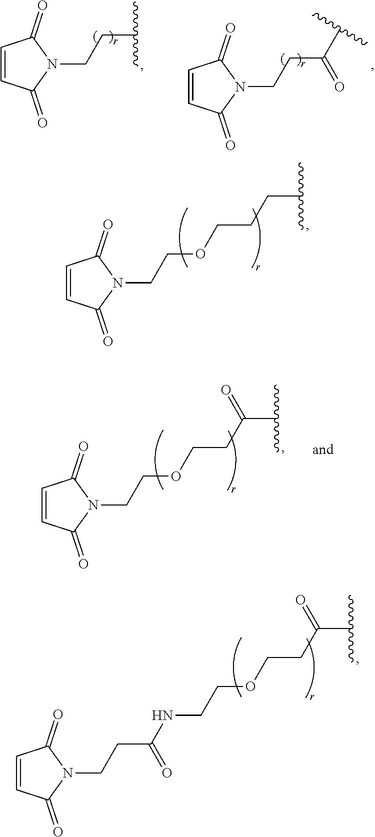
with r is an integer comprised between 1 and 36.

27. The compound of formula (I) according to claim 16, wherein said compound is selected from the group consisting of:
Example 19: (2S,3S,4S,5R,6S)-6-[4-[[5-bromo-3-[(Z)-1-cyano-2-(5-cyano-2-methoxy-phenyl)vinyl]indole-1-carbonyl]oxymethyl]phenoxy]-3,4,5-trihydroxy-tetrahydropyran-2-carboxylic acid;
Example 20: [4-[[(2S)-2-[[(2S)-2-(tert-butoxycarbonylamino)-3-methyl-butanoyl]amino]-5-ureido-pentanoyl]amino]phenyl]methyl-5-bromo-3-[(Z)-1-cyano-2-(5-cyano-2-methoxy-phenyl)vinyl]indole-1-carboxylate;
Example 21: [4-[[(2S)-2-[[(2S)-2-aminopropanoyl]amino]-5-ureido-pentanoyl]amino]phenyl]methyl-5-bromo-3-[(Z)-1-cyano-2-(5-cyano-2-methoxy-phenyl)vinyl]indole-1-carboxylate hydrochloride;
Example 22: [4-[[(2S)-2-[[(2S)-2-[3-[2-[3-(2,5-dioxopyrrol-1-yl)propanoylamino]ethoxy](peg4) propanoylamino]-3-methyl-butanoyl]amino]-5-ureido-pentanoyl]amino]phenyl]methyl-5-bromo-3-[(Z)-1-cyano-2-(5-cyano-2-methoxy-phenyl)vinyl]indole-1-carboxylate;
Example 23: [4-[[(2S)-2-[[(2S)-2-[3-[2-[3-(2,5-dioxopyrrol-1-yl)propanoylamido](peg24) propanoylamino]-3-methyl-butanoyl]amino]-5-ureido-pentanoyl]amino]phenyl]methyl-5-bromo-3-[(Z)-1-cyano-2-(5-cyano-2-methoxy-phenyl)vinyl]indole-1-carboxylate;
Example 24: 3-[[(1S)-1-[(1S)-1-[[4-[[5-bromo-3-[(Z)-1-cyano-2-(5-cyano-2-methoxy-phenyl)vinyl]indole-1-carbonyl]oxymethyl]phenyl]carbamoyl]-4-ureido-butyl]carbamoyl]-2-methyl-propyl]amino]-2-(tert-butoxycarbonylamino)-3-oxo-propane-1-sulfonic acid;
Example 25: 2-amino-3-[(1S)-1-[[(1S)-1-[[4-[[5-bromo-3-[(Z)-1-cyano-2-(5-cyano-2-methoxy-phenyl)vinyl]indole-1-carbonyl]oxymethyl]phenyl]carbamoyl]-4-ureido-butyl]carbamoyl]-2-methyl-propyl]amino]-3-oxo-propane-1-sulfonic acid; and
Example 26: (2R)-3-[[(1S)-1-[[(1S)-1-[[4-[[5-bromo-3-[(Z)-1-cyano-2-(5-cyano-2-methoxy-phenyl)vinyl]indole-1-carbonyl]oxymethyl]phenyl]carbamoyl]-4-ureido-butyl]carbamoyl]-2-methyl-propyl]amino]-2-[3-[2-[3-(2,5-dioxopyrrol-1-yl)propanoylamino]ethoxy]propanoylamino]-3-oxo-propane-1-sulfonic acid.

28. A conjugate of formula (II): 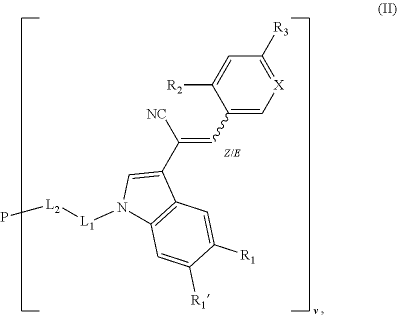
wherein:
X, R₁, R_{1′}, R₂, R³, and L₁ as defined in claim 16;
L₂ is a binding protein connector;
P is a peptide or a protein, an antibody, an antibody fragment, or an antigen-binding fragment, which is able to bind a target of interest; and
v is an integer from 1 to 10.

29. A pharmaceutical composition comprising a compound according to claim 16 and a pharmaceutically acceptable excipient.

30. A method of treating cancer comprising administering a pharmaceutical composition according to claim 29 to a subject in need of treatment.

31. The method according to claim 30, wherein said cancer is leukemia, acute myelogenous leukemia, lymphoma, breast cancer, pancreatic cancer, lung cancer or colon cancer.


SEQUENCE LISTING (4 sequences)

SEQ ID NO: 1 - protein, 4 aa, synthetic construct
  REGION 1..4: note = SEQ03
GGFG

SEQ ID NO: 2 - protein, 4 aa, synthetic construct
  REGION 1..4: note = SEQ04
GFGG

SEQ ID NO: 3 - protein, 4 aa, synthetic construct
  REGION 1..4: note = SEQ05
EGEE

SEQ ID NO: 4 - protein, 4 aa, synthetic construct
  REGION 1..4: note = SEQ06
EEGE